(19) United States
(12) Patent Application Publication
(10) Pub. No.: US 2014/0142180 A1
(43) Pub. Date: May 22, 2014

(54) METHODS OF TREATING TUMORS HAVING ELEVATED MCT1 EXPRESSION

(71) Applicant: Whitehead Institute for Biomedical Research, Cambridge, MA (US)

(72) Inventors: Kivanc Birsoy, Cambridge, MA (US); David M. Sabatini, Cambridge, MA (US); Richard Possemato, Brighton, MA (US); Tim Wang, Boston, MA (US)

(21) Appl. No.: 14/046,643

(22) Filed: Oct. 4, 2013

Related U.S. Application Data
(60) Provisional application No. 61/716,364, filed on Oct. 19, 2012.
(60) Provisional application No. 61/710,414, filed on Oct. 5, 2012.

(51) Int. Cl. A61K 31/19; G01N 33/50

(52) U.S. Cl. CPC A61K 31/19; G01N 33/5011
USPC 514/557; 435/32

(57) ABSTRACT

In some aspects, compositions and methods useful for classifying tumor cells, tumor cell lines, or tumors according to predicted sensitivity to 3-bromopyruvate (3-BrPA) are provided. In some aspects, methods of identifying subjects with cancer who are candidates for treatment with 3-BrPA are provided. In some aspects, compositions useful for subjects with cancers that express increased levels of MCT1 are provided. In some aspects, methods of treating subjects with cancers that express increased levels of MCT1 are provided. In some aspects, methods of identifying anti-tumor agents the efficacy of which is at least in part dependent on transporter-mediated uptake are provided.

### CROSS-REFERENCE TO RELATED APPLICATION

This application claims the benefit of U.S. Provisional Application Nos. 61/710,414, filed Oct. 5, 2012 and 61/716,364, filed Oct. 19, 2012. The entire teachings of the above application(s) are incorporated herein by reference.

### GOVERNMENT FUNDING

This invention was made with government support under R01-CA103866-06 awarded by the National Institutes of Health. The government has certain rights in the invention.

### BACKGROUND

Cancer is a major cause of death worldwide. Cancer cells often acquire metabolic liabilities not shared by their normal counterparts. There is great interest in identifying these liabilities and exploiting them for the development of new cancer therapies. Many cancer cells activate aerobic glycolysis and so exhibit high rates of glucose uptake and lactate excretion even when oxygen is available for oxidative phosphorylation. Efforts to target a number of different glycolytic enzymes for anti-cancer therapy are underway.

### SUMMARY

The entire teachings of U.S. Provisional Application No. 61/710,414 are incorporated herein by reference. In some aspects, the invention relates to methods of predicting sensitivity of tumor cells, tumor cell lines, or tumors to 3-bromopyruvate. In some aspects, the disclosure provides a method of classifying a tumor according to predicted sensitivity to a compound, wherein the compound is 3-bromopyruvate (3-BrPA) or an analog thereof, the method comprising: assessing expression of the MCT1 gene in the tumor or in a sample obtained from the tumor, wherein an increased level of MCT1 expression is correlated with increased sensitivity to the compound; and classifying the tumor with respect to predicted sensitivity to the compound based at least in part on the level of expression of the MCT1 gene in the tumor or sample. In some embodiments the method comprises: (a) determining the level of an MCT1 gene product in the tumor or sample; and (b) comparing the level of MCT1 gene product with a reference level, wherein if the level determined in (a) is greater than the reference level, the tumor is classified as having an increased likelihood of being sensitive to the compound than if the level determined in (a) is not greater than the reference level.

In some aspects, the disclosure provides a method of predicting the likelihood that a tumor cell, tumor cell line, or tumor, is sensitive to a compound, wherein the compound is 3-bromopyruvate (3-BrPA) or an analog thereof, the method comprising: assessing expression of the MCT1 gene by the tumor cell, tumor cell line, or tumor; and generating a prediction of the likelihood that the tumor cell, tumor cell line, or tumor, is sensitive to the compound, wherein if the tumor cell, tumor cell line, or tumor, has increased expression of the MCT1 gene, the tumor cell, tumor cell line, or tumor, is predicted to have increased likelihood of being sensitive to the compound. In some embodiments assessing expression of the MCT1 gene comprises (a) determining the level of an MCT1 gene product in the tumor cell, tumor cell line, tumor, or a sample obtained therefrom; and (b) comparing the level with a reference level of the MCT1 gene product, wherein if the level determined in (a) is greater than the reference level, the tumor cell, tumor cell line, or tumor has increased likelihood of being sensitive to the compound than if the level determined in (a) is not greater than the reference level.

In some aspects, the disclosure provides method of determining whether a subject in need of treatment for a tumor is a candidate for treatment with a compound, wherein the compound is 3-bromopyruvate (3-BrPA) or an analog thereof, the method comprising determining whether the tumor has increased expression of the MCT1 gene; and identifying the subject as a candidate for treatment with the compound if the tumor has increased expression of the MCT1 gene. In some embodiments determining whether the tumor has increased expression of the MCT1 gene comprises (a) determining the level of an MCT1 gene product in the tumor or a sample obtained therefrom; and (b) comparing the level with a reference level of the MCT1 gene product.

In some aspects, the disclosure provides a method of treating a subject in need of treatment for a tumor, the method comprising: (a) determining that the subject's tumor has increased expression of the MCT1 gene; and (b) treating the subject with 3-bromopyruvate (3-BrPA) or an analog thereof. In some embodiments determining that tumor has increased expression of the MCT1 gene comprises (a) determining the level of an MCT1 gene product in the tumor or a sample obtained therefrom; and (b) comparing the level with a reference level of the MCT1 gene product.

In some aspects, the disclosure provides a method of obtaining an assessment of MCT1 expression comprising: providing to a testing facility (a) a sample obtained from a subject in need of treatment for a tumor; and (b) instructions to assess MCT1 expression in the sample. In some embodiments the method further comprises (c) receiving a result of the assessment and; (b) treating or selecting a treatment for a subject based at least in part on the result.

In some embodiments a reference level in a method disclosed herein is a level of the gene product in non-tumor tissue or non-tumor cells. In some embodiments a reference level in a method disclosed herein is a level of the gene product in tumor tissue or tumor cells that are sensitive to 3-BrPA. In some embodiments a reference level in a method disclosed herein is a level of the gene product in tumor tissue or tumor cells that are not sensitive to 3-BrPA. In some embodiments a reference level in a method disclosed herein is a level of the gene product in tumor tissue or tumor cells that are resistant to 3-BrPA.

In some embodiments a MCT1 gene product is a MCT1 mRNA. In some embodiments a MCT1 gene product comprises a MCT1 polypeptide. In some embodiments the level of an MCT1 gene product, e.g., an MCT1 polypeptide, is determined by a method comprising: contacting the tumor, tumor cell, or sample with a detection reagent; and detecting the MCT1 gene product based on detecting the detection reagent. In some embodiments an MCT1 gene product is a MCT1 polypeptide, and the level is determined by a method comprising: contacting the tumor, tumor cell, or sample with an antibody that binds to the MCT1 polypeptide; and detecting the MCT1 polypeptide based on binding of the antibody to the polypeptide. In some embodiments, wherein the MCT1 gene product is a MCT1 polypeptide, the level is determined by performing immunohistochemistry (IHC). In some embodiments, wherein the MCT1 gene product is a MCT1 RNA, the level is determined by a method comprising performing hybridization using a probe that binds to MCT1 RNA or a complement thereof. In some embodiments detecting MCT1 RNA comprises performing reverse transcription and amplification, e.g., by PCR. In some embodiments PCR comprises real-time PCR. In some embodiments detecting MCT1 RNA comprises performing fluorescence in situ hybridization.

In some embodiments of any of the above methods, the method further comprises assessing expression of a second gene in a tumor or in a sample obtained from the tumor, wherein the second gene encodes a gene product that promotes MCT1 expression or function. In some embodiments the second gene product is a basigin (BSG) gene product.

In some embodiments of any of the above methods, the method further comprises treating a subject in need of treatment for the tumor with 3-BrPA or an analog thereof based at least in part on the classification, prediction, or determination. In some embodiments any such methods may further comprise treating the subject with (a) a second anti-tumor therapy; (b) a glycolysis inhibitor; and/or (c) a glycolysis inhibitor.

In some embodiments of any of the above methods, the method further comprises storing the result of the assessment, classification, determination, or prediction in a database, optionally in association with a sample identifier or subject identifier.

In some embodiments of any of the above methods, the method further comprises providing the result of an assessment, classification, determination, or prediction to a health care provider. In some embodiments of any of the above methods, the method further comprises providing the result of an assessment, classification, determination, or prediction to a subject, e.g., a subject in need of treatment for the tumor.

In some aspects, the disclosure provides a method of treating a subject in need of treatment for a tumor the method comprising: treating the subject with 3-bromopyruvate (3-BrPA) or an analog thereof, wherein the tumor has been determined to have increased expression of MCT1. In some embodiments the tumor has been determined to have increased expression of MCT1 by assessing the level of an MCT1 gene product in a sample obtained from the tumor. In some embodiments the tumor has been determined to have increased expression of MCT1 by performing IHC on a sample obtained from the tumor.

In some aspects, the disclosure provides a kit comprising: a detection reagent suitable for detecting an MCT1 gene product. In some embodiments the detection reagent is suitable for detecting an MCT1 gene product in a tumor sample. In some embodiments the detection reagent is suitable for performing a method set forth herein. In some embodiments, the agent has been validated for use in a method set forth above or elsewhere herein. In some embodiments the detection reagent comprises an antibody that binds to MCT1 polypeptide. In some embodiments the detection reagent comprises a probe or primer that hybridizes to mRNA encoding an MCT1 polypeptide or a complement thereof. In some embodiments a kit further comprises (i) instructions for using the kit for tumor classification, prediction, or treatment selection; (ii) a substrate or secondary antibody; and/or (iii) a control substance. In some embodiments a kit comprises a label or package insert indicating that the kit is approved by a government regulatory agency for use in tumor classification, prediction, or treatment selection. In some embodiments a kit comprises a label or package insert indicating that the kit is approved by a government regulatory agency for use as a companion diagnostic for identifying patients who are candidates for treatment with 3-BrPa or a 3-BrPA analog.

In some aspects, the disclosure provides an article comprising: (a) a pharmaceutical composition comprising 3-BrPA or a 3-BrPA analog; and (b) a label or package insert indicating that the pharmaceutical composition is approved by a government regulatory agency for treatment of tumors that have increased expression of MCT1. In some embodiments the label or package insert specifies a test to be used to determine whether a tumor has increased expression of MCT1 and/or to determine whether the tumor is within a category for which the pharmaceutical composition is approved for use.

In some aspects, the disclosure provides a method of classifying a tumor cell, tumor cell line, or tumor according to its level of glycolytic activity, the method comprising: (a) assessing expression of at least one High Glycolytic Activity Associated (HGAA) gene or at least one Low Glycolytic Activity Associated (LGAA) gene in the tumor cell, tumor cell line, tumor or in a sample obtained from the tumor, wherein increased expression of HGAA genes is correlated with increased glycolytic activity, and wherein increased expression of LGAA genes is correlated with decreased glycolytic activity; and (b) classifying the tumor cell, tumor cell line, or tumor according to its level of glycolytic activity based on the result of step (a). In some embodiments expression of at least 2, 3, 4, 5, 10, 15, 20, 25, 30, 35, or 40 HGAA genes are assessed. In some embodiments expression of at least 2, 3, 4, 5, 10, 15, 20, 25, 30, 35, or 40 LGAA genes are assessed. In some embodiments expression of at least 2, 3, 4, 5, 10, 15, 20, 25, 30, 35, or 40 HGAA genes are assessed and at least 2, 3, 4, 5, 10, 15, 20, 25, 30, 35, or 40 LGAA genes are assessed.

In some aspects, the disclosure provides a method of predicting the likelihood that a tumor cell, tumor cell line, or tumor, is sensitive to a compound, wherein the compound is 3-bromopyruvate (3-BrPA) or an analog thereof, the method comprising: classifying the tumor cell, tumor cell line, or tumor according to its level of glycolytic activity using any of the methods of classification according to level of glycolytic activity; and generating a prediction of the likelihood that the tumor cell, tumor cell line, or tumor, is sensitive to the compound, wherein if the tumor cell, tumor cell line, or tumor, has high glycolytic activity, the tumor cell, tumor cell line, or tumor, is predicted to have increased likelihood of being sensitive to the compound.

In some aspects, the disclosure provides a method of determining whether a subject in need of treatment for a tumor is a candidate for treatment with a compound, wherein the compound is 3-bromopyruvate (3-BrPA) or an analog thereof, the method comprising determining whether the tumor has high glycolytic activity using any of the methods of classification according to level of glycolytic activity; and identifying the subject as a candidate for treatment with the compound If the tumor has high glycolytic activity.

In some aspects, the disclosure provides a method of classifying a tumor according to predicted sensitivity to a compound, wherein the compound is 3-bromopyruvate (3-BrPA) or an analog thereof, the method comprising: determining the level of a gene product in a sample obtained from the tumor, wherein the gene product promotes MCT1 expression or function, wherein a decreased level of the gene product is correlated with decreased sensitivity to 3-BrPA, thereby classifying the tumor with respect to predicted sensitivity to the compound. In some embodiments the method comprises (a) determining the level of the gene product in the sample; and (b) comparing the level of the gene product with a reference level, wherein if the level determined in (a) is less than the reference level, the tumor is classified as having an decreased likelihood of being sensitive to the compound. In some embodiments the gene product is a basigin (BSG) gene product.

In some aspects, the disclosure provides a method of modulating sensitivity of a cell to 3-BrPA or an analog thereof, the method comprising modulating the level or activity of MCT1 in the cell. In some embodiments the method comprises increasing the level or activity of MCT1 in the cell, thereby increasing sensitivity of the cell to 3-BrPA or an analog thereof.

In some aspects, the disclosure provides method of identifying a candidate agent for modulating sensitivity of a cell to 3-BrPA or an analog thereof, the method comprising: (a) providing a test agent; and (b) determining whether the test agent modulates expression or activity of a MCT1 gene product, wherein the test agent is identified as a candidate agent for modulating sensitivity of a cell to 3-BrPA or an analog thereof if the test agent modulates expression or activity of a MCT1 gene product. In some embodiments the method comprises determining whether the test agent modulates expression or activity of an MCT1 gene product comprises (i) contacting the test agent with one or more cells that express a MCT1 gene product; and (ii) measuring the level of expression or activity of the MCT1 gene product; wherein an alteration in expression or activity of the MCT1 gene product relative to control cell(s) not exposed to the test agent is indicative that the test agent modulates expression or activity of the MCT1 gene product. In some embodiments a method comprises testing the effect of an identified candidate agent on cells in combination with 3-BrPA. In some embodiments a method comprises preparing a composition comprising an identified candidate agent and a pharmaceutically acceptable carrier. In some embodiments a method comprises preparing a composition comprising an identified candidate agent and 3-BrPA. In some embodiments a method comprises testing the effect of an identified candidate agent on tumor cell survival or proliferation. In some embodiments a method comprises testing the effect of an identified candidate agent on a tumor in vivo, e.g., in a non-human animal that serves as a tumor model. In some embodiments an identified candidate agent is tested in combination with 3-BrPA or an analog thereof.

In some aspects, the disclosure provides a method of modulating sensitivity of a cell to 3-BrPA or an analog thereof, the method comprising modulating the level or activity of GAPDH in the cell. In some embodiments the method comprises decreasing the level or activity of GAPDH in the cell, thereby increasing sensitivity of the cell to 3-BrPA or an analog thereof. In some embodiments the method comprises contacting the cell with a GAPDH inhibitor, thereby increasing sensitivity of the cell to 3-BrPA or an analog thereof.

In some aspects, the disclosure provides a method of identifying a candidate agent for modulating sensitivity of a cell to 3-BrPA or an analog thereof, the method comprising: (a) providing a test agent; (b) determining whether the test agent modulates expression or activity of a GAPDH gene product; and (c) identifying the test agent as a candidate agent for modulating sensitivity of a tumor cell to 3-BrPA or an analog thereof if the test agent modulates expression or activity of a GAPDH gene product. In some embodiments a test agent is identified as a candidate agent for enhancing sensitivity of a tumor cell to 3-BrPA or an analog thereof if the test agent inhibits expression or activity of the GAPDH gene product. In some embodiments determining whether the test agent modulates expression or activity of a GAPDH gene product comprises (i) contacting the test agent with one or more cells that express a GAPDH gene product; and (ii) measuring the level of expression or activity of the GAPDH gene product; wherein an alteration in expression or activity of the GAPDH gene product relative to control cell(s) not exposed to the test agent is indicative that the test agent modulates expression or activity of the GAPDH gene product. In some embodiments the method further comprises testing the effect of an identified candidate agent on cells in combination with 3-BrPA. In some embodiments a method comprises preparing a composition comprising an identified candidate agent and a pharmaceutically acceptable carrier. In some embodiments a method comprises preparing a composition comprising an identified candidate agent and 3-BrPA. In some embodiments a method comprises testing the effect of an identified candidate agent on tumor cell survival or proliferation. In some embodiments a method comprises testing the effect of an identified candidate agent on a tumor in vivo, e.g., in a non-human animal that serves as a tumor model. In some embodiments an identified candidate agent is tested in combination with 3-BrPA or an analog thereof.

In some aspects, the disclosure provides method of testing a candidate agent for modulating sensitivity of a tumor cell to 3-BrPA or an analog thereof, the method comprising: (a) contacting one or more tumor cells with 3-BrPA or an analog thereof and a GAPDH inhibitor; (b) assessing survival or proliferation of the one or more tumor cells. In some embodiments the method further comprises comparing survival or proliferation of the one or more tumor cells with survival or proliferation of one or more tumor cells contacted with the GAPDH inhibitor or with 3-BrPA or an analog thereof as a single agent. In some embodiments the method further comprises identifying the test agent as a modulator of tumor cell sensitivity to 3-BrPA or an analog thereof if the presence of the GAPDH inhibitor reduces survival or proliferation of the one or more tumor cells as compared with survival or proliferation of the one or more tumor cells in the presence of the 3-BrPA or an analog thereof as a single agent.

In some aspects, the disclosure provides method of treating a subject in need of treatment for a tumor having increased expression of MCT1, the method comprising: treating the subject with 3-BrPA or an analog thereof and a GAPDH inhibitor.

In some aspects, the disclosure provides a composition comprising 3-BrPA or an analog thereof and a GAPDH inhibitor.

In some aspects, the disclosure provides a method of testing an anti-tumor therapy, the method comprising (a) providing a subject having a tumor that has increased expression of MCT1; (b) treating the subject with 3-BrPA or an analog thereof and an MCT1 inhibitor; and (c) determining the effect of the 3-BrPA or analog thereof and MCT1 inhibitor on the tumor.

In some aspects, the disclosure provides a method of treating a subject in need of treatment for a tumor having increased expression of MCT1, the method comprising: treating the subject with 3-BrPA or an analog thereof and an MCT1 inhibitor.

In some aspects, the disclosure provides a composition comprising 3-BrPA or an analog thereof and an MCT1 inhibitor. In some embodiments the composition further comprises a pharmaceutically acceptable carrier.

In some aspects, the disclosure provides a method of testing the ability of an agent to inhibit the survival and/or proliferation of a cell comprising (a) contacting one or more test cells with an agent, wherein the one or more test cells has increased expression of a transporter as compared to one or more control cells; (b) assessing the level of inhibition of the survival and/or proliferation of the one or more test cells by the agent; and (c) comparing the level of inhibition of the survival and/or proliferation of the one or more test cells by the agent with the level of inhibition of survival and/or proliferation of control cells by the agent. In some embodiments the transporter is characterized in that it is expressed at increased levels by at least some tumors as compared with non-tumor cells of the same cell type or tissue of origin. In some embodiments the method further comprises (d) identifying the agent as a candidate anti-tumor agent if the level of inhibition of the survival and/or proliferation of the one or more test cells by the agent is greater than the level of inhibition of survival and/or proliferation of control cells by the agent. In some embodiments the method comprises (a) contacting one or more test cells and one or more control cells with the agent; and (b) assessing the level of inhibition of the survival and/or proliferation of the one or more test cells and the one or more control cells by the agent. In some embodiments the one or more test cells express the transporter or mRNA encoding the transporter at a level at least five times as great as the one or more control cells. In some embodiments the one or more test cells and one or more control cells are genetically matched. In some embodiments the one or more test cells and one or more control cells are in a co-culture.

In some aspects, the disclosure provides a method of identifying a candidate anti-tumor agent comprising: (a) contacting one or more test cells with an agent, wherein the one or more test cells has increased expression of a gene that encodes a transporter as compared with expression of the gene by one or more control cells; and (b) assessing the level of inhibition of survival or proliferation of the one or more test cells by the agent. In some embodiments the transporter is characterized in that it is expressed at increased levels by at least some tumors as compared with non-tumor cells of the same cell type or tissue of origin. In some embodiments the method further comprises (c) comparing the level of inhibition of survival or proliferation of the one or more test cells by the agent with the level of inhibition of survival or proliferation of control cells by the agent; and (d) identifying the agent as a candidate anti-tumor agent if the agent has a greater inhibitory effect on survival or proliferation of the one or more test cells than it has on control cells. In some embodiments a method comprises contacting one or more control cells with the agent and assessing the level of inhibition of survival or proliferation of the one or more test cells by the agent. In some embodiments the method further comprises (a) contacting one or more test cells and one or more control cells with the agent. In some embodiments the one or more test cells and one or more control cells are in a co-culture. In some embodiments the one or more test cells, one or more control cells, or both, are tumor cells. In some embodiments the one or more test cells are genetically modified to express the transporter at increased levels or the one or more control cells are genetically modified to express the transporter at decreased levels. In some embodiments the one or more test cells and the one or more control cells are distinguishable based on one or more characteristics other than expression level of the transporter. In some embodiments the transporter is an SLC family member. In some embodiments the transporter is an SLC16 family member. In some embodiments the transporter is MCT1. In some embodiments a method of identifying a candidate anti-tumor agent further comprises administering an agent identified as a candidate anti-tumor agent to an animal that serves as a tumor model and assessing the effect of the agent on tumor formation, development, or growth. In some embodiments a method of identifying a candidate anti-tumor agent further comprises administering an agent identified as a candidate anti-tumor agent to a subject in need of treatment for a tumor that expresses an increased level of the transporter.

In some aspects, the disclosure provides a method of inhibiting survival or proliferation of a tumor cell comprising: (a) determining that the tumor cell expresses an increased level of a transporter; and (b) contacting the tumor cell with a toxic agent, the toxicity of which depends at least in part on uptake by the transporter. In some embodiments the cell is expression of the transporter is a major determinant of sensitivity to the toxic agent. In some embodiments step (a) comprises assessing expression of at least 2 different transporters and identifying at least one transporter that is expressed at an increased level by the cell. In some embodiments the tumor cell is contacted with the toxic agent in culture. In some embodiments the tumor cell is contacted with the toxic agent by administering the toxic agent to a subject having a tumor.

In some aspects, the disclosure provides a method of treating a subject in need of treatment for a tumor: (a) determining that the tumor expresses an increased level of a transporter; and (b) treating the subject with a toxic agent, the toxicity of which depends at least in part on uptake by the transporter. In some embodiments step (a) comprises assessing expression of at least 2 different transporters and identifying at least one transporter that is expressed at an increased level by the cell.

In some embodiments of any aspect described herein relating to 3-BrPA or an analog thereof, the compound is 3-BrPA.

In some embodiments of any aspect described herein, a tumor may be a highly glycolytic tumor.

In some embodiments of any aspect described herein a tumor may be of any tumor type. In some embodiments a tumor may be a carcinoma. In some embodiments a tumor may be a liver tumor, breast tumor, glioblastoma, colon, or cervical tumor. In some embodiments a breast tumor is estrogen receptor (ER) negative.

The practice of certain aspects of the present invention may employ conventional techniques of molecular biology, cell culture, recombinant nucleic acid (e.g., DNA) technology, immunology, transgenic biology, microbiology, nucleic acid and polypeptide synthesis, detection, manipulation, and quantification, and RNA interference that are within the ordinary skill of the art. See, e.g., Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, ^{3rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988. Information regarding diagnosis and treatments of various diseases, including cancer, is found in Longo, D., et al. (eds.), Harrison's Principles of Internal Medicine, 18th Edition; McGraw-Hill Professional, 2011. Information regarding various therapeutic agents and human diseases, including cancer, is found in Brunton, L., et al. (eds.) Goodman and Gilman's The Pharmacological Basis of Therapeutics, 12^{th} Ed., McGraw Hill, 2010 and/or Katzung, B. (ed.) Basic and Clinical Pharmacology, McGraw-Hill/Appleton & Lange; 11th edition (July 2009). All patents, patent applications, books, articles, documents, databases, websites, publications, references, etc., mentioned herein are incorporated by reference in their entirety. In case of a conflict between the specification and any of the incorporated references, the specification (including any amendments thereof), shall control. Applicants reserve the right to amend the specification based, e.g., on any of the incorporated material and/or to correct obvious errors. None of the content of the incorporated material shall limit the invention. Standard art-accepted meanings of terms are used herein unless indicated otherwise. Standard abbreviations for various terms are used herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1. Haploid cell genetic screening identifies MCT1 as required for 3-BrPA sensitivity. (a) Mutagenized KBM7 cells were treated with 3-BrPA and resistant clones were pooled. Gene-trap insertion sites were identified by massively parallel sequencing and mapped to the human genome. The Y-axis represents the proximity index, a measure of the local density of insertions. The X-axis represents the insertion sites ordered by their genomic position. (b) Map of unique insertion sites in the SLC16A1 (MCT1) and BSG (Basigin) genes in the surviving cell population. Boxes denote exons. (c) Immunoblotting for MCT1 protein in two clonally derived cell lines containing gene trap insertions in SLC 16A1 (Clone A and B). (d) Resistance of MCT1-null KBM7 clones to 3-BrPA (50 μM) compared to wild type KBM7 cells. Microscopic analysis (Left) and survival curves (Right) of wild type and MCT1 null KBM7 cells after 3 days of 3-BrPA treatment. (e) Exogenous expression of MCT1 to MCT1-null KBM7 cells restores their sensitivity to 3-BrPA. Error bars are ±SEM (n=3).

FIG. 2. MCT1-null cells are immune to the metabolic effects of 3-BrPA and do not take it up. (a) Extracellular Flux Analysis of wild type and MCT1-null KBM7 cells upon 3-BrPA (50 μM) addition. Changes in ECAR, a proxy for lactate production, were monitored upon the addition of 50 μM 3-BrPA. Results are displayed as a percentage of the ECAR reading immediately before 3-BrPA addition. Error bars are +SEM (n=10). (b) Intracellular ATP levels in wild type and MCT1-null KBM7 cells were determined after treatment for 30 minutes with the indicated concentrations of 3-BrPA using a luciferase-based assay. Error bars are ±SEM (n=6). Immunoblots show phosphorylation status of AMPK and ACC in wild type and MCT1-null KBM7 cell after treatment with 3-BrPA (50 μM). (c) Heat map of relative metabolite changes between wild type and MCT1-null KBM7 cells upon 3-BrPA treatment. Yellow to blue colored bar indicates degree of change (log 2) in metabolite abundance relative to MCT1-null KBM7 cells. Cells were cultured for 1 hour with 50 μm 3-BrPA and intracellular metabolites were obtained and analyzed by LC-MS (n=3). (d) ¹⁴C-3-BrPA uptake in MCT1-null and wild type KBM7 cells in the presence/absence of excess unlabeled 3-BrPA (500 μM). Error bars are ±SEM (n=3).

FIG. 3. MCT1 expression is the predominant determinant of 3-BrPA sensitivity in cancer cells. (a) The concentration of 3-BrPA at which 50% cell growth inhibition occurred after 3 days of administration (IC₅₀) was determined for 15 cancer cell lines. These values were correlated with transcriptome-wide mRNA expression data from the Cancer Cell Line Encyclopedia (CCLE) and the resulting Pearson correlation coefficients were sorted and plotted. (b) Immunoblot shows MCT1 protein levels for 9 different breast cancer cell lines (Left). Relative protein levels correlated with the corresponding IC₅₀ values for 3-BrPA of each cell line (Right). (c) Immunoblot for MCT1 levels in SK-BR-3 and MDA-MB-231 cells expressing a control GFP protein or MCT1 (Left). Survival curves of indicated cell lines expressing the MCT1 cDNA and treated with 3-BrPA (Right). Error bars are ±SEM (n=3). (d) ¹⁴C-3-BrPA uptake in parental and MCT1-overexpressing MDA-MB-231 cells. Error bars are ±SEM (n=3). (e) Immunoblots and survival curves upon 3-BrPA treatment for indicated cell lines expressing shRNAs targeting a control GFP protein or MCT1 (MCT1_{—}1 and MCT1_{—}2). Error bars are ±SEM (n=3). (f) Representative photographs (Left) and average weights (Right) of tumors formed by MDA-MB-231 cells expressing the MCT1 or GFP cDNA after 3 weeks of treatment with vehicle or 3-BrPA (8 mg/kg). Error bars are ±SD (n=5).

FIG. 4. MCT1 expression correlates with glycolysis upregulation in cancer cells. (a) OCR/ECAR values were determined for 15 cell lines using the Seahorse Extracellular Flux Analyzer. These values were correlated with transcriptome-wide mRNA expression data from the Cancer Cell Line Encyclopedia (CCLE) and the resulting Pearson correlation coefficients were sorted and plotted. (b) Schematic illustration of the toxic cargo delivery strategy using 3-BrPA. Glycolytic cancer cells express high levels of MCT1 and are sensitive to 3-BrPA. Cancer cells with low/no levels of MCT1 are resistant to 3-BrPA.

FIG. 5. Plot showing OCR/ECAR ratios for 15 tumor cell lines.

FIG. 6. Synthetic lethal effects of combined 3-BrPA and GAPDH inhibition (A) Western blots showing knockdown of GAPDH expression in tumor cell lines BT-549 (left) and MDA-MB-468 (right) by three different short hairpin RNAs targeted to GAPDH. (B) Plots showing percent survival in the presence of different concentrations of 3-BrPA of cells of tumor cell lines BT-549 (left) and MDA-MB-468 (right) expressing either a control shRNA (targeted to GFP) or shRNA targeted to GAPDH.

FIG. S1. A. MCT1 and BSG genes contain the highest degree of insertional enrichment in 3-BrPA selected cells compared to the unselected control cells (p=4.7E-121 and p=5E-29, respectively). Y axis represents the inverse logarithm of p values, calculated by Fisher Exact Test. The X-axis represents the insertion sites ordered by their genomic position. The diameter of the bubbles denotes the number of insertions for each gene. B. Wild type and MCT1 null KBM7 cells were treated for 3 hours with 3-BrPA (50 uM) and FACS analysis were performed using 7AAD and Annexin V staining.

FIG. S2. A. Lactate production of wild type and MCT1 null KBM7 cells were measured using a colorimetric assay and normalized by cell number. ECAR and OCR reading were measured using Seahorse Extracellular Flux Analyzer. AUC (area under curve) converts OCR rate data to accumulation of total oxygen consumed upon three separate readings. B. Extracellular Flux Analysis of wild type and MCT1 null KBM7 cells upon 3-BrPA (50 uM) addition. Changes in OCR, a proxy for oxygen consumption, were monitored upon the addition of 50 uM 3-BrPA. Results are displayed as a percentage of the OCR reading immediately before 3-BrPA addition.

FIG. S3, A. pH dependence of MCT1 mediated 3-BrPA transport. Wild Type KBM7 cells were incubated with 50 uM of radiactively labeled 3-BrPA for 20 minutes in HBSS in presence of indicated pH conditions and various monocarboxylates. (n=3) B. Dose dependent inhibition of labeled 3-BrPA transport by D-Lactate, L-Lactate and Pyruvate. Wild Type KBM7 cells were incubated with 50 uM radiactively labeled 3-BrPA for 20 minutes in presence of different concentrations of indicated monocarboxylates.

FIG. S4. A. IHC staining of MDA-MB-231 tumors expressing GFP and MCT1 cDNAs using an anti-MCT1 antibody. B. Relative expression of MCT1 in cancer cell lines and their normal tissue of origins. Data was collected from CCLE and GEO browser and quantile normalized. C. Top 40 genes correlating with low (glycolytic) and high (OXPHOS) OCR/ECAR values.

### DETAILED DESCRIPTION OF CERTAIN EMBODIMENTS

### I. Glossary

Descriptions and certain information relating to various terms used in the present disclosure are collected here for convenience.

“Agent” is used herein to refer to any substance, compound (e.g., molecule), supramolecular complex, material, or combination or mixture thereof. A compound may be any agent that can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

An “analog” of a first agent refers to a second agent that is structurally and/or functionally similar to the first agent. A “structural analog” of a first agent is an analog that is structurally similar to the first agent. A structural analog of an agent may have substantially similar physical, chemical, biological, and/or pharmacological propert(ies) as the agent or may differ in at least one physical, chemical, biological, or pharmacological property. In some embodiments at least one such property may be altered in a manner that renders the analog more suitable for a purpose of interest. In some embodiments a structural analog of an agent differs from the agent in that at least one atom, functional group, or substructure of the agent is replaced by a different atom, functional group, or substructure in the analog. In some embodiments, a structural analog of an agent differs from the agent in that at least one hydrogen or substituent present in the agent is replaced by a different moiety (e.g., a different substituent) in the analog. In some embodiments an analog may comprise a moiety that reacts with a target to form a covalent bond.

The terms “assessing”, “determining”, “evaluating”, “assaying” are used interchangeably herein to refer to any form of detection or measurement, and include determining whether a substance, signal, disease, condition, etc., is present or not. The result of an assessment may be expressed in qualitative and/or quantitative terms. Assessing may be relative or absolute. “Assessing the presence of” includes determining the amount of something that is present or determining whether it is present or absent.

“Cellular marker” refers to a molecule (e.g., a protein, RNA, DNA, lipid, carbohydrate), complex, or portion thereof, the presence, absence, or level of which in or on a cell (e.g., at least partly exposed at the cell surface) characterizes, indicates, or identifies one or more cell type(s), cell lineage(s), or tissue type(s) or characterizes, indicates, or identifies a particular state (e.g., a diseased or physiological state such as apoptotic or non-apoptotic, a differentiation state, a stem cell state). In some embodiments a cellular marker comprises the presence, absence, or level of a particular modification of a molecule or complex, e.g., a co- or post-translational modification of a protein. A level may be reported in a variety of different ways, e.g., high/low; +/−; numerically, etc. The presence, absence, or level of certain cellular marker(s) may indicate a particular physiological or diseased state of a patient, organ, tissue, or cell. It will be understood that multiple cellular markers may be assessed to, e.g., identify or isolate a cell type of interest, diagnose a disease, etc. In some embodiments between 2 and 10 cellular markers may be assessed. A cellular marker present on or at the surface of cells may be referred to as a “cell surface marker” (CSM). It will be understood that a CSM may be only partially exposed at the cell surface. In some embodiments a CSM or portion thereof is accessible to a specific binding agent present in the environment in which such cell is located, so that the binding agent may be used to, e.g., identify, label, isolate, or target the cell. In some embodiments a CSM is a protein at least part of which is located outside the plasma membrane of a cell. Examples of CSMs include CD molecules, receptors with an extracellular domain, channels, and cell adhesion molecules. In some embodiments, a receptor is a growth factor receptor, hormone receptor, integrin receptor, folate receptor, or transferrin receptor. A cellular marker may be cell type specific. A cell type specific marker is generally expressed or present at a higher level in or on (at the surface of) a particular cell type or cell types than in or on many or most other cell types (e.g., other cell types in the body or in an artificial environment). In some cases a cell type specific marker is present at detectable levels only in or on a particular cell type of interest and not on other cell types. However, useful cell type specific markers may not be and often are not absolutely specific for the cell type of interest. A cellular marker, e.g., a cell type specific marker, may be present at levels at least 1.5-fold, at least 2-fold or at least 3-fold greater in or on the surface of a particular cell type than in a reference population of cells which may consist, for example, of a mixture containing cells from multiple (e.g., 5-10; 10-20, or more) of different tissues or organs in approximately equal amounts. In some embodiments a cellular marker, e.g., a cell type specific marker, may be present at levels at least 4-5 fold, between 5-10 fold, between 10-fold and 20-fold, between 20-fold and 50-fold, between 50-fold and 100-fold, or more than 100-fold greater than its average expression in a reference population. It will be understood that a cellular marker, e.g., a CSM, may be present in a cell fraction, organelle, cell fragment, or other material originating from a cell in which it is present and may be used to identify, detect, or isolate such material. In general, the level of a cellular marker may be determined using standard techniques such as Northern blotting, in situ hybridization, RT-PCR, sequencing, immunological methods such as immunoblotting, immunohistochemistry, fluorescence detection following staining with fluorescently labeled antibodies (e.g., flow cytometry, fluorescence microscopy), similar methods using non-antibody ligands that specifically bind to the marker, oligonucleotide or cDNA microarray, protein microarray analysis, mass spectrometry, etc. A CSM, e.g., a cell type specific CSM, may be used to detect or isolate cells or as a target in order to deliver an agent to cells. For example, the agent may be linked to a moiety that binds to a CSM. Suitable binding moieties include, e.g., antibodies or ligands, e.g., small molecules, aptamers, or polypeptides. Methods known in the art can be used to separate cells that express a cellular marker, e.g., a CSM, from cells that do not, if desired. In some embodiments a specific binding agent can be used to physically separate cells that express a CSM from cells that do not. In some embodiments, flow cytometry is used to quantify cells that express a cellular marker, e.g., a CSM, or to separate cells that express a cellular marker, e.g., a CSM, from cells that do not. For example, in some embodiments cells are contacted with a fluorescently labeled antibody that binds to the CSM. Fluorescence activated cell sorting (FACS) is then used to separate cells based on fluorescence. analyze

“Computer-assisted” as used herein encompasses methods in which a computer is used to gather, process, manipulate, display, visualize, receive, transmit, store, or in any way handle or analyze information (e.g., data, results, structures, sequences, etc.). A method may comprise causing the processor of a computer to execute instructions to gather, process, manipulate, display, receive, transmit, or store data or other information. The instructions may be embodied in a computer program product comprising a computer-readable medium. A computer-readable medium may be any tangible medium (e.g., a non-transitory storage medium) having computer usable program instructions embodied in the medium. Any combination of one or more computer usable or computer readable medium(s) may be utilized in various embodiments. A computer-usable or computer-readable medium may be or may be part of, for example but not limited to, an electronic, magnetic, optical, electromagnetic, infrared, or semiconductor system, apparatus, or device. Examples of a computer-readable medium include, e.g., a hard disk, a random access memory (RAM), a read-only memory (ROM), an erasable programmable read-only memory (e.g., EPROM or Flash memory), a portable compact disc read-only memory (CDROM), a floppy disk, an optical storage device, or a magnetic storage device. In some embodiments a method comprises transmitting or receiving data or other information over a communication network. The data or information may be generated at or stored on a first computer-readable medium at a first location, transmitted over the communication network, and received at a second location, where it may be stored on a second computer-readable medium. A communication network may, for example, comprise one or more intranets or the Internet.

“Detection reagent” refers to an agent that is useful to specifically detect a gene product or other analyte of interest, e.g., an agent that specifically binds to the gene product or other analyte. Examples of agents useful as detection reagents include, e.g., nucleic acid probes or primers that hybridize to RNA or DNA to be detected, antibodies, aptamers, or small molecule ligands that bind to polypeptides to be detected, and the like. In some embodiments a detection reagent comprises a label. In some embodiments a detection reagent is attached to a support. Such attachment may be covalent or noncovalent in various embodiments. Methods suitable for attaching detection reagents or analytes to supports will be apparent to those of ordinary skill in the art. A support may be a substantially planar or flat support or may be a particulate support, e.g., an approximately spherical support such as a microparticle (also referred to as a “bead”, “microsphere”), nanoparticle (or like terms), or population of microparticles. In some embodiments a support is a slide, chip, or filter. In some embodiments a support is at least a portion of an inner surface of a well or other vessel, channel, flow cell, or the like. A support may be rigid, flexible, solid, or semi-solid (e.g., gel). A support may be comprised of a variety of materials such as, for example, glass, quartz, plastic, metal, silicon, agarose, nylon, or paper. A support may be at least in part coated, e.g., with a polymer or substance comprising a reactive functional group suitable for attaching a detection reagent or analyte thereto.

An “effective amount” or “effective dose” of an agent (or composition containing such agent) refers to the amount sufficient to achieve a desired biological and/or pharmacological effect, e.g., when delivered to a cell or organism according to a selected administration form, route, and/or schedule. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered to a subject in a single dose, or through use of multiple doses, in various embodiments

The term “expression” encompasses the processes by which nucleic acids (e.g., DNA) are transcribed to produce RNA, and (where applicable) RNA transcripts are processed and translated into polypeptides.

The term “gene product” (also referred to herein as “gene expression product” or “expression product”) encompasses products resulting from expression of a gene, such as RNA transcribed from a gene and polypeptides arising from translation of such RNA. It will be appreciated that certain gene products may undergo processing or modification, e.g., in a cell. For example, RNA transcripts may be spliced, polyadenylated, etc., prior to mRNA translation, and/or polypeptides may undergo co-translational or post-translational processing such as removal of secretion signal sequences, removal of organelle targeting sequences, or modifications such as phosphorylation, fatty acylation, etc. The term “gene product” encompasses such processed or modified forms. Genomic, mRNA, polypeptide sequences from a variety of species, including human, are known in the art and are available in publicly accessible databases such as those available at the National Center for Biotechnology Information (www.ncbi.nih.gov) or Universal Protein Resource (www.uniprot.org). Databases include, e.g., GenBank, RefSeq, Gene, UniProtKB/SwissProt, UniProtKB/Trembl, and the like. In general, sequences, e.g., mRNA and polypeptide sequences, in the NCBI Reference Sequence database may be used as gene product sequences for a gene of interest. It will be appreciated that multiple alleles of a gene may exist among individuals of the same species. For example, differences in one or more nucleotides (e.g., up to about 1%, 2%, 3-5% of the nucleotides) of the nucleic acids encoding a particular protein may exist among individuals of a given species. Due to the degeneracy of the genetic code, such variations often do not alter the encoded amino acid sequence, although DNA polymorphisms that lead to changes in the sequence of the encoded proteins can exist. Examples of polymorphic variants can be found in, e.g., the Single Nucleotide Polymorphism Database (dbSNP), available at the NCBI website at www.ncbi.nlm.nih.gov/projects/SNP/. (Sherry S T, et al. (2001). “dbSNP: the NCBI database of genetic variation”. Nucleic Acids Res. 29 (1): 308-311; Kitts A, and Sherry S, (2009). The single nucleotide polymorphism database (dbSNP) of nucleotide sequence variation in The NCBI Handbook [Internet]. McEntyre J, Ostell J, editors. Bethesda (MD): National Center for Biotechnology Information (US); 2002 (www.ncbi.nlm.nih.gov/bookshelf/br.fcgi?book=handbook&part=ch5). Multiple isoforms of certain proteins may exist, e.g., as a result of alternative RNA splicing or editing. In general, where aspects of this disclosure pertain to a gene or gene product, embodiments pertaining to allelic variants or isoforms are encompassed, if applicable, unless indicated otherwise. Certain embodiments may be directed to particular sequence(s), e.g., particular allele(s) or isoform(s).

“Identity” or “percent identity” is a measure of the extent to which the sequence of two or more nucleic acids or polypeptides is the same. The percent identity between a sequence of interest A and a second sequence B may be computed by aligning the sequences, allowing the introduction of gaps to maximize identity, determining the number of residues (nucleotides or amino acids) that are opposite an identical residue, dividing by the minimum of TG_{A} and TG_{B} (here TG_{A} and TG_{B} are the sum of the number of residues and internal gap positions in sequences A and B in the alignment), and multiplying by 100. When computing the number of identical residues needed to achieve a particular percent identity, fractions are to be rounded to the nearest whole number. Sequences can be aligned with the use of a variety of computer programs known in the art. For example, computer programs such as BLAST2, BLASTN, BLASTP, Gapped BLAST, etc., may be used to generate alignments and/or to obtain a percent identity. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). In some embodiments, to obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. See the Web site having URL wwwmcbi.nlm.nih.gov and/or McGinnis, S, and Madden, T L, W20-W25 Nucleic Acids Research, 2004, Vol. 32, Web server issue. Other suitable programs include CLUSTALW (Thompson J D, Higgins D G, Gibson T J, Nuc Ac Res, 22:4673-4680, 1994) and GAP (GCG Version 9.1; which implements the Needleman & Wunsch, 1970 algorithm (Needleman S B, Wunsch C D, J Mol Biol, 48:443-453, 1970.) Percent identity may be evaluated over a window of evaluation. In some embodiments a window of evaluation may have a length of at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, e.g., 100%, of the length of the shortest of the sequences being compared. In some embodiments a window of evaluation is at least 100; 200; 300; 400; 500; 600; 700; 800; 900; 1,000; 1,200; 1,500; 2,000; 2,500; 3,000; 3,500; 4,000; 4,500; or 5,000 amino acids. In some embodiments no more than 20%, 10%, 5%, or 1% of positions in either sequence or in both sequences over a window of evaluation are occupied by a gap. In some embodiments no more than 20%, 10%, 5%, or 1% of positions in either sequence or in both sequences are occupied by a gap.

“Inhibit” may be used interchangeably with terms such as “suppress”, “decrease”, “reduce” and like terms, as appropriate in the context. It will be understood that the extent of inhibition may vary. For example, inhibition may refer to a reduction of the relevant level by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%. In some embodiments inhibition refers to a decrease of 100%, e.g., to background levels or undetectable levels. In some embodiments inhibition is statistically significant.

“Isolated” means 1) separated from at least some of the components with which it is usually associated in nature; 2) prepared or purified by a process that involves the hand of man; and/or 3) not occurring in nature, e.g., present in an artificial environment. In some embodiments an isolated cell is a cell that has been removed from a subject, separated from at least some other cells in a cell population, or a cell that remains after at least some other cells in a cell population have been removed or eliminated.

The term “label” (also referred to as “detectable label”) refers to any moiety that facilitates detection and, optionally, quantification, of an entity that comprises it or to which it is attached. In general, a label may be detectable by, e.g., spectroscopic, photochemical, biochemical, immunochemical, electrical, optical, chemical or other means. In some embodiments a detectable label produces an optically detectable signal (e.g., emission and/or absorption of light), which can be detected e.g., visually or using suitable instrumentation such as a light microscope, a spectrophotometer, a fluorescence microscope, a fluorescent sample reader, a fluorescence activated cell sorter, a camera, or any device containing a photodetector. Labels that may be used in various embodiments include, e.g., organic materials (including organic small molecule fluorophores (sometimes termed “dyes”), quenchers (e.g., dark quenchers), polymers, fluorescent proteins); enzymes; inorganic materials such as metal chelates, metal particles, colloidal metal, metal and semiconductor nanocrystals (e.g., quantum dots); compounds that exhibit luminescensce upon enzyme-catalyzed oxidation such as naturally occurring or synthetic luciferins (e.g., firefly luciferin or coelenterazine and structurally related compounds); haptens (e.g., biotin, dinitrophenyl, digoxigenin); radioactive atoms (e.g., radioisotopes such as ³H, ¹⁴C, ³²P, ³⁵S, ¹²⁵I), stable isotopes (e.g., ¹³C, ²H); magnetic or paramagnetic molecules or particles, etc. Fluorescent dyes include, e.g., acridine dyes; BODIPY, coumarins, cyanine dyes, napthalenes (e.g., dansyl chloride, dansyl amide), xanthene dyes (e.g., fluorescein, rhodamines), and derivatives of any of the foregoing. Examples of fluorescent dyes include Cy3, Cy3.5, Cy5, Cy5.5, Cy7, Alexa® Fluor dyes, DyLight® Fluor dyes, FITC, TAMRA, Oregon Green dyes, Texas Red, to name but a few. Fluorescent proteins include green fluorescent protein (GFP), blue, sapphire, yellow, red, orange, and cyan fluorescent proteins and fluorescent variants such as enhanced GFP (eGFP), mFruits such as mCherry, mTomato, mStrawberry; R-Phycoerythrin, etc. Enzymes useful as labels include, e.g., enzymes that act on a substrate to produce a colored, fluorescent, or luminescent substance. Examples include luciferases, beta-galactosidase, horseradish peroxidase, and alkaline phosphatase. Luciferases include those from various insects (e.g., fireflies, beetles) and marine organisms (e.g., cnidaria such as Renilla (e.g., Renilla reniformis, copepods such as Gaussia (e.g., Gaussia princeps) or Metridia (e.g., Metridia longa, Metridia pacifica), and modified versions of the naturally occurring proteins. A wide variety of systems for labeling and/or detecting labels or labeled entities are known in the art. Numerous detectable labels and methods for their use, detection, modification, and/or incorporation into or conjugation (e.g., covalent or noncovalent attachment) to biomolecules such as nucleic acids or proteins, etc., are described in Iain Johnson, I., and Spence, M. T. Z. (Eds.), The Molecular Probes® Handbook—A Guide to Fluorescent Probes and Labeling Technologies. 11th edition (Life Technologies/Invitrogen Corp.) available online on the Life Technologies website at http://www.invitrogen.com/site/us/en/home/References/Molecular-Probes-The-Handbook.html and Hermanson, G T., Bioconjugate Techniques, 2^{nd} ed., Academic Press (2008). Many labels are available as derivatives that are attached to or incorporate a reactive functional group so that the label can be conveniently conjugated to a biomolecule or other entity of interest that comprises an appropriate second functional group (which second functional group may either occur naturally in the biomolecule or may be introduced during or after synthesis). For example, an active ester (e.g., a succinimidyl ester), carboxylate, isothiocyanate, or hydrazine group can be reacted with an amino group; a carbodiimide can be reacted with a carboxyl group; a maleimide, iodoacetamide, or alkyl bromide (e.g., methyl bromide) can be reacted with a thiol (sulfhydryl); an alkyne can be reacted with an azide (via a click chemistry reaction such as a copper-catalyzed or copper-free azide-alkyne cycloaddition). Thus, for example, an N-hydroxysuccinide (NHS)-functionalized derivative of a fluorophore or hapten (such as biotin) can be reacted with a primary amine such as that present in a lysine side chain in a protein or in an aminoallyl-modified nucleotide incorporated into a nucleic acid during synthesis. A label may be directly attached to an entity or may be attached to an entity via a spacer or linking group, e.g., an alkyl, alkylene, aminoallyl, aminoalkynyl, or oligoethylene glycol spacer or linking group, which may have a length of, e.g., between 1 and 4, 4-8, 8-12, 12-20 atoms, or more in various embodiments. A label or labeled entity may be directly detectable or indirectly detectable in various embodiments. A label or labeling moiety may be directly detectable (i.e., it does not require any further reaction or reagent to be detectable, e.g., a fluorophore is directly detectable) or it may be indirectly detectable (e.g., it is rendered detectable through reaction or binding with another entity that is detectable, e.g., a hapten is detectable by immunostaining after reaction with an appropriate antibody comprising a reporter such as a fluorophore or enzyme; an enzyme acts on a substrate to generate a directly detectable signal). A label may be used for a variety of purposes in addition to or instead of detecting a label or labeled entity. For example, a label can be used to isolate or purify a substance comprising the label or having the label attached thereto. The term “labeled” is used herein to indicate that an entity (e.g., a molecule, probe, cell, tissue, etc.) comprises or is physically associated with (e.g., via a covalent bond or noncovalent association) a label, such that the entity can be detected. In some embodiments a detectable label is selected such that it generates a signal that can be measured and whose intensity is related to (e.g., proportional to) the amount of the label. In some embodiments two or more different labels or labeled entities are used or present in a composition. In some embodiments the labels may be selected to be distinguishable from each other. For example, they may absorb or emit light of different wavelengths. In some embodiments the labels may be selected to interact with each other. For example, a first label may be a donor molecule that transfers energy to a second label, which serves as an acceptor molecule through nonradiative dipole-dipole coupling as in resonance energy transfer (RET), e.g., Förster resonance energy transfer (FRET, also commonly nfluorescence resonance energy transfer),

“Modulate” as used herein means to decrease (e.g., inhibit, reduce) or increase (e.g., stimulate, activate) a level, response, property, activity, pathway, or process. A “modulator” is an agent capable of modulating a level, response, property, activity, pathway, or process. A modulator may be an inhibitor, antagonist, activator, or agonist.

“Nucleic acid” is used interchangeably with “polynucleotide” and encompasses polymers of nucleotides. “Oligonucleotide” refers to a relatively short nucleic acid, e.g., typically between about 4 and about 100 nucleotides (nt) long, e.g., between 8-60 nt or between 10-40 nt long. Nucleotides include, e.g., ribonucleotides or deoxyribonucleotides. In some embodiments a nucleic acid comprises or consists of DNA or RNA. In some embodiments a nucleic acid comprises or includes only standard nucleobases (often referred to as “bases”). The standard bases are cytosine, guanine, adenine (which are found in DNA and RNA), thymine (which is found in DNA) and uracil (which is found in RNA), abbreviated as C, G, A, T, and U, respectively. In some embodiments a nucleic acid may comprise one or more non-standard nucleobases, which may be naturally occurring or non-naturally occurring (i.e., artificial; not found in nature) in various embodiments. In some embodiments a nucleic acid may comprise one or more chemically or biologically modified bases (e.g., alkylated (e.g., methylated) bases), modified sugars (e.g., 2′-O-alkyribose (e.g., 2′-O methylribose), 2′-fluororibose, arabinose, or hexose), modified phosphate groups or modified internucleoside linkages (i.e., a linkage other than a phosphodiester linkage between consecutive nucleosides, e.g., between the 3′ carbon atom of one sugar molecule and the 5′ carbon atom of another), such as phosphorothioates, 5′-N-phosphoramidites, alkylphosphonates, phosphorodithioates, phosphate esters, alkylphosphonothioates, phosphoramidates, carbamates, carbonates, phosphate triesters, acetamidates, carboxymethyl esters and peptide bonds). In some embodiments a modified base has a label (e.g., a small organic molecule such as a fluorophore dye) covalently attached thereto. In some embodiments the label or a functional group to which a label can be attached is incorporated or attached at a position that is not involved in Watson-Crick base pairing such that a modification at that position will not significantly interfere with hybridization. For example the C-5 position of UTP and dUTP is not involved in Watson-Crick base-pairing and is a useful site for modification or attachment of a label. In some embodiments a “modified nucleic acid” is a nucleic acid characterized in that (1) at least two of its nucleosides are covalently linked via a non-standard internucleoside linkage (i.e., a linkage other than a phosphodiester linkage between the 5′ end of one nucleotide and the 3′ end of another nucleotide); (2) it incorporates one or more modified nucleotides (which may comprise a modified base, sugar, or phosphate); and/or (3) a chemical group not normally associated with nucleic acids in nature has been covalently attached to the nucleic acid. Modified nucleic acids include, e.g., locked nucleic acids (in which one or more nucleotides is modified with an extra bridge connecting the 2′ oxygen and 4′ carbon i.e., at least one C-methylene-β-D-ribofuranosyl nucleotide), morpholinos (nucleic acids in which at least some of the nucleobases are bound to morpholine rings instead of deoxyribose or ribose rings and linked through phosphorodiamidate groups instead of phosphates), and peptide nucleic acids (in which the backbone is composed of repeating N-(2-aminoethyl)-glycine units linked by peptide bonds and the nucleobases are linked to the backbone by methylene carbonyl bonds). Modifications may occur anywhere in a nucleic acid. A modified nucleic acid may be modified throughout part or all of its length, may contain alternating modified and unmodified nucleotides or internucleoside linkages, or may contain one or more segments of unmodified nucleic acid and one or more segments of modified nucleic acid. A modified nucleic acid may contain multiple different modifications, which may be of different types. A modified nucleic acid may have increased stability (e.g., decreased susceptibility to spontaneous or nuclease-catalyzed hydrolysis) or altered hybridization properties (e.g., increased affinity or specificity for a target, e.g., a complementary nucleic acid), relative to an unmodified counterpart having the same nucleobase sequence. In some embodiments a modified nucleic acid comprises a modified nucleobase having a label covalently attached thereto. Non-standard nucleotides and other nucleic acid modifications known in the art as being useful in the context of nucleic acid detection reagents, RNA interference (RNAi), aptamer, or antisense-based molecules for research or therapeutic purposes are contemplated for use in various embodiments of the instant invention. See, e.g., The Molecular Probes® Handbook—A Guide to Fluorescent Probes and Labeling Technologies (cited above), Bioconjugate Techniques (cited above), Crooke, S T (ed.) Antisense drug technology: principles, strategies, and applications, Boca Raton: CRC Press, 2008; Kurrcek. J. (ed.) Therapeutic oligonucleotides, RSC biomolecular sciences. Cambridge: Royal Society of Chemistry, 2008, A nucleic acid can be single-stranded, double-stranded, or partially double-stranded. An at least partially double-stranded nucleic acid can have one or more overhangs, e.g., 5′ and/or 3′ overhang(s). Where a nucleic acid sequence is disclosed herein, it should be understood that its complement and double-stranded form is also disclosed.

A “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. In some embodiments, a non-standard, naturally occurring amino acid is found in mammals. For example, ornithine, citrulline, and homocysteine are naturally occurring non-standard amino acids that have important roles in mammalian metabolism. Examples of non-standard amino acids include, e.g., singly or multiply halogenated (e.g., fluorinated) amino acids, D-amino acids, homo-amino acids, N-alkyl amino acids (other than proline), dehydroamino acids, aromatic amino acids (other than histidine, phenylalanine, tyrosine and tryptophan), and α,α disubstituted amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, etc. Modifications may occur anywhere in a polypeptide, e.g., the peptide backbone, the amino acid side-chains and the amino or carboxyl termini. A given polypeptide may contain many types of modifications. Polypeptides may be branched or they may be cyclic, with or without branching. Polypeptides may be conjugated with, encapsulated by, or embedded within a polymer or polymeric matrix, dendrimer, nanoparticle, microparticle, liposome, or the like. Modification may occur prior to or after an amino acid is incorporated into a polypeptide in various embodiments. Polypeptides may, for example, be purified from natural sources, produced in vitro or in vivo in suitable expression systems using recombinant DNA technology (e.g., by recombinant host cells or in transgenic animals or plants), synthesized through chemical means such as conventional solid phase peptide synthesis, and/or methods involving chemical ligation of synthesized peptides (see, e.g., Kent, S., J Pept Sci., 9(9):574-93, 2003 or U.S. Pub. No. 20040115774), or any combination of the foregoing.

As used herein, the term “purified” refers to agents that have been separated from most of the components with which they are associated in nature or when originally generated or with which they were associated prior to purification. In general, such purification involves action of the hand of man. Purified agents may be partially purified, substantially purified, or pure. Such agents may be, for example, at least 50%, 60%, 70%, 75%, 80%, 85%, 90%, 95%, 96%, 97%, 98%, 99%, or more than 99% pure. In some embodiments, a nucleic acid, polypeptide, or small molecule is purified such that it constitutes at least 75%, 80%, 85%, 90%, 95%, 96%, 97%, 98%, 99%, or more, of the total nucleic acid, polypeptide, or small molecule material, respectively, present in a preparation. In some embodiments, an organic substance, e.g., a nucleic acid, polypeptide, or small molecule, is purified such that it constitutes at least 75%, 80%, 85%, 90%, 95%, 96%, 97%, 98%, 99%, or more, of the total organic material present in a preparation. Purity may be based on, e.g., dry weight, size of peaks on a chromatography tracing (GC, HPLC, etc.), molecular abundance, electrophoretic methods, intensity of bands on a gel, spectroscopic data (e.g., NMR), elemental analysis, high throughput sequencing, mass spectrometry, or any art-accepted quantification method. In some embodiments, water, buffer substances, ions, and/or small molecules (e.g., synthetic precursors such as nucleotides or amino acids), can optionally be present in a purified preparation. A purified agent may be prepared by separating it from other substances (e.g., other cellular materials), or by producing it in such a manner to achieve a desired degree of purity. In some embodiments “partially purified” with respect to a molecule produced by a cell means that a molecule produced by a cell is no longer present within the cell, e.g., the cell has been lysed and, optionally, at least some of the cellular material (e.g., cell wall, cell membrane(s), cell organelle(s)) has been removed and/or the molecule has been separated or segregated from at least some molecules of the same type (protein, RNA, DNA, etc.) that were present in the lysate.

The term “RNA interference” (RNAi) encompasses processes in which a molecular complex known as an RNA-induced silencing complex (RISC) silences or “knocks down” gene expression in a sequence-specific manner in, e.g., eukaryotic cells, e.g., vertebrate cells, or in an appropriate in vitro system. RISC may incorporate a short nucleic acid strand (e.g., about 16- about 30 nucleotides (nt) in length) that pairs with and directs or “guides” sequence-specific degradation or translational repression of RNA (e.g., mRNA) to which the strand has complementarity. The short nucleic acid strand may be referred to as a “guide strand” or “antisense strand”. An RNA strand to which the guide strand has complementarity may be referred to as a “target RNA”. A guide strand may initially become associated with RISC components (in a complex sometimes termed the RISC loading complex) as part of a short double-stranded RNA (dsRNA), e.g., a short interfering RNA (siRNA).

As used herein, the term “RNAi agent” encompasses nucleic acids that can be used to achieve RNAi in eukaryotic cells. Short interfering RNA (siRNA), short hairpin RNA (shRNA), and microRNA (miRNA) are examples of RNAi agents. siRNAs typically comprise two separate nucleic acid strands that are hybridized to each other to form a structure that contains a double stranded (duplex) portion at least 15 nt in length, e.g., about 15- about 30 nt long, e.g., between 17-27 nt long, e.g., between 18-25 nt long, e.g., between 19-23 nt long, e.g., 15, 16, 17, 18, 19, 20, 21, 22, 23, 24, 25, 26, 27, 28, 29, or 30 nucleotides. In some embodiments the strands of an siRNA are perfectly complementary to each other within the duplex portion. In some embodiments the duplex portion may contain one or more unmatched nucleotides, e.g., one or more mismatched (non-complementary) nucleotide pairs or bulged nucleotides. In some embodiments either or both strands of an siRNA may contain up to about 1, 2, 3, or 4 unmatched nucleotides within the duplex portion. In some embodiments a strand may have a length of between 15-35 nt, e.g., between 17-29 nt, e.g., 19-25 nt, e.g., 21-23 nt. Strands may be equal in length or may have different lengths in various embodiments. In some embodiments strands may differ by between 1-10 nt in length. A strand may have a 5′ phosphate group and/or a 3′ hydroxyl (—OH) group. Either or both strands of an siRNA may comprise a 3′ overhang of, e.g., about 1-10 nt (e.g., 1-5 nt, e.g., 2 nt). Overhangs may be the same length or different in lengths in various embodiments. In some embodiments an overhang may comprise or consist of deoxyribonucleotides, ribonucleotides, or modified nucleotides or modified ribonucleotides such as 2′-O-methylated nucleotides, or 2′-O-methyl-uridine. An overhang may be perfectly complementary, partly complementary, or not complementary to a target RNA in a hybrid formed by the guide strand and the target RNA in various embodiments. shRNAs are nucleic acid molecules that comprise a stem-loop structure and a length typically between about 40-150 nt, e.g., about 50-100 nt, e.g., 60-80 nt. A “stem-loop structure” (also referred to as a “hairpin” structure) refers to a nucleic acid having a secondary structure that includes a region of nucleotides which are known or predicted to form a double strand (stem portion; duplex) that is linked on one side by a region of (usually) predominantly single-stranded nucleotides (loop portion). Such structures are well known in the art and the term is used consistently with its meaning in the art. A guide strand sequence may be positioned in either arm of the stem, i.e., 5′ with respect to the loop or 3′ with respect to the loop in various embodiments. As is known in the art, the stem structure does not require exact base-pairing (perfect complementarity). Thus, the stem may include one or more unmatched residues or the base-pairing may be exact, i.e., it may not include any mismatches or bulges. In some embodiments the stem is between 15-30 nt, e.g., between 17-29 nt, e.g., 19-25 nt. In some embodiments the stem is between 15-19 nt. In some embodiments a loop sequence may be absent (in which case the termini of the duplex portion may be directly linked). In some embodiments a loop sequence may be at least partly self-complementary. In some embodiments the loop is between 1 and 20 nt in length, e.g., 1-15 nt, e.g., 4-9 nt. The shRNA structure may comprise a 5′ or 3′ overhang. As known in the art, an shRNA may undergo intracellular processing, e.g., by the ribonuclease (RNase) III family enzyme known as Dicer, to remove the loop and generate an siRNA.

Mature endogenous miRNAs are short (typically 18-24 nt, e.g., about 22 nt), single-stranded RNAs that are generated by intracellular processing from larger, endogenously encoded precursor RNA molecules termed miRNA precursors (see, e.g., Bartel, D., Cell. 116(2):281-97 (2004); Bartel D P, Cell. 136(2):215-33 (2009); Winter, J., et al., Nature Cell Biology 11: 228-234 (2009). Artificial miRNA may be designed to take advantage of the endogenous RNAi pathway in order to silence a target RNA of interest.

In some embodiments an RNAi agent is a vector (e.g., an expression vector) suitable for causing intracellular expression of one or more transcripts that give rise to a siRNA, shRNA, or miRNA in the cell. Such a vector may be referred to as an “RNAi vector”. An RNAi vector may comprise a template that, when transcribed, yields transcripts that may form a siRNA (e.g., as two separate strands that hybridize to each other), shRNA, or miRNA precursor (e.g., pri-miRNA or pre-mRNA).

An RNAi agent that contains a strand sufficiently complementary to an RNA of interest so as to result in reduced expression of the RNA of interest (e.g., as a result of degradation or repression of translation of the RNA) in a cell or in an in vitro system capable of mediating RNAi and/or that comprises a sequence that is at least 80%, 90%, 95%, or more (e.g., 100%) complementary to a sequence comprising at least 10, 12, 15, 17, or 19 consecutive nucleotides of an RNA of interest may be referred to as being “targeted to” the RNA of interest. An RNAi agent targeted to an RNA transcript may also considered to be targeted to a gene from which the transcript is transcribed. An RNAi agent may be produced in any of variety of ways in various embodiments. For example, nucleic acid strands may be chemically synthesized (e.g., using standard nucleic acid synthesis techniques) or may be produced in cells or using an in vitro transcription system. Strands may be allowed to hybridize (anneal) in an) appropriate liquid composition (sometimes termed an “annealing buffer”). An RNAi vector may be produced using standard recombinant nucleic acid techniques.

The term “sample” may be used to generally refer to an amount or portion of something. A sample may be a smaller quantity taken from a larger amount or entity; however, a complete specimen may also be referred to as a sample where appropriate. A sample is often intended to be similar to and representative of a larger amount of the entity of which it is a sample. In some embodiments a sample is a quantity of a substance that is or has been or is to be provided for assessment (e.g., testing, analysis, measurement) or use. A sample may be any biological specimen. In some embodiments a sample comprises a body fluid such as blood, cerebrospinal fluid, (CSF), sputum, lymph, mucus, saliva, a glandular secretion, or urine. In some embodiments a sample comprises cells, tissue, or cellular material (e.g., material derived from cells, such as a cell lysate or fraction thereof). A sample may be obtained from (i.e., originates from, was initially removed from) a subject. Methods of obtaining biological samples from subjects are known in the art and include, e.g., tissue biopsy, such as excisional biopsy, incisional biopsy, core biopsy; fine needle aspiration biopsy; surgical excision, brushings; lavage; or collecting body fluids that may contain cells, such as blood, sputum, lymph, mucus, saliva, or urine. A sample is often intended to be similar to and representative of a larger amount of the entity of which it is a sample. A sample of a cell line comprises a limited number of cells of that cell line. A tumor sample is a sample that comprises at least some tumor cells, e.g., at least some tumor tissue. In some embodiments a sample may be obtained from an individual who has been diagnosed with or is suspected of having cancer. In some embodiments a sample is obtained from a tumor, e.g., a solid tumor. In some embodiments a tumor sample is obtained from the interior of a tumor. In some embodiments a tumor sample may comprise some non-tumor tissue or non-tumor cells, in addition to tumor tissue or tumor cells. For example a sample from the edge of a tumor may include some tumor tissue and some non-tumor tissue. A tumor sample may be obtained from a tumor prior to, during, or following removal of the tumor from a subject, or without removing the tumor from the subject. In some embodiments a sample contains at least some intact cells. In some embodiments a sample retains at least some of the microarchitecture of a tissue from which it was removed. A sample may be subjected to one or more processing steps, e.g., after having been obtained from a subject, and/or may be split into one or more portions. For example, in some embodiments a sample comprises plasma or serum obtained from a blood sample that has been processed to obtain such plasma or serum. The term sample encompasses processed samples, portions of samples, etc., and such samples are, where applicable, considered to have been obtained from the subject from whom the initial sample was removed. A sample may be procured directly from a subject, or indirectly, e.g., by receiving the sample from one or more persons who procured the sample directly from the subject, e.g., by performing a biopsy, surgery, or other procedure on the subject. In some embodiments a sample may be assigned an identifier (ID), which may be used to identify the sample as it is transported, processed, analyzed, and/or stored. In some embodiments the sample ID corresponds to the subject from whom the sample originated and allows the sample and/or results obtained by assessing the sample to be matched with the subject. In some embodiments the sample has an identifier affixed thereto. In some embodiments the identifier comprises a bar code.

A “small molecule” as used herein, is an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da, Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

“Specific binding” generally refers to a physical association between a target molecule (e.g., a polypeptide) or complex and a binding agent such as an antibody, aptamer or ligand. The association is typically dependent upon the presence of a particular structural feature of the target such as an antigenic determinant, epitope, binding pocket or cleft, recognized by the binding agent. For example, if an antibody is specific for epitope A, the presence of a polypeptide containing epitope A or the presence of free unlabeled A in a reaction containing both free labeled A and the binding agent that binds thereto, will typically reduce the amount of labeled A that binds to the binding agent. It is to be understood that specificity need not be absolute but generally refers to the context in which the binding occurs. For example, it is well known in the art that antibodies may in some instances cross-react with other epitopes in addition to those present in the target. Such cross-reactivity may be acceptable depending upon the application for which the antibody is to be used. One of ordinary skill in the art will be able to select binding agents, e.g., antibodies, aptamers, or ligands, having a sufficient degree of specificity to perform appropriately in any given application (e.g., for detection of a target molecule). It is also to be understood that specificity may be evaluated in the context of additional factors such as the affinity of the binding agent for the target versus the affinity of the binding agent for other targets, e.g., competitors. If a binding agent exhibits a high affinity for a target molecule that it is desired to detect and low affinity for nontarget molecules, the binding agent will likely be an acceptable reagent. Once the specificity of a binding agent is established in one or more contexts, it may be employed in other contexts, e.g., similar contexts such as similar assays or assay conditions, without necessarily re-evaluating its specificity. In some embodiments specificity of a binding agent can be tested by performing an appropriate assay on a sample expected to lack the target (e.g., a sample from cells in which the gene encoding the target has been disabled or effectively inhibited) and showing that the assay does not result in a signal significantly different to background. In some embodiments, a first entity (e.g., molecule, complex) is said to “specifically bind” to a second entity if it binds to the second entity with substantially greater affinity than to most or all other entities present in the environment where such binding takes place and/or if the two entities bind with an equilibrium dissociation constant, K_{d}, of 10⁻⁴ or less, e.g., 10⁻⁵ M or less, e.g., 10⁻⁶ M or less, 10⁻⁷ M or less, 10⁻⁸ M or less, 10⁻⁹ M or less, or 10⁻¹⁰ M or less. K_{d} can be measured using any suitable method known in the art, e.g., surface plasmon resonance-based methods, isothermal titration calorimetry, differential scanning calorimetry, spectroscopy-based methods, etc. “Specific binding agent” refers to an entity that specifically binds to another entity, e.g., a molecule or molecular complex, which may be referred to as a “target”. “Specific binding pair” refers to two entities (e.g., molecules or molecular complexes) that specifically bind to one another. Examples are biotin-avidin, antibody-antigen, complementary nucleic acids, receptor-ligand, etc.

A “subject” may be any vertebrate organism in various embodiments. A subject may be individual to whom an agent is administered, e.g., for experimental, diagnostic, and/or therapeutic purposes or from whom a sample is obtained or on whom a procedure is performed. In some embodiments a subject is a mammal, e.g. a human, non-human primate, rodent (e.g., mouse, rat, rabbit hamster), ungulate (e.g., ovine, bovine, equine, caprine species), canine, or feline. In some embodiments a subject is an avian. In some embodiments, a human subject is between newborn and 6 months old. In some embodiments, a human subject is between 6 and 24 months old. In some embodiments, a human subject is between 2 and 6, 6 and 12, or 12 and 18 years old. In some embodiments a human subject is between 18 and 30, and 50, 50 and 80, or greater than 80 years old. In some embodiments, a subject is an adult. For purposes hereof a human at least 18 years of age is considered an adult. In some embodiments a subject is an individual who has or may have cancer or is at risk of developing cancer or cancer recurrence.

“Treat”, “treating” and similar terms as used herein in the context of treating a subject refer to providing medical and/or surgical management of a subject. Treatment may include, but is not limited to, administering an agent or composition (e.g., a pharmaceutical composition) to a subject. Treatment is typically undertaken in an effort to alter the course of a disease (which term is used to indicate any disease, disorder, or undesirable condition warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. A therapeutic agent may be administered to a subject who has a disease or is at increased risk of developing a disease relative to a member of the general population. In some embodiments a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur or to reduce the severity of the disease should it occur. The subject may have been identified as being at risk of developing the disease (e.g., at increased risk relative to the general population or as having a risk factor that increases the likelihood of developing the disease.

The term “tumor” as used herein encompasses abnormal growths comprising aberrantly proliferating cells. As known in the art, tumors are typically characterized by excessive cell proliferation that is not appropriately regulated (e.g., that does not respond normally to physiological influences and signals that would ordinarily constrain proliferation) and may exhibit one or more of the following properties: dysplasia (e.g., lack of normal cell differentiation, resulting in an increased number or proportion of immature cells); anaplasia (e.g., greater loss of differentiation, more loss of structural organization, cellular pleomorphism, abnormalities such as large, hyperchromatic nuclei, high nuclear:cytoplasmic ratio, atypical mitoses, etc.); invasion of adjacent tissues (e.g., breaching a basement membrane); and/or metastasis. In certain embodiments a tumor is a malignant tumor, also referred to herein as a “cancer”. Malignant tumors have a tendency for sustained growth and an ability to spread, e.g., to invade locally and/or metastasize regionally and/or to distant locations, whereas benign tumors often remain localized at the site of origin and are often self-limiting in terms of growth. The term “tumor” includes malignant solid tumors (e.g., carcinomas, sarcomas) and malignant growths in which there may be no detectable solid tumor mass (e.g., certain hematologic malignancies). The term “cancer” is generally used interchangeably with “tumor” herein and/or to refer to a disease characterized by one or more tumors, e.g., one or more malignant or potentially malignant tumors. Cancer includes, but is not limited to: breast cancer; biliary tract cancer; bladder cancer; brain cancer (e.g., glioblastomas, medulloblastomas); cervical cancer; choriocarcinoma; colon cancer; endometrial cancer; esophageal cancer; gastric cancer; hematological neoplasms including acute lymphocytic leukemia and acute myelogenous leukemia; T-cell acute lymphoblastic leukemia/lymphoma; hairy cell leukemia; chronic lymphocytic leukemia, chronic myelogenous leukemia, multiple myeloma; adult T-cell leukemia/lymphoma; intraepithelial neoplasms including Bowen's disease and Paget's disease; liver cancer; lung cancer; lymphomas including Hodgkin's disease and lymphocytic lymphomas; neuroblastoma; melanoma, oral cancer including squamous cell carcinoma; ovarian cancer including ovarian cancer arising from epithelial cells, stromal cells, germ cells and mesenchymal cells; neuroblastoma, pancreatic cancer; prostate cancer; rectal cancer; sarcomas including angiosarcoma, gastrointestinal stromal tumors, leiomyosarcoma, rhabdomyosarcoma, liposarcoma, fibrosarcoma, and osteosarcoma; renal cancer including renal cell carcinoma and Wilms tumor; skin cancer including basal cell carcinoma and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullary carcinoma. It will be appreciated that a variety of different tumor types can arise in certain organs, which may differ with regard to, e.g., clinical and/or pathological features and/or molecular markers. Tumors arising in a variety of different organs are discussed, e.g., in DeVita, supra or in the WHO Classification of Tumours series, 4^{th} ed, or 3^{rd} ed (Pathology and Genetics of Tumours series), by the International Agency for Research on Cancer (IARC), WHO Press, Geneva, Switzerland, all volumes of which are incorporated herein by reference.

A “variant” of a particular polypeptide or polynucleotide has one or more alterations (e.g., additions, substitutions, and/or deletions) with respect to the polypeptide or polynucleotide, which may be referred to as the “original polypeptide” or “original polynucleotide”, respectively. An addition may be an insertion or may be at either terminus. A variant may be shorter or longer than the original polypeptide or polynucleotide. The term “variant” encompasses “fragments”. A “fragment” is a continuous portion of a polypeptide or polynucleotide that is shorter than the original polypeptide. In some embodiments a variant comprises or consists of a fragment. In some embodiments a fragment or variant is at least 20%, 30%, 40%, 50%, 60%, 70%, 80%, 90%, 95%, 96%, 97%, 98%, 99%, or more as long as the original polypeptide or polynucleotide. A fragment may be an N-terminal, C-terminal, or internal fragment. In some embodiments a variant polypeptide comprises or consists of at least one domain of an original polypeptide. In some embodiments a variant polynucleotide hybridizes to an original polynucleotide under stringent conditions, e.g., high stringency conditions, for sequences of the length of the original polypeptide. In some embodiments a variant polypeptide or polynucleotide comprises or consists of a polypeptide or polynucleotide that is at least 50%, 60%, 70%, 80%, 90%, 95%, 96%, 97%, 98%, 99%, or more identical in sequence to the original polypeptide or polynucleotide over at least 20%, 30%, 40%, 50%, 60%, 70%, 80%, 90%, 95%, 96%, 97%, 98%, 99%, or 100% of the original polypeptide or polynucleotide. In some embodiments a variant polypeptide comprises or consists of a polypeptide that is at least 50%, 60%, 70%, 80%, 90%, 95%, 96%, 97%, 98%, 99%, or more identical in sequence to the original polypeptide over at least 20%, 30%, 40%, 50%, 60%, 70%, 80%, 90%, 95%, 96%, 97%, 98%, 99%, or 100% of the original polypeptide, with the proviso that, for purposes of computing percent identity, a conservative amino acid substitution is considered identical to the amino acid it replaces. In some embodiments a variant polypeptide comprises or consists of a polypeptide that is at least 50%, 60%, 70%, 80%, 90%, 95%, 96%, 97%, 98%, 99%, or more identical to the original polypeptide over at least 20%, 30%, 40%, 50%, 60%, 70%, 80%, 90%, 95%, 96%, 97%, 98%, 99%, or 100% of the original polypeptide, with the proviso that any one or more amino acid substitutions (up to the total number of such substitutions) may be restricted to conservative substitutions. In some embodiments a percent identity is measured over at least 100; 200; 300; 400; 500; 600; 700; 800; 900; 1,000; 1,200; 1,500; 2,000; 2,500; 3,000; 3,500; 4,000; 4,500; or 5,000 amino acids. In some embodiments the sequence of a variant polypeptide comprises or consists of a sequence that has N amino acid differences with respect to an original sequence, wherein N is any integer between 1 and 10 or between 1 and 20 or any integer up to 1%, 2%, 5%, or 10% of the number of amino acids in the original polypeptide, where an “amino acid difference” refers to a substitution, insertion, or deletion of an amino acid. In some embodiments a difference is a conservative substitution. Conservative substitutions may be made, e.g., on the basis of similarity in side chain size, polarity, charge, solubility, hydrophobicity, hydrophilicity and/or the amphipathic nature of the residues involved. In some embodiments, conservative substitutions may be made according to Table A, wherein amino acids in the same block in the second column and in the same line in the third column may be substituted for one another other in a conservative substitution. Certain conservative substitutions are substituting an amino acid in one row of the third column corresponding to a block in the second column with an amino acid from another row of the third column within the same block in the second column.

TABLE A

| | | |
|---|---|---|
| Aliphatic | Non-polar | G A P |
| | | I L V |
| | Polar - uncharged | C S T M |
| | | N Q |
| | Polar - charged | D E |
| | | K R |
| Aromatic | | H F W Y |

In some embodiments, proline (P) is considered to be in an individual group. In some embodiments, cysteine (C) is considered to be in an individual group. In some embodiments, proline (P) and cysteine (C) are each considered to be in an individual group. Within a particular group, certain substitutions may be of particular interest in certain embodiments, e.g., replacements of leucine by isoleucine (or vice versa), serine by threonine (or vice versa), or alanine by glycine (or vice versa).

In some embodiments a variant is a functional variant, i.e., the variant at least in part retains at least one activity of the original polypeptide or polynucleotide. In some embodiments a variant at least in part retains more than one or substantially all known biologically significant activities of the original polypeptide or polynucleotide. An activity may be, e.g., a catalytic activity, binding activity, ability to perform or participate in a biological function or process, etc: In some embodiments an activity of a variant may be at least 10%, 20%, 30%, 40%, 50%, 60%, 70%, 80%, 90%, 95%, or more, of the activity of the original polypeptide or polynucleotide, up to approximately 100%, approximately 125%, or approximately 150% of the activity of the original polypeptide or polynucleotide, in various embodiments. In some embodiments a variant, e.g., a functional variant, comprises or consists of a polypeptide at least 95%, 96%, 97%, 98%, 99%, 99.5% or 100% identical to an original polypeptide or polynucleotide over at least 70%, 75%, 80%, 85%, 90%, 95%, 96%, 97%, 98%, or 99% or 100% of the original polypeptide or polynucleotide. In some embodiments an alteration, e.g., a substitution or deletion, e.g., in a functional variant, does not alter or delete an amino acid or nucleotide that is known or predicted to be important for an activity, e.g., a known or predicted catalytic residue or residue involved in binding a substrate or cofactor. In some embodiments nucleotide(s), amino acid(s), or region(s) exhibiting lower degrees of conservation across species as compared with other amino acids or regions may be selected for alteration. Variants may be tested in one or more suitable assays to assess activity.

A “vector” may be any of a number of nucleic acid molecules or viruses or portions thereof that are capable of mediating entry of, e.g., transferring, transporting, etc., a nucleic acid of interest between different genetic environments or into a cell. The nucleic acid of interest may be linked to, e.g., inserted into, the vector using, e.g., restriction and ligation. Vectors include, for example, DNA or RNA plasmids, cosmids, naturally occurring or modified viral genomes or portions thereof, nucleic acids that can be packaged into viral capsids, mini-chromosomes, artificial chromosomes, etc. Plasmid vectors typically include an origin of replication (e.g., for replication in prokaryotic cells). A plasmid may include part or all of a viral genome (e.g., a viral promoter, enhancer, processing or packaging signals, and/or sequences sufficient to give rise to a nucleic acid that can be integrated into the host cell genome and/or to give rise to infectious virus). Viruses or portions thereof that can be used to introduce nucleic acids into cells may be referred to as viral vectors. Viral vectors include, e.g., adenoviruses, adeno-associated viruses, retroviruses (e.g., lentiviruses), vaccinia virus and other poxviruses, herpesviruses (e.g., herpes simplex virus), and others. Viral vectors may or may not contain sufficient viral genetic information for production of infectious virus when introduced into host cells, i.e., viral vectors may be replication-competent or replication-defective. In some embodiments, e.g., where sufficient information for production of infectious virus is lacking, it may be supplied by a host cell or by another vector introduced into the cell, e.g., if production of virus is desired. In some embodiments such information is not supplied, e.g., if production of virus is not desired. A nucleic acid to be transferred may be incorporated into a naturally occurring or modified viral genome or a portion thereof or may be present within a viral capsid as a separate nucleic acid molecule. A vector may contain one or more nucleic acids encoding a marker suitable for identifying and/or selecting cells that have taken up the vector. Markers include, for example, various proteins that increase or decrease either resistance or sensitivity to antibiotics or other agents (e.g., a protein that confers resistance to an antibiotic such as puromycin, hygromycin or blasticidin), enzymes whose activities are detectable by assays known in the art (e.g., β-galactosidase or alkaline phosphatase), and proteins or RNAs that detectably affect the phenotype of cells that express them (e.g., fluorescent proteins). Vectors often include one or more appropriately positioned sites for restriction enzymes, which may be used to facilitate insertion into the vector of a nucleic acid, e.g., a nucleic acid to be expressed. An expression vector is a vector into which a desired nucleic acid has been inserted or may be inserted such that it is operably linked to regulatory elements (also termed “regulatory sequences”, “expression control elements”, or “expression control sequences”) and may be expressed as an RNA transcript (e.g., an mRNA that can be translated into protein or a noncoding RNA such as an shRNA or miRNA precursor). Expression vectors include regulatory sequence(s), e.g., expression control sequences, sufficient to direct transcription of an operably linked nucleic acid under at least some conditions; other elements required or helpful for expression may be supplied by, e.g., the host cell or by an in vitro expression system. Such regulatory sequences typically include a promoter and may include enhancer sequences or upstream activator sequences. In some embodiments a vector may include sequences that encode a 5′ untranslated region and/or a 3′ untranslated region, which may comprise a cleavage and/or polyadenylation signal. In general, regulatory elements may be contained in a vector prior to insertion of a nucleic acid whose expression is desired or may be contained in an inserted nucleic acid or may be inserted into a vector following insertion of a nucleic acid whose expression is desired. As used herein, a nucleic acid and regulatory element(s) are said to be “operably linked” when they are covalently linked so as to place the expression or transcription of the nucleic acid under the influence or control of the regulatory element(s). For example, a promoter region would be operably linked to a nucleic acid if the promoter region were capable of effecting transcription of that nucleic acid. One of ordinary skill in the art will be aware that the precise nature of the regulatory sequences useful for gene expression may vary between species or cell types, but may in general include, as appropriate, sequences involved with the initiation of transcription, RNA processing, or initiation of translation. The choice and design of an appropriate vector and regulatory element(s) is within the ability and discretion of one of ordinary skill in the art. For example, one of skill in the art will select an appropriate promoter (or other expression control sequences) for expression in a desired species (e.g., a mammalian species) or cell type. A vector may contain a promoter capable of directing expression in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as EF1alpha, ubiquitin (e.g., ubiquitin B or C), globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, a promoter that ordinarily directs transcription by a eukaryotic RNA polymerase I (a “pol I promoter”), e.g., (a U6, H1, 7SK or tRNA promoter or a functional variant thereof) may be used. In some embodiments, a promoter that ordinarily directs transcription by a eukaryotic RNA polymerase II (a “pol II promoter”) or a functional variant thereof is used. In some embodiments, a promoter that ordinarily directs transcription by a eukaryotic RNA polymerase III promoter, e.g., a promoter for transcription of ribosomal RNA (other than 5S rRNA) or a functional variant thereof is used. One of ordinary skill in the art will select an appropriate promoter for directing transcription of a sequence of interest. Examples of expression vectors that may be used in mammalian cells include, e.g., the pcDNA vector series, pSV2 vector series, pCMV vector series, pRSV vector series, pEF1 vector series, Gateway® vectors, etc. Examples of virus vectors that may be used in mammalian cells include, e.g., adenoviruses, adeno-associated viruses, poxviruses such as vaccinia viruses and attenuated poxviruses, retroviruses (e.g., lentiviruses), Semliki Forest virus, Sindbis virus, etc. In some embodiments, regulatable (e.g., inducible or repressible) expression control element(s), e.g., a regulatable promoter, is/are used so that expression can be regulated, e.g., turned on or increased or turned off or decreased. For example, the tetracycline-regulatable gene expression system (Gossen & Bujard, Proc. Natl. Acad. Sci. 89:5547-5551, 1992) or variants thereof (see, e.g., Allen, N, et al. (2000) Mouse Genetics and Transgenics: 259-263; Urlinger, S, et al. (2000). Proc. Natl. Acad. Sci. U.S.A. 97 (14): 7963-8; Zhou, X., et al (2006). Gene Ther. 13 (19): 1382-1390 for examples) can be employed to provide inducible or repressible expression. Other inducible/repressible systems may be used in various embodiments. For example, expression control elements that can be regulated by small molecules such as artificial or naturally occurring hormone receptor ligands (e.g., steroid receptor ligands such as naturally occurring or synthetic estrogen receptor or glucocorticoid receptor ligands), tetracycline or analogs thereof, metal-regulated systems (e.g., metallothionein promoter) may be used in certain embodiments. In some embodiments, tissue-specific or cell type specific regulatory element(s) may be used, e.g., in order to direct expression in one or more selected tissues or cell types. In some embodiments a vector capable of being stably maintained and inherited as an episome in mammalian cells (e.g., an Epstein-Barr virus-based episomal vector) may be used. In some embodiments a vector may comprise a polynucleotide sequence that encodes a polypeptide, wherein the polynucleotide sequence is positioned in frame with a nucleic acid inserted into the vector so that an N- or C-terminal fusion is created. In some embodiments the polypeptide encoded by the polynucleotide sequence may be a targeting peptide. A targeting peptide may comprise a signal sequence (which directs secretion of a protein) or a sequence that directs the expressed protein to a specific organelle or location in the cell such as the nucleus or mitochondria. In some embodiments the polypeptide comprises a tag. A tag may be useful to facilitate detection and/or purification of a protein that contains it. Examples of tags include polyhistidine-tag (e.g., 6×-His tag), glutathione-S-transferase, maltose binding protein, NUS tag, SNUT tag, Strep tag, epitope tags such as V5, HA, Myc, or FLAG. In some embodiments a protease cleavage site is located in the region between the protein encoded by the inserted nucleic acid and the polypeptide, allowing the polypeptide to be removed by exposure to the protease.

### II. MCT1 Expression as a Biomarker for Sensitivity to 3-BrPA

Glycolysis is a metabolic pathway that converts glucose (C₆H₁₂O₆) into pyruvate, CH₃COCOO⁻+H⁺. The free energy released in this process is used to form the high-energy compound ATP (adenosine triphosphate) and NADH (reduced nicotinamide adenine dinucleotide. Upregulation of glycolysis is a common metabolic alteration in cancer cells. Malignant, rapidly growing tumor cells may have glycolytic rates many times higher than those of their normal counterparts, even if oxygen is plentiful. Many solid tumors are characterized by a relatively low oxygen tension at least in some portions of the tumor due to limited blood supply. Without wishing to be bound by any theory, a high glycolytic rate may, among other things, allow tumor cells to survive and thrive under conditions of hypoxia.

The increased utilization of glycolysis by tumor cells has motivated the development of drug candidates that target this metabolic pathway. 3-bromopyruvic acid (3-BrPA) is an anticancer drug candidate that has cytotoxic effects and decreases cellular energy levels by inhibiting glycolysis (Ko et al. (2001); Can Lett 173:83-91; Geschwind, J F, et al., (2002); Cancer Res; 62(14):3909-13 Ko et al. (2004); Biochem Biophys Res Commun 324:269-275. 3-BrPA has been shown by others to be capable of inhibiting several glycolytic (16-18) and non-glycolytic enzymes (19-23).

The structure of 3-bromopyruvic acid (3-BrPA) is shown below:

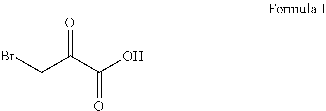

As known in the art, the carboxylate (COO⁻) anion of 3-bromopyruvic acid is 3-bromopyruvate (3-BP). Unless otherwise indicated, the terms 3-bromopyruvic acid and 3-bromopyruvate, and the abbreviations 3-BrPA and 3-BP, are used interchangeably herein, as common in the art, and encompass embodiments in which the acid form (Formula I) and/or its conjugate base (the carboxylate anion) are present. It will be understood that the proportion of 3-bromopyruvic acid relative to 3-bromopyruvate (its conjugate base) in a composition will vary with pH. In some embodiments 3-bromopyruvate is provided as a salt.

Monocarboxylates such as pyruvate, lactate, and ketone bodies (acetoacetate and hydroxybutyrate) play important roles in carbohydrate, fat, and amino acid metabolism. Transport of these compounds across the plasma membrane of cells occurs via proton-linked monocarboxylate transporters (MCTs). MCT1, MCT2, MCT3, and MCT4 are confirmed proton-linked MCTs in mammals and have distinct substrate and inhibitor affinities. They are part of the larger SLC16 family of solute carriers, also known as the MCT family, which has 14 members that share conserved sequence motifs.

In some aspects, the present invention relates to the discovery that monocarboxylate transporter 1 (MCT1) is the main determinant of cell sensitivity to 3-BrPA. As described in the Examples, Applicants preformed a genome-wide loss-of-function screen using insertional mutagenesis in near-haploid mammalian cells in order to identify resistance mechanisms to 3-BrPA. Loss of function of the gene encoding MCT1 was found to confer resistance to 3-BrPA-induced cell death. Furthermore, among 20,000 mRNAs examined, MCT1 mRNA level was found to be the best predictor of 3-BrPA sensitivity across a large set of cancer cell lines. MCT1 was shown to be necessary and sufficient for 3-BrPA uptake by a variety of different cancer cell lines, and its expression was most elevated in highly glycolytic cancer cells. In addition, forced MCT1 expression in 3-BrPA-resistant cancer cells was found to be sufficient to sensitize tumor xenografts to 3-BrPA treatment in vivo. Thus, results described herein demonstrate that MCT1 is the main determinant of 3-BrPa uptake and sensitivity and establish that MCT1 expression is a biomarker for 3-BrPA sensitivity.

The gene encoding MCT1 is also known as SLC16A1. MCT1 associates with a protein named basigin (BSG, also known as CD147 or EMMPRIN), which acts as a chaperone to escort MCT1 to the plasma membrane. Further details regarding MCT1 and various other SLC16 family members is found in Halestrap, A. P. and Meredith, D. (2004); Pflugers Arch. 447, 619-628; Halestrap, A. P. and Price, N. T. (1999); Biochem. J. 343, 281-299; Meredith, D. and Christian, H. C. (2008); Xenobiotica 38, 1072-1106; Halestrap, A P, IUBMB Life, 64(1): 1-9; Halestrap, A P and Wilson, M C; (2012); IUBMB Life, 64(2): 109-119. Genomic, mRNA, and polypeptide sequences of MCT1 and other genes and gene products of interest herein are known in the art and are available in databases such as the National Center for Biotechnology Information (ncbi.nih.gov) or Universal Protein Resource (uniprot.org) databases, e.g., GenBank, RefSeq, Gene, UniProtKB/SwissProt, UniProtKB/Trembl, and the like. Sequence information may be employed, for example, in generating or testing detection reagents of use in methods described herein.

Table 1 lists the NCBI Gene IDs for the human genes encoding MCT1, MCT2, MCT3, MCT4, and BSG and NCBI Reference Sequence accession numbers for human MCT1, MCT2, MCT3, MCT4, and BSG mRNAs and polypeptides respectively. Multiple mRNA transcript variants have been identified for MCT1, MCT2, and MCT4. Transcript variants for MCT1 differ in their 5′ untranslated region but encode the same protein. Human MCT1 protein has the following sequence:

(SEQ ID NO: 1)

MPPAVGGPVGYTPPDGGWGWAVVIGAFISIGFSYAFPKSITVFFKEIEGI
FHATTSEVSWISSIMLAVMYGGGPISSILVNKYGSRIVMIVGGCLSGCGL
IAASFCNTVQQLYVCIGVIGGLGLAFNLNPALTMIGKYFYKRRPLANGLA
MAGSPVFLCTLAPLNQVFFGIFGWRGSFLILGGLLLNCCVAGALMRPIGP
KPTKAGKDKSKASLEKAGKSGVKKDLHDANTDLIGRHPKQEKRSVFQTIN
QFLDLTLFTHRGFLLYLSGNVIMFFGLFAPLVFLSSYGKSQHYSSEKSAF
LLSILAFVDMVARPSMGLVANTKPIRPRIQYFFAASVVANGVCHMLAPLS
TTYVGFCVYAGFFGFAFGWLSSVLFETLMDLVGPQRFSSAVGLVTIVECC
PVLLGPPLLGRLNDMYGDYKYTYWACGVVLIISGIYLFIGMGINYRLLAK
EQKANEQKKESKEEETSIDVAGKPNEVTKAAESPDQKDTDGGPKEEESP
V.

Any one or more MCT1 transcript variants may be detected in various embodiments. Transcript variant 1 of BSG is the longest transcript and encodes the longest isoform. Transcript variant 2 of BSG lacks an alternate in-frame exon compared to variant 1 such that the resulting isoform (2) has the same N- and C-termini but is shorter compared to isoform 1. Transcript variant 3 differs in the 5′ UTR and coding sequence compared to variant 1 such that the resulting isoform (3) is shorter at the N-terminus compared to isoform 1. Transcript variant 4 differs in the 5′ UTR and coding sequence compared to variant 1 such that the resulting isoform (4) has a shorter and distinct N-terminus compared to isoform 1. Any one or more BSG transcript variants or isoforms may be detected in various embodiments. Detection of particular variants or isoforms may be accomplished using suitable detection reagents and/or by performing an assay under appropriate conditions. For example, antibodies that specifically bind to one, more than one, or all isoforms may be used. Probes, primers, and/or hybridization conditions can be selected such that a probe or primer will hybridize with one, more than one, or all variants.

TABLE 1
Human Gene IDs, Symbols, Names, and NCBI RefSeq Accession
Numbers*

| | Gene Official | | |
|---|---|---|---|
| Gene ID | Symbol and Name | mRNA | Protein |
| MCT1 | SLC16A1/solute | NM_001166496.1 | NP_001159968.1 |
| Gene | carrier family 16, | NM_003051.3 | NP_003042.3 |
| ID: 6566 | member 1 | | |
| | (monocarboxylic | | |
| | acid transporter | | |
| | 1) | | |
| MCT2 | SLC16A7/solute | NM_001270622.1 | NP_001257551.1 |
| Gene | carrier family 16, | NM_001270623.1 | NP_001257552.1 |
| ID: 9194 | member 7 | NM_004731.4 | NP_004722.2 |
| | (monocarboxylic | | |
| | acid transporter 2) | | |
| MCT3 | SLC16A8/solute | NM_013356.2 | NP_037488.2 |
| Gene | carrier family 16, | | |
| ID: 23539 | member 8 | | |
| | (monocarboxylic | | |
| | acid transporter 3) | | |
| MCT4 | SLC16A3/solute | NM_001042422.2 | NP_001035887.1 |
| Gene | carrier family 16, | NM_001042423.2 | NP_001035888.1 |
| ID: 9123 | member 3 | NM_001206950.1 | NP_001193879.1 |
| | (monocarboxylic | NM_001206951.1 | NP_001193880.1 |
| | acid transporter 4) | NM_001206952.1 | NP_001193881.1 |
| | | NM_004207.3 | NP_004198.1 |
| basigin | BSG/basigin | NM_001728.3 | NP_001719.2 |
| Gene | | NM_198589.2 | NP_940991.1 |
| ID: 682 | | NM_198590.2 | NP_940992.1 |
| | | NM_198591.2 | NP_940993.1 |

*numeral following the decimal point in accession numbers is the version number.

In some aspects, the present disclosure encompasses the insight that MCT1-mediated transport can be used to deliver toxic agents to glycolytic tumor cells or tumors in order to inhibit survival and/or proliferation of tumor cells. MCT1-mediated transport of such agents thus offers a promising therapeutic strategy for treatment of cancer. Toxic agents that are taken up by MCT1 may be of particular use to treat tumors that express increased levels of MCT1. MCT1 expression can be used as a biomarker for sensitivity to such agents, e.g., to identify tumors that have an increased likelihood of being sensitive to such molecules. As used herein, a “toxic agent” refers to an agent that is capable of inhibiting survival or proliferation of at least some mammalian cells exposed to the agent or is otherwise deleterious to such cells. A toxic agent may, for example, inhibit the activity of one or more biological pathways, processes, proteins, or other cellular components that promotes or is essential for cell viability or proliferation.

In some aspects, the invention provides methods that comprise assessing MCT1 expression for purposes of tumor classification, treatment selection, and/or predicting tumor responsiveness to 3-BrPA or an analog thereof. In some aspects, described herein are methods of classifying a tumor cell, tumor cell line, or tumor according to predicted sensitivity to 3-bromopyruvate (3-BrPA) or an analog thereof. In some embodiments the methods comprise: (a) assessing the level of expression of the MCT1 gene in a tumor cell, tumor cell line, or tumor, wherein an increased level of MCT1 gene product is correlated with increased sensitivity to the compound; and (b) classifying the tumor cell, tumor cell line, or tumor with respect to predicted sensitivity to the compound based at least in part on the level of expression of the MCT1 gene. In some embodiments assessing expression of the MCT1 gene in a tumor comprises assessing expression of the MCT1 gene in one or more samples obtained from the tumor. In certain embodiments expression of MCT1, e.g., increased (elevated) expression of MCT1, e.g., high expression of MCT1, identifies tumor cells or tumors that are sensitive to 3-BrPA or a 3-BrPA analog. In certain embodiments increased expression of MCT1, e.g., high expression of MCT1, is used to identify subjects with cancer who are candidates for treatment with 3-BrPA or a 3-BrPA analog. In some embodiments, a measurement of MCT expression is used to establish whether a subject in need of treatment for cancer will likely respond (or not respond) to treatment with 3-BrPA or an analog thereof. In certain embodiments, a tumor is determined to have increased expression of MCT1, e.g., high expression of MCT1, and a subject in need of treatment for the tumor is treated with 3-BrPA or an analog thereof. The methods may similarly be used to classify a tumor cell, tumor cell line, or tumor according to predicted sensitivity to other toxic agents taken up via MCT1.

In some embodiments assessing the level of expression of the MCT1 gene comprises determining the level of an MCT1 gene product in a tumor cell, tumor cell line, tumor or sample obtained from a tumor. In some embodiments the method comprises comparing the level of an MCT1 gene product in a tumor cell, tumor cell line, tumor, or sample with a reference level, wherein if the level of the MCT1 gene product in the tumor cell, tumor cell line, tumor, or sample is greater than the reference level, the tumor cell, tumor cell line, or tumor is classified as having an increased likelihood of being sensitive to the compound than if the level is not greater than the reference level.

In some aspects, described herein are methods of predicting the likelihood that a tumor cell, tumor cell line, or tumor, is sensitive to a compound, wherein the compound is 3-bromopyruvate (3-BrPA) or an analog thereof, the method comprising: (a) assessing expression of the MCT1 gene by the tumor cell, tumor cell line, or tumor; and (b) generating a prediction of the likelihood that the tumor cell, tumor cell line, or tumor, is sensitive to the compound, wherein if the tumor cell, tumor cell line, or tumor, has increased expression of the MCT1 gene, the tumor cell, tumor cell line, or tumor, is predicted to have increased likelihood of being sensitive to the compound. In some embodiments the tumor cell, tumor cell line, or tumor exhibits high expression of MCT1. In some embodiments, assessing expression of the MCT1 gene comprises determining the level of an MCT1 gene product in the tumor cell, tumor cell line, tumor, or a sample obtained therefrom. In some embodiments the method comprises comparing the level of MCT1 gene product with a reference level of the MCT1 gene product, wherein if the level is greater than the reference level, the tumor cell, tumor cell line, or tumor has increased expression of MCT1. In some embodiments the method comprises comparing the level of MCT1 gene product with a reference level of the MCT1 gene product, wherein if the level is greater than the reference level, the tumor cell, tumor cell line, or tumor has increased likelihood of being sensitive to the compound than if the level determined in (a) is not greater than the reference level.

In some aspects, described herein are methods of determining whether a subject in need of treatment for a tumor is a candidate for treatment with 3-BrPA or an analog thereof, the methods comprising: (a) determining whether the tumor expresses the MCT1 gene; and (b) identifying the subject as a candidate for treatment with 3-BrPA or an analog thereof if the tumor expresses the MCT1 gene. In some embodiments the method comprises identifying the subject as a candidate for treatment with 3Br-PA or analog thereof if the tumor has increased expression of MCT1. In general, a subject is a candidate for treatment with an agent if there is sufficient likelihood that the tumor will respond to the agent to justify the risk (e.g., potential side effects) associated with the agent within the judgment of a person of ordinary skill in the art, e.g., a physician such as an oncologist. For example, if a subject has a tumor that lacks MCT1 expression, the subject would not be a candidate for treatment with 3-BrPA since the tumor would be expected to be insensitive to the compound, while if the tumor exhibits increased MCT1 expression, the subject would be a candidate for treatment with 3-BrPA. It will be understood that MCT1 expression level may be used together with one or more additional criteria to determine whether the subject should be treated with 3-BrPA. Such criteria may include, for example, predicted sensitivity or previous response of the tumor to other therapies. In some embodiments MCT1 expression level is used in a clinical decision support system (i.e., a computer program product designed to assist physicians and other health professionals with decision making tasks), optionally together with additional information about the tumor and/or subject, to select or assist a health care provider in selecting a treatment for the subject.

It will be understood that the terms “sensitive” or “resistant” as used herein in regard to sensitivity or resistance to agents or conditions, generally refers to the extent to which a cell, e.g., a tumor cell, or tumor is susceptible to or able to withstand the potential inhibitory effects of an agent or condition to which it is exposed on survival and/or proliferation. For example, tumor cell(s) may be considered sensitive if killed or rendered nonproliferative by an agent, while they may be considered resistant if able to survive and proliferate in the presence of the agent. It will be understood that sensitivity or resistance may at least depend on concentration of an agent, duration of exposure, etc. In some embodiments the level of sensitivity of a cell to an agent may be determined by contacting cells with the agent, e.g., by culturing cells in culture medium containing the agent, and measuring cell survival or proliferation after a suitable time period. Any suitable method of assessing cell survival or proliferation may be used. Examples are described herein, e.g., in Section III. In some embodiments a classification according to predicted 3-BrPA sensitivity based on MCT1 expression correlates with sensitivity as determined by contacting a cell with 3-BrPA and measuring cell survival or proliferation or using a method described in Section III.

In some embodiments tumor cells are classified as sensitive or resistant to 3-BrPA or classified as having an increased or decreased likelihood of being sensitive or resistant to 3-BrPA. In some embodiments tumor cells are considered sensitive to 3-BrPA if the IC₅₀ of 3-BrPA is below about 20 μm, e.g., between 1 μm and 5 μm, between 5 μm and 10 μm, or between 10 μm 3-BrPA and 20 μm. In some embodiments tumor cells are considered sensitive to 3-BrPA if the IC₉₀ of 3-BrPA is below about 40 μm, e.g., between 10 μm and 20 μm, between 20 μm and 30 μm, or between 30 μm 3-BrPA and 40 μm. In some embodiments tumor cells are considered resistant to 3-BrPA if the IC₅₀ of 3-BrPA is above about 150 μm, e.g., above 175 μm, above 200 μm, above 250 μm, above 300 μm, above 350 μm, or above 400 μm, e.g., between 200 μm and 500 μm or between 500 μm and 1 mm.

In some embodiments, the level of MCT1 expression is used to predict in vivo tumor sensitivity to 3-BrPA, e.g., to identify a tumor or subject having increased likelihood of responding to treatment with 3-BrPA or to predict the likelihood that a tumor or subject will respond to treatment with 3-BrPA. Methods and criteria that may be employed for evaluating tumor progression, response to treatment, and outcomes are known in the art and may include objective measurements (e.g., anatomical tumor burden) and criteria, clinical evaluation of symptoms, or combinations thereof. Examples are described herein, e.g., in Section III. For example, imaging may be used to detect or assess number, size or metabolic activity of tumors (local or metastatic). In some embodiments a classification according to predicted 3-BrPA sensitivity based on MCT1 expression correlates with sensitivity as determined by measuring tumor response using a method described in Section III. In some embodiments a tumor is considered sensitive to an agent if a response can be obtained when the agent is administered to a subject using dose(s) that can be reasonably tolerated by the subject, while if a response is not obtained within the tolerated dose range, the tumor is considered resistant to the agent.

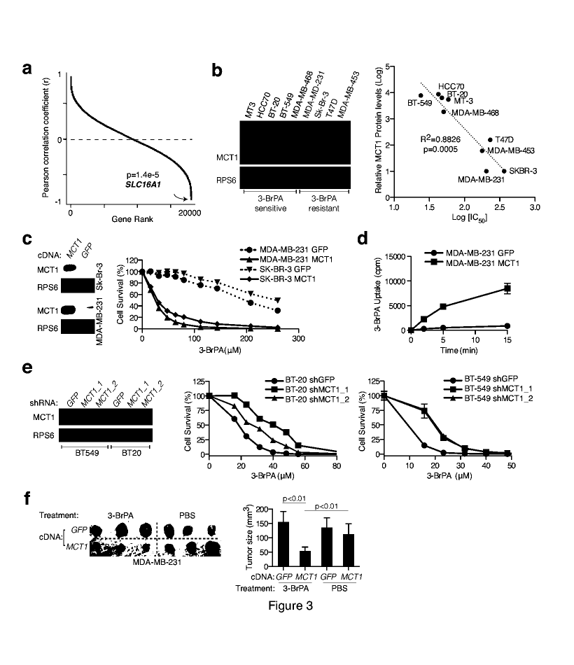

Examples of tumor cell lines that are sensitive or resistant to 3-BrPA are described herein. For example, BT-549, HCC-70, BT-20, MDA-MB-468 and MT-3 cells express increased levels of MCT1 and are sensitive to 3-BrPA (FIGS. 3b and 3c), whereas SK-BR-3, MDA-MB-231, MDA-MB-453, and T47D cells express low levels of MCT1 and are resistant to 3-BrPA (FIGS. 3b and 3c). In some embodiments an increased level of an MCT1 gene product (e.g., MCT1 protein) is a level at least about 2.5, 3.0, 3.5, or 4.0-fold higher than that in SK-BR-3 cells. In some embodiments an increased level of an MCT1 gene product (e.g., MCT1 protein) is a level at least equal to about the level in MDA-MB-468 cells. In some embodiments an increased level of an MCT1 gene product, e.g., MCT1 protein, is a high level, e.g., a level at or above the level in BT-549, HCC-70, BT-20, or MT-3 cells. In some embodiments a high level of an MCT1 gene product, e.g., MCT1 protein, is a level at least equal to the level of an RPS6 gene product, e.g., RPS6 protein. In some embodiments a low level of an MCT1 gene product (e.g., MCT1 protein) is a level below about the level in SK-BR-3, MDA-MB-231, MDA-MB-453, or T47D cells. Thus in some embodiments, a tumor cell, tumor cell line, or tumor is classified as having an increased likelihood of being sensitive to 3-BrPA if it expresses MCT1 at a level equal to or greater than the level at which BT-549, HCC-70, BT-20, MT-3, and/or MDA-MB-468 cells express MCT1. In some embodiments, a tumor cell, tumor cell line, or tumor is classified as having a decreased likelihood of being sensitive to 3-BrPA if it expresses MCT1 at a level equal to or below the level at which SK-BR-3, MDA-MB-231, MDA-MB-453, or T47D cells express MCT1.

Expression of the MCT1 gene can be assessed using any of a variety of methods. In some embodiments MCT1 expression is assessed by determining the level of an MCT1 gene product. In some embodiments an MCT1 gene product comprises an MCT1 RNA, e.g., MCT1 mRNA. In some embodiments an MCT1 gene product comprises an MCT1 polypeptide. In some embodiments the level of an MCT1 gene product is detected in a sample obtained from a tumor. In some embodiments an MCT1 gene product is detected in a lysate or extract prepared from a sample. In some embodiments an MCT1 gene product is detected using a method that allows detection of the gene product in individual cells that express it. In some embodiments detecting an MCT1 gene product comprises contacting a sample with an appropriate detection reagent for such MCT1 gene product and detecting binding of the detection reagent to the gene product by, e.g., detecting the detection reagent bound to the gene product.

In general, any suitable method for measuring RNA can be used to measure the level of an MCT1 RNA, e.g., MCT1 mRNA, in a sample. For example, methods based at least in part on hybridization and/or amplification can be used. The sample may comprise RNA that has been isolated from a cell or tissue sample or RNA may be detected within cells. Exemplary methods of use to detect mRNA include, e.g., in situ hybridization, Northern blots, microarray hybridization (e.g., using cDNA or oligonucleotide microarrays), reverse transcription PCR, nanostring technology (see, e.g., Geiss, G., et al., Nature Biotechnology (2008), 26, 317-325; U.S. Ser. No. 09/898,743 (U.S. Pat. Pub. No. 20030013091) for exemplary discussion of nanostring technology and general description of probes of use in nanostring technology). It will be understood that mRNA may be isolated and/or reverse transcribed to cDNA, which may be further copied, e.g., amplified, prior to detection. In some embodiments detecting MCT1 mRNA comprises reverse transcription of mRNA, followed by PCR amplification with primers specific for a MCT1 mRNA. Thus it will be understood that in various embodiments detection of mRNA may comprise detecting mRNA molecules and/or detecting a DNA or RNA copy or reverse copy thereof. In some embodiments real-time PCR (also termed quantitative PCR), e.g., reverse transcription real-time PCR is used. Commonly used real time PCR assays include the TaqMan® assay and the SYBR® Green PCR assay. In some embodiments multiplex PCR is used, e.g., to quantify MCT1 mRNA and at least one additional mRNA. It will be understood that certain methods of use to detect mRNA may, in at least some instances, also detect at least some pre-mRNA transcript(s), transcript processing intermediates, and degradation products of sufficient size. In some embodiments a method designed to specifically detect mRNA is used. For example, a polyT primer may be used to reverse transcribe mRNA, which may then be selectively amplified and/or detected.

In some embodiments the level of a target nucleic acid is determined by a method comprising contacting a sample with one or more nucleic acid probe(s) and/or primer(s) comprising a sequence that is substantially or perfectly complementary to the target nucleic acid over at least 10, 12, 15, 20, or 25 nucleotides, maintaining the sample under conditions suitable for hybridization of the probe or primer to its target nucleic acid, and detecting or amplifying a nucleic acid that hybridized to the probe or primer. In some embodiments, “substantially complementary” refers to at least 90% complementarity, e.g., at least 95%, 96%, 97%, 98%, or 99% complementarity. In some embodiments the sequence of a probe or primer is sufficiently long and sufficiently complementary to MCT1 mRNA (or its complement) to allow the probe or primer to distinguish between MCT1 mRNA (or its complement) and at least 95%, 96%, 97%, 98%, 99%, or 100% of transcripts (or their complements) from other genes in a mammalian cell, e.g., a human cell, under the conditions of an assay. In some embodiments, a probe or primer may also comprise sequences that are not complementary to a MCT1 mRNA (or its complement). In some embodiments such additional sequences do not significantly hybridize to other nucleic acids in a sample and/or do not interfere with hybridization to MCT1 mRNA (or its complement) under conditions of the assay. In some embodiments, an additional sequence may be used, for example, to immobilize a probe or primer to a support or to serve as an identifier or “bar code”. In some embodiments, a probe or primer hybridizes to a target nucleic acid in solution. The probe or primer may subsequently immobilized to a support. In some embodiments a probe or primer is attached to a support prior to hybridization to a target nucleic acid. Methods for attaching probes or primers to a support will be apparent to those of ordinary skill in the art. For example, oligonucleotide probes can be synthesized in situ on a surface or nucleic acids (e.g., cDNAs, PCR products) can be spotted or printed on a surface using, e.g., an array of fine pins or needles often controlled by a robotic arm that is dipped into wells containing the probes and then used to deposit each probe at a designated location on the surface.

In some embodiments a probe or primer is labeled. A probe or primer may be labeled with any of a variety of detectable labels. In some embodiments a label is a radiolabel, fluorescent small molecule (fluorophore), quencher, chromophore, or hapten. Nucleic acid probes or primers may be labeled during synthesis or after synthesis. In some embodiments a nucleic acid to be detected (e.g., MCT1 mRNA or cDNA) is labeled prior to detection, e.g., prior to or after hybridization to a probe. For example, in microarray-based detection, nucleic acids in a sample may be labeled prior to being contacted with a microarray or after hybridization to the microarray and removal of unhybridized nucleic acids. Methods for labeling nucleic acids and performing hybridization and detection will be apparent to those of ordinary skill in the art. Microarrays are available from various commercial suppliers such as Affymetrix, Inc. (Santa Clara, Calif., USA) and Agilent Technologies, Inc. (Santa Clara, Calif., USA). For example, GeneChips® (Affymetrix) may be used, such as the GeneChip® Human Genome U133 Plus 2.0 Array or successors thereof. Microarrays may comprise one or more probes or probe sets designed to detect each of thousands of different RNAs. In some embodiments a microarray comprises probes designed to detect transcripts from at least 2,500, at least 5,000, at least 10,000, at least 15,000, or at least 20,000 different genes, e.g., human genes.

In some embodiments MCT1 RNA level is measured using a sequencing-based approach such as serial analysis of gene expression (SAGE) (including modified versions thereof) or RNA-Sequencing (RNA-Seq). RNA-Seq refers to the use of any of a variety of high throughput sequencing techniques to quantify RNA molecules (see, e.g., Wang, Z., et al. Nature Reviews Genetics (2009), 10, 57-63). Other methods of use for detecting RNA include, e.g., electrochemical detection, bioluminescence-based methods, fluorescence-correlation spectroscopy, etc.

In some embodiments increased copy number of a chromosomal region containing the MCT1 gene or at least a portion of the MCT1 gene sufficient to encode a functionally active MCT1 polypeptide serves as an indicator of increased MCT1 expression. In some embodiments copy number of a region is considered increased in a population of cells (e.g., tumor cells in a sample) if more than 2 copies of the region are present in at least 10%, 20%, 30%, 40%, 50%, 60%, 70%, 80%, 90%, 95%, or more of the cells analyzed. In some embodiments copy number in at least 10%, 20%, 30%, 40%, 50%, 60%, 70%, 80%, 90%, 95%, or more of the cells analyzed may be at least 3, 4, 5, 8, 10, or 15. In some embodiments copy number of a region is considered increased in a population of cells if the average copy number per cell is greater than 2.0. In some embodiments, average copy number may be at least 3, 4, 5, 8, 10, or 15. Methods useful for assessing copy number include, e.g., fluorescence in situ hybridization (FISH), multiplex ligation-dependent probe amplification, quantitative multiplex PCR of short fluorescent fragments (QMPSF), comparative genomic hybridization, array comparative genomic hybridization, SNP array technologies, DNA sequencing, etc. Copy number alteration (CNA) information is available in various databases. For example, CaSNP is a database containing CNA information inferred from cancer SNP array data (Cao. Y., et al., Nucl. Acids Res. (2010) doi: 10.1093/nar/gkq997 First published online: Oct. 23, 2010). It is available at http://cistrome.dfci.harvard.edu/CaSNP/. Tumorscape (available at http://www.broadinstitute.org/tumorscape/pages/portalHome.jsf) contains copy number data amassed from multiple cancer types (Beroukhim et al, Nature, 463:899-905, 2010)

In some embodiments an MCT1 gene product comprises an MCT1 polypeptide. In general, any suitable method for measuring proteins can be used to measure the level of MCT1 polypeptide in a sample. Numerous strategics that may be used for detection of a polypeptide are known in the art. Exemplary detection methods include, e.g., immunohistochemistry; immunofluorescence, enzyme-linked immunosorbent assay (ELISA), bead-based assays such as the Luminex® assays (Life Technologies/Invitrogen, Carlsbad, Calif.), flow cytometry, protein microarrays, surface plasmon resonance assays (e.g., using BiaCore technology), microcantilevers, immunoprecipitation, immunoblot (Western blot), etc. In some embodiments an immunological method or other affinity-based method is used. In general, immunological detection methods involve detecting specific antibody-antigen interactions in a sample such as a tissue section or cell sample. The sample is contacted with an antibody that binds to the target antigen of interest. The antibody is then detected using any of a variety of techniques. In some embodiments, the antibody that binds to the antigen (primary antibody) or an antibody (secondary antibody) that binds to the primary antibody has a detectable label attached thereto. In general, assays may be performed in any suitable vessel or on any suitable surface. In some embodiments multiwell plates are used.

In some embodiments, MCT1 protein is detected using an ELISA assay. Traditional ELISA assays typically involve use of primary or secondary antibodies that are linked to an enzyme, which acts on a substrate to produce a detectable signal (e.g., production of a colored product) to indicate the presence of antigen or other analyte. As used herein, the term “ELISA” also encompasses use of non-enzymatic reporter molecules such as fluorogenic, electrochemiluminescent, or real-time PCR reporter molecules that generate quantifiable signals. It will be appreciated that the term “ELISA” encompasses a number of variations such as “indirect”, “sandwich”, “competitive”, and “reverse” ELISA. Examples of various assays and devices suitable for performing immunoassays or other affinity-based assays are described in U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; 5,480,792; 4,727,022; 4,659,678; and/or 4,376,110.

In some embodiments MCT1 protein is detected using immunohistochemistry (IHC). IHC generally refers to the immunological detection of an antigen of interest (e.g., a cellular or tissue constituent) in a tissue or cell sample comprising substantially intact cells, which may be fixed and/or permeabilized. As used herein, IHC encompasses immunocytochemistry (ICC), which term generally refers to the immunological detection of a cellular constituent in isolated cells that essentially lack extracellular matrix components and tissue microarchitecture that would typically be present in a tissue sample. In some embodiments, e.g., where IHC is used for detecting MCT1, a sample is in the form of a tissue section, which may be a fixed or a fresh (e.g., fresh frozen) tissue section or cell smear in various embodiments. In some embodiments fixation of cells may, for example, be performed by exposing them to 1% paraformaldehyde for 10 minutes at 37 degrees C., which may be followed by permeabilization, e.g., in 90% methanol for about 30 minutes on ice. In some embodiments a sample, e.g., a tissue section, may be embedded, e.g., in paraffin or a synthetic resin or combination thereof. A sample may be fixed using a suitable fixative such as a formalin-based fixative. In some embodiments a tissue section is a paraffin-embedded, formalin-fixed tissue section. A tissue section may be deparaffinized—a process in which paraffin (or other substance in which the tissue section has been embedded) is removed at least sufficiently to allow staining of a portion of the tissue section. To facilitate the immunological reaction of antibodies with antigens in fixed tissue or cells it may be helpful to unmask or “retrieve” the antigens through pretreatment of the sample. A variety of procedures for antigen retrieval (sometimes called antigen recovery) can be used. Such methods can include, for example, applying heat (optionally with pressure) and/or treating with various proteolytic enzymes. Methods can include microwave oven irradiation, combined microwave oven irradiation and proteolytic enzyme digestion, pressure cooker heating, autoclave heating, water bath heating, steamer heating, high temperature incubator, etc. To reduce background staining in IHC, the sample may be incubated with a buffer that blocks the reactive sites to which the primary or secondary antibodies may otherwise bind. Common blocking buffers include, e.g., normal serum, non-fat dry milk, bovine serum albumin (BSA), or gelatin, and various other available blocking buffers. The sample is then contacted with an antibody that specifically binds to the antigen whose detection is desired (e.g., MCT1 protein). After an appropriate period of time, unbound antibody is removed (e.g., by washing), and antibody that remains bound to the sample is detected. After immunohistochemical staining, a second stain may be applied, e.g., to provide contrast that helps the primary stain stand out. Such a stain may be referred to as a “counterstain”. Such stains may show specificity for discrete cellular compartments or antigens or may stain the whole cell. Examples of commonly used counterstains include, e.g., hematoxylin, Hoechst stain, or DAPI. A tissue section can be visualized using appropriate microscopy, e.g., light microscopy, fluorescence microscopy, etc.

Protein microarrays are arrays that comprise a plurality of capture reagents, e.g., detection reagents such as antibodies, immobilized on a support. The array is contacted with a sample under conditions suitable for analytes in the sample to bind to the capture reagents. Unbound material may be removed by washing. Analytes that bound to a capture reagent are detected using any of a variety of approaches. In some embodiments the array is contacted with a second reagent, such as a second detection reagent capable of binding to an analyte of interest. See, e.g., U.S. Patent Pub. Nos. 20030153013 and 20040038428 for examples of protein microarrays and methods of making and using them.

In some embodiments, flow cytometry (optionally including cell sorting) is used to detect MCT1 expression. Flow cytometry is typically performed on isolated cells suspended in a liquid. For example, a tissue sample may be processed to isolate cells from surrounding tissue. The cells are contacted with a detection reagent that binds to MCT1 mRNA (e.g., a nucleic acid probe) or that binds to MCT1 protein (e.g., an antibody), washed to remove unbound detection reagent, and subjected to flow cytometry. The detection reagent is appropriately labeled (e.g., with a fluorescent moiety) so as to be detectable by flow cytometry.

In some embodiments an antibody used in an immunological detection method is monoclonal. In some embodiments an antibody is polyclonal. In some embodiments, an antibody preparation comprises multiple monoclonal antibodies, which may bind to the same epitope or different epitopes of MCT1. Antibodies can be generated using full length MCT1 as an immunogen or binding target or using one or more fragments of MCT1 as an immunogen or binding target. In some embodiments, an antibody is an anti-peptide antibody. Antibodies capable of detecting MCT1 protein, e.g., human MCT1 protein, are commercially available. For example, monoclonal antibody Ab3540 (Millipore, Inc., Billerica, Mass.) may be used. One of ordinary skill in the art would be able, using standard methods such as hybridoma technology or phage display, to generate additional antibodies suitable for use to detect MCT1 polypeptide.

In some embodiments, a ligand that specifically binds to MCT1 polypeptide and that is not an antibody is used as a detection reagent. For example, nucleic acid aptamers or various non-naturally occurring polypeptides that are structurally distinct from antibodies may be used. Examples include, e.g., agents referred to in the art as affibodies, anticalins, adnectins, synbodies, etc. See, e.g., Gebauer, M. and Skerra, A., Current Opinion in Chemical Biology, (2009), 13(3): 245-255 PCT/DE1998/002898(published as WO/1999/016873), or PCT/US2009/041570 (published as WO/2009/140039). Such agents may be used to detect MCT1 protein in a similar manner to antibodies.

In some embodiments an antibody or other binding agent, e.g., a detection reagent, binds to MCT1 polypeptide with a K_{d}, of 10⁻⁴ or less, e.g., 10⁻⁵ M or less, e.g., 10⁻⁶ M or less, 10⁻⁷ M or less, 10⁻⁸ M or less, 10⁻⁹ M or less, or 10⁻¹⁰ M or less.

In some embodiments, a non-affinity based method such as mass spectrometry may be used to assess the level of MCT1 polypeptide.

In some embodiments MCT1 expression may be detected in a tumor in vivo by administering an appropriate detection reagent to a subject. In some embodiments the detection reagent binds to an MCT1 gene product, e.g., MCT1 protein, and is then detected by, for example, a suitable detector or imaging method. The amount of detection reagent bound to the tumor provides an indication of the amount of MCT1 gene product expressed. Useful molecular imaging modalities include molecular MRI (mMRI), magnetic resonance spectroscopy, optical bioluminescence imaging, optical fluorescence imaging, ultrasound, single-photon emission computed tomography (SPECT), positron emission tomography (PET), and combinations thereof. The detection reagent may comprise a label to render it more readily detectable. A label may be a radionuclide such as ¹²³I, ¹¹¹In, ^{99m}Tc, ⁶⁴Cu, or ⁸⁹Zr; a fluorescent moiety, magnetic or paramagnetic particle, microbubble (for ultrasound-based detection), quantum dot (semiconductor nanoparticles), nanocluster, etc. In some embodiments the detection reagent is detected noninvasively. In some embodiments the detection reagent may be detected at the time of surgery to remove a tumor or using a probe or endoscope, which may be equipped with a detector.

A reagent, e.g., detection reagent such as an antibody that binds to MCT1 polypeptide or a probe or primer that hybridizes to MCT1 mRNA or to a complement thereof, or a procedure for use to detect an MCT1 gene product may be validated, if desired, by showing that a classification or prediction obtained using such detection reagent or procedure on an appropriate set of test samples correlates with a characteristic of interest such as sensitivity to 3-BrPA or likelihood of therapeutic response to 3-BrPA. For example, in some embodiments, an antibody or a procedure for performing IHC may be validated by establishing that its use provides similar results to those obtained using antibody Ab3540 on an appropriate set of test samples. In some embodiments a detection reagent or procedure may be validated by establishing that its use results in the same classification of at least 80%, 85%, 90%, 95%, 96%, 97%, 98%, 99% or more of samples as obtained using the antibody Ab3540, e.g., as described in the Examples. A set of test samples may be selected to include, e.g., at least 3, 5, 10, 20, 30, or more samples in each category in a classification system (e.g., high MCT1 expression, low MCT1 expression). In some embodiments, a set of test samples comprises samples from tumors of a particular tumor type or tissue of origin. Once a particular reagent or procedure has been validated it can be used to validate additional reagents or procedures.

Suitable controls, normalization procedures, or other types of data processing can be used to accurately quantify MCT1 expression, where appropriate. In some embodiments measured values are normalized based on total mRNA or total protein or total cell number in a sample. In some embodiments measured values are normalized based on the expression of one or more RNAs or polypeptides whose expression is not correlated with a characteristic of interest such as sensitivity to 3-BrPA and the expression level of which is not expected to vary greatly between tumor cells and non-tumor cells or is not expected to vary greatly among tumors in general or is not expected to vary greatly among tumors of the tumor type to which a particular tumor belongs. In some embodiments the gene used for normalization encodes a ribosomal protein, e.g., ribosomal protein S6.

In some embodiments a measured value for the level of an MCT1 gene product is normalized to account for the fact that different samples may contain different proportions of a cell type of interest, e.g., cancer cells versus non-cancer cells (e.g., stromal cells). Cells may be distinguished by their expression of various cellular markers. For example, in some embodiments the percentage of stromal cells, e.g., fibroblasts, may be assessed by measuring expression of a stromal cell-specific marker, and the result of a measurement of MCT1 RNA or polypeptide level in the sample may be adjusted to accurately reflect MCT1 RNA or polypeptide level specifically in the tumor cells. It will be understood that if a sample contains distinguishable areas of neoplastic and non-neoplastic tissue (e.g., based on standard histopathological criteria), such as at the margin of a tumor, the level of MCT1 expression may be assessed specifically in the area of neoplastic tissue, e.g., for purposes of classifying the tumor according to predicted sensitivity to 3-BrPA or an analog thereof or other purposes described herein. In some embodiments a level measured in non-neoplastic tissue of the sample may be used as a reference level for purposes of comparison, e.g., as described herein.

In some embodiments a background level, which may reflect non-specific binding of a detection reagent, may be subtracted from a measured value of MCT1 gene product level.

In some embodiments multiple measurements are performed on a tumor sample and/or or multiple tumor samples from a tumor are assessed. In some embodiments the number of measurements performed on a sample or the number of samples assessed is between 2 and 10. In some embodiments an average value of MCT1 expression level is used.

In some embodiments the level of a gene product, e.g., an MCT1 gene product, is determined to be “increased” or “not increased” or “high” or “low” as compared with a reference level. A reference level may be a predetermined value, or range of values (e.g. from analysis of a set of samples) determined to correlate with increased sensitivity to 3-BrPA or increased likelihood of sensitivity to 3-BrPA. Any method herein that includes a step of assessing the level of MCT1 gene expression may comprise a step of comparing the level of MCT1 gene expression with a reference level of MCT1 gene expression, wherein if the measured level is determined to be greater than the reference level, then the measured level is considered to be “increased” (or, if the measured level is not greater than the reference level, then the measured level is considered to be “not increased”). For example, in some embodiments, if a tumor cell, tumor cell line, or tumor has an increased level of MCT1 expression as compared to a reference level, the tumor cell, tumor cell line, or tumor is classified as having an increased likelihood of being sensitive to 3-BrPA or an analog thereof, while if the tumor cell, tumor cell line, or tumor does not have a an increased level of MCT1 relative to a reference level, the tumor is classified as having a decreased likelihood of being sensitive to 3-BrPA or an analog thereof. In some embodiments a reference level is an absolute level. In some embodiments a reference level is a relative level, such as a proportion of cells that exhibit strong staining for MCT1 protein.

In some embodiments comparing an MCT1 gene product level with a reference level may comprise determining a difference between the measured level and the reference level, e.g., by subtracting the reference level from the measured level or may comprise determining a ratio. For example, in some embodiments an “increased” level of an MCT1 gene product refers to a level of MCT1 gene product at least about 1.1, 1.2, 1.5, 2, 2.5, 3, 5, 10, 20, 50, 100, 250, 500, 1000-fold, or more, greater than a reference level. A comparison may involve subjecting the results of one or more measurements to any appropriate statistical analysis.

In some embodiments a reference level of an MCT1 gene product is a level or range of levels found in one or more tumors or tumor cell lines that is sensitive to or resistant to 3-BrPA. In a case in which a reference level is characteristic of a tumor or tumor cell line that is sensitive to 3-BrPA, the presence of an MCT1 gene product in a tumor cell, tumor cell line, sample, or tumor at a level comparable to, e.g., approximately the same, as or greater than the reference level would, for example, be predictive of sensitivity to 3-BrPA or an analog thereof or would identify a subject who is a candidate for treatment with 3-BrPA or an analog thereof, while a decreased level an MCT1 gene product as compared with the reference level would indicate less likelihood of sensitivity to 3-BrPA (e.g., greater likelihood of resistance) or would identify a subject who may not be a candidate for treatment with 3-BrPA or an analog thereof. In some embodiments a reference level of an MCT1 gene product is a level or range of levels found in one or more tumors or tumor cell lines that is not sensitive to 3-BrPA. In a case in which a reference level is characteristic of a tumor or tumor cell line that is that is not sensitive to 3-BrPA, the presence of an MCT1 gene product in a sample or tumor at a level comparable to, e.g., approximately the same, as or less than the reference level would, for example, be predictive of lack of sensitivity (resistance) to 3-BrPA or an analog thereof, or would identify a subject who is not a candidate for treatment with 3-BrPA or an analog thereof, while an increased level of MCT1 gene product as compared with the reference level would, for example, be predictive of increased likelihood of sensitivity to 3-BrPA or an analog thereof or would identify a subject who may be a candidate for treatment with 3-BrPA or an analog thereof.

In some embodiments MCT1 expression data obtained from a panel of tumor reference samples are used to establish reference level(s) that represent increased or decreased MCT1 expression or to establish reference level(s) that represent high, intermediate, or low MCT1 expression levels. In some embodiments the reference samples are from cancers that are known to be sensitive or resistant to 3-BrPA. In some embodiments reference levels of MCT1 expression that correlate, with 3-BrPA sensitivity or resistance with at least a specified correlation coefficient (e.g., at least 80%, at least 90%, or more) are established. In some embodiments, a method may comprise determining a reference level. Reference samples may be of a particular tumor type, e.g., liver, breast, lung, pancreatic, kidney, etc., or a particular subtype, such as triple negative breast tumors. In some embodiments a reference level is a level that has been determined using the same type of sample, comparable handling of the sample, same type of MCT1 gene product (e.g., mRNA or protein), and same or equivalent detection technique as for the subject or tumor being tested.

In some embodiments archived tissue samples, which may be in the form of one or more tissue microarrays (TMA), are used. Tissue microarrays may be produced by obtaining small portions (e.g., disks) of tissue from various types of standard histologic sections (e.g., formalin-fixed paraffin-embedded (FFPE) samples) or from newly obtained samples and placing or embedding them in a regular arrangement (e.g., in mutually perpendicular rows and columns) on or in a substrate such as a paraffin block. A tissue microarray may comprise many, e.g., dozens or hundreds of samples (e.g., between about 50 and about 1000 samples), which can be analyzed in parallel and using uniform analysis conditions. See, e.g., Kononen J, et al., Tissue microarrays for high-throughput molecular profiling of tumor specimens. Nat Med 1998, 4:844-847; Equiluz, C., et al., Pathol Res Pract., 202(8):561-8, 2006. TMAs may be prepared using a hollow needle to remove tissue cores (e.g., as small as about 0.6 mm in diameter) from paraffin-embedded tissue samples. These tissue cores are then inserted in a paraffin block in an array pattern. Sections from such a block can be cut, e.g., using a microtome, mounted on a microscope slide, and then analyzed by any method of analysis, e.g., standard histological analysis methods such as IHC or FISH. Each microarray block can be cut into 100-500 sections, which can be subjected to independent tests.

In some embodiments cancers falling within the upper quartile of MCT1 expression (i.e., the 25% of tumors having the highest MCT1 expression level) are classified as having increased likelihood of sensitivity to 3-BrPA as compared with cancers falling within the lowest three quartiles (i.e., the 75% of tumors having the lowest MCT1 expression). In some embodiments cancers falling within the upper tercile of MCT1 expression (i.e., the 33% of tumors having the highest MCT1 expression level) are classified as having increased likelihood of sensitivity to 3-BrPA as compared with cancers falling within the lowest two terciles (i.e., the 66% of tumors having the lowest MCT1 expression levels). In some embodiments cancers falling within the upper tercile of MCT1 expression (i.e., the 33% of tumors having the highest MCT1 expression level) are classified as having increased likelihood of sensitivity to 3-BrPA as compared with cancers falling within the lowest tercile (i.e., the 33% of tumors having the lowest MCT1 expression levels). In some embodiments cancers falling at or above the median level of MCT1 expression (i.e., the 50% of tumors having the highest MCT1 expression level) are classified as having increased likelihood of sensitivity to 3-BrPA as compared with cancers falling below the median (i.e., the 50% of tumors having the lowest MCT1 expression level). In some embodiments the tumors are of a particular type or tissue of origin. The levels of MCT1 expression that correlate with 3-BrPA sensitivity or resistance in tumors of a particular type or tissue of origin may be used for classifying other tumors, e.g., other tumors of that type or tissue of origin. In some embodiments levels of MCT1 expression that correlates with a specified correlation coefficient (e.g., at least 0.80, at least 0.85, at least 0.90, at least 0.925, at least 0.95, or more) with 3-BrPA sensitivity or resistance in tumors as a whole or tumor of a particular type or tissue of origin are used. In some embodiments a correlation coefficient is a Pearson correlation coefficient. In some embodiments a correlation coefficient is a Spearman correlation coefficient. In some embodiments a correlation between MCT1 expression and sensitivity to 3-BrPA reflects a linear or approximately linear relationship or monotonic relationship between MCT1 expression level and the effect of 3-BrPA. For example, a tumor cell or tumor that has a high level of MCT1 expression will tend to be more sensitive to being killed or its proliferation or growth inhibited by 3-BrPA than a tumor cell or tumor that has a lower level of MCT1 expression. In some embodiments, an effect of an agent, e.g., 3-BrPA, may be expressed as the 50% inhibitory concentration (IC₅₀), defined as the lowest concentration of agent that results in a 50% decrease in the parameter being assessed (e.g., enzyme activity, cell number, cell survival, cell proliferation, glycolytic activity) as compared with a control in which the agent is absent or essentially absent (e.g., undetectable). If desired, an IC₉₀ can be assessed in a similar manner. Sensitivity may be expressed in terms of IC₅₀ or IC₉₀, where a lower IC₅₀ or IC₉₀ indicates a higher degree of sensitivity.

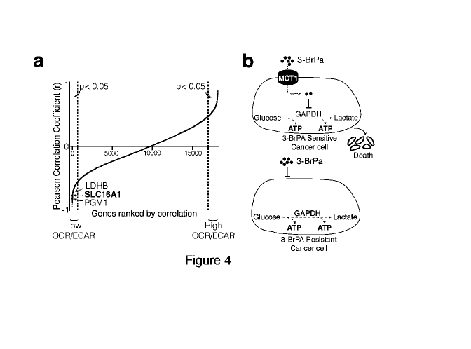

In some embodiments a reference level is a level that represents a normal level of MCT1 gene product, e.g., a level of MCT1 gene product existing in non-cancer cells or tissue, e.g., normal, healthy cells or tissue. For example, in some embodiments MCT1 expression is considered to be increased in a tumor cell, tumor cell line, or tumor if the level of MCT1 expression is at least 4 times as high as that found in normal cells of the same cell type or tissue of origin, e.g., at least 5, 6, 8, 12, 16, 32, 50, 100, 1000-fold, or more, as high as that exhibited by normal cells of the same tissue of origin. In some embodiments a sample comprises both tumor tissue and non-tumor tissue. In some embodiments one or more samples are obtained from a tumor, and one or more samples are obtained from nearby, e.g., adjacent, normal (non-tumor) tissue composed of similar cell types from the same patient. The relative level of MCT1 gene product in the tumor tissue versus the non-tumor tissue and/or in the tumor sample(s) versus the non-tumor sample(s) is determined and used to assess whether MCT1 expression is increased in the tumor. Examples of levels of MCT1 expression in normal tissue of a variety of tissue types are provided herein (see Supplemental FIG. 4B).

A measured value or reference level may be semi-quantitative, qualitative, or approximate. For example, visual inspection (e.g., using microscopy) of a stained IHC sample can provide an assessment of the level of MCT1 expression without necessarily counting cells or precisely quantifying the intensity of staining. In some embodiments one or more steps of a method described herein is performed at least in part by a machine, e.g., computer (e.g., is computer-assisted) or other apparatus (device) or by a system comprising one or more computers or devices. In some embodiments a computer is used in sample tracking, data acquisition, and/or data management. For example, in some embodiments a sample ID is entered into a database stored on a computer-readable medium in association with a measurement of MCT1 expression. The sample ID may subsequently be used to retrieve a result of determining MCT1 expression in the sample. In some embodiments, automated image analysis of a sample is performed using appropriate software, comprising computer-readable instructions to be executed by a computer processor. For example, a program such as ImageJ (Rasband, W. S., ImageJ, U.S. National Institutes of Health, Bethesda, Md., USA, http://imagej.nih.gov/ij/, 1997-2012; Schneider, C. A., et al., Nature Methods 9: 671-675, 2012; Abramoff, M. D., et al., Biophotonics International, 11(7): 36-42, 2004) or others having similar functionality may be used. In some embodiments, an automated imaging system is used. In some embodiments an automated image analysis system comprises a digital slide scanner. In some embodiments the scanner acquires an image of a slide (e.g., following IHC for detection of MCT1) and, optionally, stores or transmits data representing the image. Data may be transmitted to a suitable display device, e.g., a computer monitor or other screen. In some embodiments an image or data representing an image is added to a patient medical record.

In some embodiments, whether or not MCT1 expression is considered “increased” in a cell, cell line, or sample, may be determined by comparing the level of expression of MCT1 with the level of expression of at least some other genes. In some embodiments MCT1 expression is considered increased if MCT1 is among the 30, 40, or 50 most highly expressed genes, among a set of genes that includes MCT1 wherein the set includes at least 10,000 or at least 12,000 or at least 15,000 or substantially all human genes.

In some embodiments a machine, e.g., an apparatus or system, is adapted, designed, or programmed to perform an assay for measuring expression of MCT1. In some embodiments an apparatus or system may include one or more instruments (e.g., a PCR machine), an automated cell or tissue staining apparatus, a device that produces, records, or stores images, and/or one or more computer processors. The apparatus or system may perform a process using parameters that have been selected for detection and/or quantification of an MCT1 gene product, e.g., in tumor samples. The apparatus or system may be adapted to perform the assay on multiple samples in parallel and/or may comprise appropriate software to provide an interpretation of the result. The apparatus or system may comprise appropriate input and output devices, e.g., a keyboard, display, printer, etc. In some embodiments a slide scanning device such as those available from Aperio Technologies (Vista, Calif.), e.g., the ScanScope AT, ScanScope CS, or ScanScope FL or is used.

In some embodiments an assessment of MCT1 expression is used as a diagnostic test, which may be referred to as a “companion diagnostic”, to determine, e.g., whether a patient is a candidate for treatment with 3-BrPA or an analog thereof. In some embodiments a reagent or kit for performing such a diagnostic test may be packaged or otherwise supplied with 3-BrPA or an analog thereof. In some embodiments 3-BrPA or an analog thereof or pharmaceutical composition comprising 3-BrPA or an analog thereof may be approved by a government regulatory agency (such as the US FDA or government agencies having similar authority over the approval of therapeutic agents in other jurisdictions), e.g., allowed to be marketed, promoted, distributed, sold or otherwise provided commercially for treatment of humans or for veterinary purposes, with a recommendation or requirement that the subject is determined to be a candidate for treatment with 3-BrPA or an analog thereof based at least in part on assessing the level of MCT1 expression in a tumor of the subject to be treated. For example, the approval may be for an indication that includes a requirement that a tumor to be treated has increased levels of MCT1 expression. Such a requirement or recommendation may be included in a package insert or label provided with the 3-BrPA or analog thereof. In some embodiments a particular method for detection or measurement of an MCT1 gene product or a specific detection reagent or specific kit comprising such reagent may be specified.

It will be understood that various methods that are described herein in terms of conclusions or predictions that can be made if increased MCT1 expression is present could be stated in terms of conclusions or predictions that can be made if increased MCT1 expression is not present, e.g., if MCT1 expression is low or absent, and vice versa. For example, if MCT1 expression is absent or low in a tumor sample, the tumor would not be classified as likely to be sensitive to 3-BrPA or an analog thereof. In some embodiments, if MCT1 expression is absent or low in a tumor sample, the subject from whom the sample was obtained would not be a candidate for treatment with 3-BrPA or an analog thereof. In some embodiments, if MCT1 expression is absent or low, the subject is predicted to be unlikely to benefit from treatment with 3-BrPA or an analog thereof. In some embodiments, if MCT1 expression is absent or low, a treatment other than 3-BrPA or an analog thereof is selected.

In certain embodiments any of the methods may comprise assigning a score to a sample (or to a tumor from which a sample was obtained) based at least in part on the level of MCT1 expression measured in the sample. In some embodiments, a score is assigned using a scale of 0 to X, where 0 indicates that the sample is “negative” for MCT1 (e.g., no to minimal detectable MCT1 polypeptide, and X is a number that represents strong (high intensity) staining of the majority of cells. In some embodiments, a score is assigned using a scale of 0, 1, or 2, where 0 indicates that the sample is negative for MCT1 (e.g., no or minimal detectable MCT1 polypeptide), 1 is low to moderate level staining and 2 is strong (intense) staining of the majority of tumor cells. It will be understood that staining need not be evident throughout the cell. For example, staining may be strongest at the cell membrane. A higher score indicates a higher likelihood of sensitivity to 3-BrPA or an analog thereof. In some embodiments X is 2, 3, 4, or 5 in various embodiments. In some embodiments “no detectable MCT1” or “negative for MCT1” means that the level detected, if any, is not noticeably or not significantly different to a background level.

In some embodiments a score is assigned based on assessing both the level of MCT1 expression and the percentage of cells that exhibit increased MCT expression. For example, a score can be assigned based on the percentage of cells that exhibit increased MCT1 expression and the extent to which expression level is increased. For example, a first score (e.g., between 0 and 5) can be assigned based on the percentage of cells that exhibit at least moderate staining for MCT1 and a second score (e.g., between 0 and 5) assigned based on the percentage of cells that exhibit intense staining. In some embodiments, the two scores are combined (e.g., added or multiplied) to obtain a composite score. In some embodiments, the two scores are added or multiplied to obtain a composite score. In some embodiments a range is divided into multiple (e.g., 2 to 5) subranges, and samples or tumors are assigned an overall MCT1 expression score based on the subrange into which the composite score falls. A higher score indicates, for example, increased likelihood of sensitivity to 3-BrPA or an analog thereof. It will be understood that if a tissue sample comprises areas of neoplastic tissue and areas of non-neoplastic tissue a score can be assigned based on expression in the neoplastic tissue. In some embodiments the non-neoplastic tissue may be used as a reference.

In some embodiments at least about 50%, 60%, 70%, 80%, 90%, or more tumor cells assessed express increased levels of MCT1. In some embodiments less than about 50% the cells assessed express increased levels of MCT1, e.g., between about 5% and about 25% or between about 25% and about 50%. In some embodiments, if a tumor comprises at least some cells that express increased MCT1 and at least some cells that do not express increased MCT1, the tumor is treated with 3-BrPA. 3-BrPA may be useful to eliminate the subpopulation of cells that express increased MCT1. In some embodiments the tumor is treated with 3-BrPA in combination with a second anti-tumor therapy.

A score can be obtained by evaluating one field or multiple fields in a cell or tissue sample. In some embodiments multiple samples from a tumor are evaluated. It will be appreciated that a score can be represented using numbers or using any suitable set of symbols or words instead of, or in combination with numbers. For example, scores can be represented as 0, 1, 2; negative, positive; negative, low, high; −, +, ++, +++; 1+, 2+, 3+, etc. In some embodiments, at least 10, 20, 50, 100, 200, 300, 400, 500, 1000 cells, or more, are assessed to evaluate MCT1 expression in a sample or tumor and/or to assign a score to a sample or tumor. In some embodiments the number of cells is up to about 10⁴, 10⁵, 10⁶, 10⁷, 10⁸, or more. The number of cells may be selected as appropriate for the particular assay used and/or so as to achieve a particular degree of accuracy, repeatability, or reproducibility.

In some embodiments, the level of MCT1 expression is used in selecting a dose of 3-BrPA or an analog thereof. For example, a higher dose of the compound or a shorter dosing interval (time between consecutive doses) may be selected if a tumor has an intermediate level of MCT1 expression than if a tumor has a high level of MCT1 expression.

In some embodiments the number of categories in a useful scoring or classification system is least 2, e.g., 2, 3, or 4, or between 4 and 10, although the number of categories may be greater than 10 in some embodiments. In some embodiments a scoring or classification system is effective to divide a population of tumors or subjects into groups that differ in terms of a result or outcome such as response to a treatment (e.g., 3-BrPA) or survival. A result or outcome may be assessed at a given time or over a given time period, e.g., 3 months, 6 months, 1 year, 2 years, 5 years, 10 years, 15 years, or 20 years from a relevant date such as the date of diagnosis or approximate date of diagnosis (e.g., within about 1 month of diagnosis) or a date after diagnosis, e.g., a date of initiating treatment. Various categories may be defined. For example, tumors may be classified as having low, intermediate, or high likelihood of sensitivity or resistance to 3-BrPA or a subject may be determined to have a low, intermediate, or high likelihood of experiencing a clinical response to 3-BrPA. A variety of statistical methods may be used to correlate the likelihood of a particular outcome (e.g., sensitivity, resistance, response, lack of response, survival for at least a specified time period) with the relative or absolute level of MCT1 expression. One of ordinary skill in the art will be able to select and perform appropriate statistical tests. Correlations may be calculated by standard methods, such as a chi-squared test, e.g., Pearson's chi-squared test. Such methods are well known in the art (see, e.g., Daniel, W. W., et al., Biostatistics: A Foundation for Analysis in the Health Sciences, 8th ed. (Wiley Series in Probability and Statistics), 2004 and/or Zar, J., Biostatistical Analysis, 5^{th} ed., Prentice Hall; 2009). Statistical analysis may be performed using appropriate software. Numerous computer programs suitable for performing statistical analysis are available. Examples, include, e.g., SAS, Stata, GraphPad Prism, and many others. R is a programming language and software environment useful for statistical computing and graphics that provides a wide variety of statistical and graphical techniques, including linear and nonlinear modeling, classical statistical tests, classification, clustering, and others.

One of ordinary skill in the art will appreciate that the terms “predicting”, “predicting the likelihood”, and like terms, as used herein, do not imply or require the ability to predict with 100% accuracy and do not imply or require the ability to provide a numerical value for a likelihood. Instead, such terms typically refer to forecast of an increased or a decreased probability that a result, outcome, event, etc., of interest (e.g., sensitivity of a tumor cell or tumor to 3-BrPA or an analog thereof) exists or will occur, e.g., when particular criteria or conditions exist, as compared with the probability that such result, outcome, or event, etc., exists or will occur when such criteria or conditions are not met. In some embodiments a numerical value may be provided, such as an absolute or relative likelihood. In some embodiments an increased likelihood is increased by at least 25%, 50%, 75%, 100%, 200% (2-fold), 300% (3-fold), 400% (4-fold), 500% (5-fold), or more. In some embodiments an increased likelihood is a likelihood of at least 20%, 30%, 40%, 50%, 60%, 70%, 80%, 90%, or more. It will also be understood that a method for predicting the likelihood of tumor cell or tumor sensitivity (or resistance) may comprise or be used together with one or more other methods. For example, assessment of MCT1 expression may be used together with assessment of one or more additional genes, gene products, metabolites, or parameters. In some embodiments one or more such additional measurements may be combined with assessment of MCT1 expression to increase the predictive value of the analysis (e.g., to provide a more conclusive determination of likelihood of sensitivity) in comparison to that obtained from measurement of an MCT1 gene product alone. Thus a method of predicting likelihood can be a method useful to assist in predicting likelihood in combination with one or more other methods. The various components of a set of measurements may be assigned the same or similar weights or may be weighted differently.

In some embodiments, a level of an MCT1 gene product (e.g., mRNA or polypeptide) is assessed and used together with levels of gene product(s) of one or more additional genes, e.g., for classifying a tumor cell, tumor cell line, or tumor according to predicted sensitivity to 3-BrPA or an analog thereof. It will be understood that methods described herein of assessing MCT1 expression, determining whether MCT1 expression is increased or decreased, determining reference levels, etc., may be applied to assess expression of any gene of interest using appropriate detection reagents for gene products of such genes.

In some embodiments expression of a second gene is assessed, wherein the second gene encodes a gene product that promotes, e.g., is necessary for, MCT1 expression or function. For example, in some embodiments the level of BSG mRNA or protein is assessed. In some embodiments a tumor cell, tumor cell line, or tumor that has increased expression of MCT1 is classified as having an increased likelihood of being sensitive to 3-BrPA or an analog thereof if the tumor cell, tumor cell line, or tumor expresses BSG than if the tumor cell, tumor cell line, or tumor does not express BSG. In some embodiments a tumor cell, tumor cell line, or tumor that has increased expression of MCT1 is classified as having an increased likelihood of being sensitive to 3-BrPA or an analog thereof if the tumor cell, tumor cell line, or tumor has increased expression of BSG than if the tumor cell, tumor cell line, or tumor does not have increased expression of BSG. In some embodiments any one or more BSG transcripts or isoforms is detected. In some embodiments a detection reagent, e.g., a probe or antibody, capable of detecting a particular BSG transcript variant or isoform is used. In some embodiments a detection reagent, e.g., a probe or antibody, capable of detecting all of the BSG transcript variants or isoforms is used.

In some embodiments at least one of the additional mRNAs or proteins to be detected is selected based at least in part on its utility for classification for diagnostic, prognostic, or predictive purposes in one or more types of cancer. For example, in the case of breast cancer, MCT1 mRNA or polypeptide levels can be used together with a measurement of estrogen receptor (ER), progesterone receptor (PR), or human epidermal growth factor receptor 2 (HER2) mRNA or polypeptide levels, as discussed further below.

In certain embodiments the level of MCT1 mRNA or protein level is not assessed simply as a contributor to a cluster analysis, dendrogram, or heatmap based on gene expression profiling in which expression at least 10; 20; 50; 100; 500; 1,000, or more genes is assessed. In certain embodiments, if MCT1 mRNA or protein level is measured as part of such a gene expression profile, the level of MCT1 mRNA or protein is used in a manner that is distinct from the manner in which the expression of many or most other genes in the gene expression profile are used. For example, the level of MCT1 mRNA or polypeptide may be used independently of, e.g., without regard to, expression levels of most or all of the other genes or may be weighted more strongly than most or all other levels in analyzing or using the results.

In general, methods disclosed herein may be applied to any tumor cell, tumor cell line, tumor or sample comprising tumor cells. Various tumor types and tumor cell lines are mentioned herein. For example, in some embodiments a tumor is a solid tumor. In some embodiments a solid tumor is a liver, breast, gastrointestinal tract (e.g., colon cancer, esophageal cancer), cervical, ovarian, pancreatic, renal, prostate, esophageal, lung, or brain cancer (e.g., glioblastoma). In some embodiments, a tumor has detectably metastasized when assessed or treated. In some embodiments, a tumor has not detectably metastasized when assessed or treated. In some embodiments, a tumor is a recurrent tumor (i.e., a tumor that reappears after becoming undetectable) or a relapsed tumor (i.e., a tumor that has initially responded to therapy but then worsens). In some embodiments the tumor is resistant to one or more standard chemotherapy agents or regimens.

In some embodiments a tumor is a liver tumor. Hepatocellular carcinoma (HCC, also called malignant hepatoma) is the most common type of primary liver cancer. Most cases of HCC result at least in part from viral hepatitis infection (hepatitis B or C) or hepatic cirrhosis. Other types of liver cancer include cholangiocarcinoma (bile duct cancer), hepatoblastoma (a rare malignant tumor, primarily occurring in children), and angiosarcoma (malignant cancer of endothelial cells that line the walls of blood vessels (hemangiosarcoma) or lymphatic vessels (lymphangiosarcoma)). The liver is also a common site of secondary tumors arising as a result of metastatic spread from a primary tumor of, e.g., the gastrointestinal tract (e.g., colon cancer), pancreas, breast, ovary, lung, kidney, prostate, or melanoma (e.g., ocular melanoma).

In some embodiments a tumor is a breast tumor. In some embodiments a breast tumor can be classified into a particular recognized class or category. For example, breast cancers can be classified into molecular subtypes based on gene expression profiles, e.g., luminal A, luminal B, ERBB2-associated, basal-like, and normal-like (see, e.g., Sørlie, T., et al., Proc Natl Acad Sci USA. (2001) 98(19):10869-74). Breast cancers can be classified based on a number of different clinicopathologic features such as histologic subtype (e.g., ductal; lobular; mixed), histologic grade (grade 1, 2, 3); estrogen receptor (ER), progesterone receptor (PR), and/or HER2 (ERBB2, human epidermal growth factor receptor2) expression status, and lymph node involvement. In some embodiments ER, PR, and/or HER2 expression status (e.g., positive or negative) is determined using IHC. In some embodiments HER2 status is determined by assessing amplification of the HER2 gene, e.g., using in situ hybridization, e.g., FISH. In some embodiments a breast tumor is ER+. In some embodiments a tumor is ER−. In some embodiments a breast tumor is HER2+. In some embodiments a tumor is HER2−. In some embodiments a breast tumor is PR+. In some embodiments a tumor is PR−. In some embodiments a breast tumor is (1) ER+, HER2+; (2) ER+, HER2−; (3) ER−, HER2+; and (4) ER−, HER2−. These subtypes can be further divided based on expression of PR. In some embodiments a breast tumor is EGFR+. In some embodiments a breast tumor is EGFR−. It will be understood that these markers may be present or absent in any combination in various embodiments. In some embodiments, a tumor is “triple negative”, i.e., the tumor is negative for or has minimal expression of ER, PR, and HER2. In some embodiments triple-negative breast cancer is defined by its lack of (or minimal) ER and PR expression, together with the absence of HER2 overexpression or gene amplification. Triple negative breast cancer is frequently an aggressive form of breast cancer, often characterized by early relapse and/or poor response to chemotherapeutic agents. In some embodiments a breast tumor is ductal carcinoma in situ (DCIS). In some embodiments testing for ER, PR, and/or HER2 is performed in accordance with recommendations of the American Society of Clinical Oncology/College of American Pathologists Guideline Recommendations for Immunohistochemical Testing of Estrogen and Progesterone Receptors in Breast Cancer or the American Society of Clinical Oncology/College of American Pathologists Guideline Recommendations for Human Epidermal Growth Factor Receptor 2 Testing in Breast Cancer. In some embodiments such testing is performed according to recommendations of a commercially available kit, e.g., a kit approved by a governmental regulatory agency (e.g., the U.S. Food and Drug Administration) for use in clinical diagnostic, prognostic, or predictive purposes.

In some embodiments MCT1 expression is assessed at a testing facility. A testing facility or individual may be qualified or accredited (e.g., by a national or international organization such as a government organization or a professional organization) to perform an assessment of MCT1 expression, e.g., for purposes of tumor classification for treatment selection purposes. In some embodiments a testing facility is part of or affiliated with a health care facility. In some embodiments a testing facility is not part of or affiliated with a health care facility. It is contemplated that in some embodiments an assay of MCT1 expression may be performed at a testing facility that is remote from (e.g., at least 1 kilometer away from) the site where the sample is obtained from a subject. The testing facility may receive samples from multiple different health care providers. “Health care provider” refers to an individual (e.g., a physician or other health care worker) or an institution (e.g., a hospital, clinic, medical practice, or other health care facility) that provides health care services to individuals on a systematic or regular basis. MCT1 expression may be assessed as part of a panel of molecular pathology tests performed for purposes of tumor classification, diagnosis, prognosis, or treatment selection.

In some embodiments a health care provider seeking to obtain an assessment of MCT1 expression provides a sample (e.g., a tumor sample) to a testing facility with instructions to assess MCT1 expression. In some embodiments providing a sample to a testing facility encompasses directly providing the sample (e.g., sending or transporting the sample), arranging for or directing or authorizing another individual or entity to send or transport, etc. Thus in some embodiments an assessment of MCT1 expression is obtained by a requestor, e.g., a health care provider, by requesting that such assessment be performed, e.g., by a testing facility. The term “requesting” in this context encompasses instructing, urging, demanding, directing, ordering, inducing, persuading, prompting, overseeing, arranging for, or otherwise causing another individual or entity to perform a method or step. In some embodiments a first individual or entity assists a second individual or entity in performing a step or method by, for example, providing: a sample, information about a sample, a detection reagent suitable for performing a step or method, a kit or detection device adapted to perform a step or method, or instructions for performing a method. The first individual or entity may or may not request that the method or step be performed. “Request” in this context is used interchangeably with “order”, “command”, “direct”, and like terms.

In some embodiments a sample is provided to a testing facility within no more than 1, 2, 3, 5, 7, 10, 14, 21, or 28 days after having been removed from a subject. The testing facility measures MCT1 expression in the sample and provides a result. In some embodiments obtaining an assessment of MCT1 expression comprises entering an order for an assay of such expression into an electronic ordering system, e.g., of a health care facility. In some embodiments obtaining an assessment of MCT1 expression comprises receiving a result of measuring MCT1 expression from a testing facility. In some embodiments obtaining an assessment of MCT1 expression comprises retrieving the result of an assessment of MCT1 expression from a database. In some embodiments a method of performing a diagnostic test comprises: (a) receiving a tumor sample obtained from a subject in need of treatment for a tumor; and (b) assessing expression of MCT1 in the tumor sample. In some embodiments the method comprises receiving a request to assess expression of MCT1 in the tumor sample or receiving a request to provide a result of assessing MCT1 expression in the tumor sample. In some embodiments the method further comprises providing a result of an assessment of MCT1 expression to a person or entity that provided the sample or made the request, such as a subject's health care provider. In some embodiments the result is provided by the testing facility within no more than 1, 2, 3, 5, 7, 10, 14, 21, or 28 days after having received the sample.

A result may be provided in any suitable format and/or using any suitable means. In some embodiments a result is provided in an electronic format; optionally a paper copy is provided instead of or in addition to an electronic format. In some embodiments a result is provided at least in part by entering the result into a computer, e.g., into a database, electronic medical record, laboratory information system (sometimes termed laboratory information management system), etc., wherein it may be accessed by or under direction of a requestor. In some embodiments a result may be provided via phone, voicemail, fax, text message, or email. In some embodiments a result is provided at least in part over a network, e.g., the Internet. In some embodiments a result comprises one or more numbers or scores representing an expression level and/or a narrative description. In some embodiments a result includes a classification of a tumor according to predicted sensitivity to 3-BrPA or an analog thereof. In some embodiments a result indicates whether or not a tumor expresses sufficient MCT1 such that a subject in need of treatment for the tumor is a candidate for treatment with 3-BrPA or an analog thereof. In some embodiments a result of assessing MCT1 expression is provided together with additional information regarding a tumor or sample. Additional information may comprise, e.g., assessment of tumor grade, tumor stage, tumor type (e.g., cell type or tissue of origin) and/or results of assessing expression of one or more additional genes. In some embodiments a result is provided in a report.

In some embodiments a requestor (e.g., health care provider) treats a subject or selects a treatment for a subject based at least in part on the results of the assessment. In some embodiments the result indicates that the tumor has increased expression of MCT1, and the treatment used or selected is 3-BrPA or an analog thereof.

In some aspects, the invention provides methods of modulating (altering, e.g., increasing or decreasing) the sensitivity of a cell to 3-BrPA or an analog thereof. In some embodiments a method of modulating sensitivity of a cell to 3-BrPA or an analog thereof comprises modulating the level or activity of MCT1 expressed by the cell. In some embodiments the method comprises increasing the level or activity of MCT1, thereby increasing sensitivity of the cell to 3-BrPA or an analog thereof. In some embodiments the level or activity of MCT1 is increased by introducing an expression construct or expression vector encoding MCT1 into the cell. In some embodiments the method comprises decreasing the level or activity of MCT1 in the cell, thereby increasing sensitivity of the cell to 3-BrPA or an analog thereof. In some embodiments the level or activity of MCT1 is inhibited by contacting the cell with an MCT1 inhibitor. Examples of MCT1 inhibitors are described further below.

In some embodiments, disclosed herein are 3-BrPA analogs. Where the present disclosure refers to 3-bromopyruvate, it should be understood that aspects and embodiments in which an analog, prodrug, salt, or metabolite of 3-bromopyruvate is used instead of or in addition to 3-bromopyruvate are also disclosed. Thus with regard to each aspect or embodiment herein that refers to 3-bromopyruvate, aspects or embodiments are provided in which an analog, prodrug, salt, or metabolite of 3-bromopyruvate is used instead of or in addition to 3-bromopyruvate.

In some embodiments, a 3-BrPA analog is represented by the following formula:

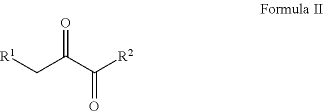

wherein R¹ represents halogen, —S(O)₃R³, —C(O)₂R³, —OR³, or —N+(R³)₂O⁻, wherein each occurrence of R³ independently represents H, CX₃, CHX₂, CH₂X, C1-C12 aliphatic, C1-C12 heteroaliphatic, aryl, or heteroaryl, wherein X represents halogen; R² represents —O⁻, —OR, —H, —N(R⁴)₂, C1-C12 aliphatic, C1-C12 heteroaliphatic, aryl, or heteroaryl, wherein R represents H, alkali metal, C1-C12 aliphatic, C1-C12 heteroaliphatic, aryl, heteroaryl, or C(O)R⁵; and R⁵ represents H, C1-C12 aliphatic, C1-C12 heteroaliphatic, aryl, or heteroaryl; and each occurrence of R⁴ independently represents H, C1-C12 aliphatic, C1-C12 heteroaliphatic, aryl, or heteroaryl. In certain embodiments R³ represents —CH₃C₆H₄, —CH₃, —CF₃, or —C₆H₅.

In some embodiments R¹ represents halogen, —S(O)₃R³, —C(O)₂R³, —OR³, —N⁺(R³)₂O⁻; R² represents O⁻, —OR, or −OH, and R and R³ are as set forth above.

In some embodiments R¹ represents halogen; R² represents O⁻, —OR, or —OH; and R is as set forth above. In some embodiments R represents an aliphatic or heteroaliphatic group (cyclic, acyclic, substituted, unsubstituted, branched or unbranched), e.g., an alkyl group, having 1-6 carbon atoms.

In certain embodiments R¹ represents halogen, R² represents OR, H, N(R⁴)₂, C1-C6 alkyl, C6-C12 aryl, C1-C6 heteroalkyl, or a C6-C12 heteroaryl; each R⁴ independently represents H, C1-C6 alkyl, or C6-C12 aryl; R represents H, alkali metal, C1-C6 alkyl, C6-C12 aryl or C(O)R⁵; and R⁵ represents H, C1-C20 alkyl or C6-C12 aryl.

In some embodiments R¹ represents halogen, R² represents —OR; and R represents an aliphatic or heteroaliphatic group (cyclic, acyclic, substituted, unsubstituted, branched or unbranched), e.g., an alkyl group, having 1-6 carbon atoms.

The term “hetero” indicates that a compound includes one or more heteroatoms. Heteroatom means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)). The terms “halo” and “halogen” as used refer to an atom selected from the group consisting of fluorine, chlorine, bromine, and iodine. In certain embodiments a halogen is bromine (Br).

Definitions of additional terms and examples of chemical moieties and groups, e.g., protecting groups, may be found in International Patent Application PCT/US2009/005656 (published as WO2010044885).

In some embodiments, any of the compounds disclosed herein is isotopically enriched at one or more positions of the compound. For example, compounds having the present structures in which a hydrogen is replaced by deuterium or tritium, or in which a carbon atom (C) is a ¹³C- or ¹⁴C atom, or a fluorine atom (F) is ¹⁸F, or an iodine atom (I) is a ¹²³I, ¹²⁴I, ¹²⁵I, or ¹³¹I are within the scope of this disclosure. In various embodiments such compounds are useful, for example, as analytical tools, as probes in biological assays, or as therapeutic agents.

In some embodiments a 3-BrPA analog has increased stability in aqueous solution as compared with 3-BrPA. In some embodiments a 3-BrPA analog has increased stability in blood, plasma, or serum as compared with 3-BrPA. In some embodiments a 3-BrPA analog has increased potency for inhibiting survival and/or proliferation of tumor cells that have elevated MCT1 expression as compared with 3-BrPA. In some embodiments a 3-BrPA analog has increased uptake by MCT1 as compared with 3-BrPA.

### III. Exploiting Transporter-mediated Transport to Deliver Toxic Agents to Tumors

Transporters are membrane proteins involved in the movement of substances such as ions or small molecules across biological membranes. Transporters may be involved in moving a substance across a cellular membrane that it otherwise would typically not cross, either because it is one to which the phospholipid bilayer of the membrane is impermeable or because it is moved in the direction of the concentration gradient (i.e., into a cell that already contains a higher concentration of the substance than present outside the cell). Transporters may assist in the movement of substances by facilitated diffusion or active transport. Active transport is the movement of a substance across a cell membrane against its concentration gradient, i.e., from lower to higher concentration. A cellular membrane may be the plasma membrane or an intracellular membrane such as an inner or outer mitochondrial membrane, a nuclear membrane, or any membrane enclosing a cellular compartment the interior of which is a site of action for a toxic agent.

In some aspects, the present disclosure encompasses the recognition that transporters expressed by tumor cells can be exploited to selectively deliver toxic agents to such cells or to a cellular compartment within such cells. In some aspects, the disclosure provides methods of identifying agents that enter cancer cells via a transporter that is expressed, e.g., at an elevated level, by at least some tumor cells. In some embodiments, increased expression of a transporter by tumor cells, as compared with normal cells, results in selective uptake of a toxic agent by tumor cells and thereby results in selective inhibition of survival and/or proliferation of such cells. Toxic agents that are taken up into cells by particular transporter(s) may be of particular use to treat tumors that express increased levels of such transporter(s). An agent, the effect of which on cells is at least in part dependent on expression of a particular transporter by the cells may be referred to as a “transporter-dependent” agent. A toxic agent, the toxicity of which depends at least in part on expression of a particular transporter, may be referred to as a transporter-dependent toxic agent. The expression level of a transporter can be assessed and used as a biomarker for sensitivity to toxic agents taken up by the transporter, e.g., to identify tumors or tumor cells that have an increased likelihood of being sensitive to such agents. For example, the present disclosure provides the recognition that MCT1-mediated transport can be exploited to deliver toxic agents to tumor cells that express MCT1 in order to inhibit survival and/or proliferation of such cells. Toxic agents that are taken up by MCT1 may be of particular use to treat tumors that express increased levels of MCT1, as described herein in regard to 3-BrPA. MCT1 expression can be used as a biomarker for sensitivity to such agents to identify tumors that have an increased likelihood of being sensitive to such agents.

Numerous transporters are known. For example, the solute carrier (SLC) group of membrane transport proteins include over 300 members organized (in humans) into at 51 families (Hediger M A, et al. (2004) Pflugers Arch 447 (5): 465-8, and other articles in the same volume; Hoglund P J, et al., Mol Biol Evol. (2011) 28(4):1531-41). Information regarding human SLC family members (SLC1-SLC51 family members), including accession numbers, tissue distribution, and predominant substrates transported thereby, may be found at http://www.bioparadigms.org/slc/menu.asp. Other transporters include, e.g., members of the ATP-binding cassette (ABC) and ATPase membrane protein superfamilies. The Transporter Classification Database (TCDB) provides a comprehensive IUBMB approved classification system for membrane transport proteins known as the Transporter Classification (TC) system. Descriptions, TC numbers, and examples of over 600 families of transport proteins are provided at http://www.tcdb.org/ (Saier M H Jr, et al. Nucl. Acids Res., 37: D274-8; Saier M H Jr, et al., Nucl. Acids Res., 34: D181-6).

In some embodiments a transporter of interest herein is a protein that normally mediates transport of one or more substances into mammalian cells, i.e., across the plasma membrane. Certain of these transporters also mediate movement of one or more substances out of the cell. In some embodiments a transporter mediates movement of a nutrient (e.g., a sugar or amino acid), cofactor, metabolic substrate, or precursor across the cell membrane and into the cell. In some embodiments the nutrient, cofactor, metabolic substrate or precursor may be utilized at higher levels by at least some tumor cells than by non-tumor cells of the same cell type or tissue of origin. Increased expression of the transporter may help support such increased utilization. In some instances a tumor cell or tumor may be particularly dependent on a biological pathway, process, protein, or other cellular component that utilizes a substance taken up via the transporter. In some embodiments the transporter transports a metabolite, metabolic byproduct, or degradation product out of cells. The metabolite, byproduct, or degradation product may be one that would become toxic if present at excessive levels inside the cell. In some embodiments the metabolite, byproduct, or degradation product may be produced at higher levels by at least some tumor cells than by non-tumor cells of the same cell type or tissue of origin. Increased expression of the transporter may help support extrusion of the metabolite, byproduct, or degradation product and thereby promote tumor cell survival.

In some embodiments a transporter of interest herein is a protein that normally mediates transport of one or more substances from the cytoplasm into a membrane-bound cellular compartment such as a mitochondrion or the nucleus. Such transporters may be exploited to deliver toxic agents into such compartment, wherein the toxic agent is one that acts inside the compartment. For example, a toxic agent that inhibits DNA or RNA synthesis or other processes that take place in the nucleus would be suitable for delivery via a transporter that mediates entry of one or more substances into the nucleus.

In some aspects, the present disclosure provides methods of identifying a transporter useful for delivery of toxic agents to tumor cells. In some embodiments a method of identifying a transporter useful for delivery of a toxic agent to tumor cells comprises identifying a transporter that is differentially expressed in at least some tumor cell lines or tumors as compared with normal cells and/or is differentially expressed among tumor cell lines or tumors. Such transporters may be identified, for example, by assessing expression or copy number of genes encoding transporters in multiple tumor cell lines or tumor samples, or by examining publicly available previously measured gene expression data or copy number data, which may be found in various databases as described herein (e.g., Gene Expression Omnibus, Oncomine, etc.) Expression or copy number may be assessed using any suitable method (see, e.g., Section II).

Tumor cells, tumor cell lines, or tumors that express an increased level of a transporter of interest can be identified as described herein with regard to MCT1. In some embodiments an increased level of a transporter refers to a level that is increased as compared with normal tissue or cells, e.g., of the same tissue of origin or cell type as the tumor cells, tumor cell line, or tumors. In some embodiments an increased level of a transporter refers to a level that is increased as compared with at least some other tumor cell lines or tumors. In some embodiments expression of a gene that encodes a transporter is assessed in a set of tumor samples by measuring the level of RNA encoding the transporter or measuring the level of the transporter. In some embodiments a gene that encodes a transporter is characterized in that its expression level varies by a factor of at least 1.5, 2, 3, 5, 10, 20, 30, 40, 50, 75, 100-fold, or more, among a set of tumors, tumor samples, or tumor cell lines, i.e., the highest expression level is at least 1.5, 2, 3, 5, 10, 20, 30, 40, 50, 75, 100-fold as high as the lowest expression level among tumors, tumor samples, or tumor cell lines, or the highest expression level is at least 1.5, 2, 3, 5, 10, 20, 30, 40, 50, 75, 100-fold as high as the level in non-cancer cells or tissues of the same type or tissue of origin. In some embodiments for example, at least 0.1%, 0.5%, 1%, 2.5%, 5%, 10% or more, of tumors or tumor cell lines analyzed express the transporter at a level at least 5 times as great as the level at which it is expressed by non-cancer cells or tissues of the same type or tissue of origin. In some embodiments a set of samples includes samples from at least 5, 10, 20, 30, 40, 50, 60, 70, 80, 90, 100, 150, 200, 250, 300, 400, 500, or more tumors, e.g., human tumors. In some embodiments a transporter comprises multiple polypeptide chains (“subunits”). In some instances at least some of the subunits are encoded by distinct genes. Expression of one or more of such genes may be measured to assess the level of the transporter. In some embodiments expression is assessed using a method that detects transporters located in the membrane where they are active.

In some embodiments a genetic screen using near-haploid mammalian cells is used to identify a transporter that promotes sensitivity of cell to a toxic agent. In some embodiments the transporter is characterized in that functional inactivation of the transporter confers resistance to a toxic agent. In some embodiments the transporter is useful to deliver a toxic agent to tumor cells. In some embodiments a genetic screen using near-haploid mammalian cells is used to identify a toxic agent, the toxicity of which is at least partly dependent on the expression level of a particular transporter. In some embodiments, sensitivity of the cell to the toxic agent correlates with expression level of the transporter. The expression level of such transporter(s) may be used as a biomarker for sensitivity of tumor cells to the toxic agent. In some embodiments the transporter is expressed at significantly higher levels in at least some tumors than in non-tumor cells of the same cell type or tissue or origin. Tumors may be classified according to the level of expression of the transporter. Such classification may be used to identify tumors that have an increased likelihood of responding to treatment with a toxic agent that is taken up via the transporter.

Methods of performing genetic screens in near-haploid mammalian cells are described in PCT/US2010/041628 (published as WO/2011/006145); Carette, J E, et al., (2009); Science 326, 1231; Carette, J E, et al., (2011); Nat Biotechnol 29, 542; and/or Carette, J E, et al., (2011); Nature 477, 340. Example 1 describes use of a genetic screen in near-haploid cells to identify MCT1 as the transporter responsible for uptake of 3-BrPA by tumor cells. In some embodiments a method of identifying a transporter that promotes sensitivity of a cell to a toxic agent comprises: (a) providing a plurality of mutagenized mammalian cells; (b) contacting the plurality of mutagenized mammalian cells with a toxic agent; (c) isolating a cell that exhibits decreased sensitivity (increased resistance) to the toxic agent; and (d) identifying a gene that is mutagenized in the cell, wherein the gene encodes a transporter, thereby identifying a transporter that promotes sensitivity to the toxic agent. In some embodiments the mutagenized mammalian cells are near-haploid cells. As used herein, a “near-haploid” mammalian cell refers to a mammalian cell in which no more than 5 chromosomes are present in two or more copies. In some embodiments a near-haploid mammalian cell has no more than 1, 2, 3, or 4 chromosomes present in two or more copies. As used herein, the terms “near-haploid” and “haploid” are used interchangeably and encompass fully haploid cells, which contain no more than one copy of each chromosome, and cells that have two or more copies of 1, 2, 3, 4, or 5 chromosomes. For purposes herein, if at least half of the genetic information present on a normal chromosome, as assessed using FISH or by examining banding pattern, remains present within a cell, the chromosome is considered to be present.

In some embodiments the mutagenized mammalian cells are human cells. In some embodiments the mutagenized mammalian cells are KBM7 cells. The term “KBM7 cell line” encompasses near-haploid cell lines isolated from the original KBM7 cell line and subclones therefrom. As will be appreciated, KBM7 subclones can be further subcloned to give rise to additional KBM7 subclones. Similarly, other near-haploid cell lines can be further subcloned. In some embodiments a near-haploid mammalian cell is a leiomyosarcoma cell (Dal Sin, P., et al., J. Pathol., 185(1):112-5, 1988), a malignant fibrous histiocytomas (MFH) cell (Aspberg F, et al., Cancer Genet Cytogenet. 1995; 79(2):119-2.), a breast cancer cell (Flagiello D, Cancer Genet Cytogenet. 1998; 102(1):54-8), a mesothelioma cell or a malignant peripheral nerve sheath tumor cell, or an embryonic stem (ES) cell. Sukov W R, et al., Cancer Genet Cytogenet. 2010; 202(2):123-8 describes certain near-haploid cells of use in certain embodiments.

In some embodiments the mammalian cells are insertionally mutagenized, e.g., by a gene trap vector. In some embodiments step (c) comprises isolating a cell that exhibits increased resistance to the toxic agent as compared to control cells. In some embodiments step (c) comprises isolating a cell that exhibits increased resistance to the toxic agent as compared to control cells, and step (d) comprises identifying the gene as a gene that encodes a transporter. In some embodiments step (a) comprises contacting the plurality of mutagenized mammalian cells with a toxic agent at a concentration and for a time sufficient to kill at least 95% of control cells; step (c) comprises isolating surviving cells; and step (d) comprises identifying a gene that is mutated in at least some of the surviving cells, wherein the gene encodes a transporter, thereby identifying a transporter that is at least in part responsible for toxicity of the toxic agent. In some embodiments the method comprises: (b) contacting the plurality of mutagenized mammalian cells with the toxic agent at a concentration and for a time sufficient to kill at least 95% of control cells, wherein members of the population have decreased functional expression of different genes; (c) isolating cells that survive; and (d) identifying a gene whose mutation frequency in surviving cells is significantly greater than a reference frequency. In some embodiments the reference frequency is approximately equal to (i) the mutation frequency of the gene in the cells of step (a); or (ii) an estimated average mutation frequency of the gene in unselected cells. In some embodiments the toxic agent is a known chemotherapeutic agent.

In some aspects, the present disclosure provides methods of testing an agent for its ability to inhibit the survival and/or proliferation of a tumor cell that expresses a transporter of interest. An agent to be assessed or that is being assessed or has been assessed, e.g., with regard to its effect on cell survival or proliferation or any other parameter of interest, may be referred to as a “test agent”. Any of a wide variety of agents may be used as test agents in various embodiments. For example, a test agent may be a small molecule, polypeptide, peptide, nucleic acid, oligonucleotide, lipid, carbohydrate, or hybrid molecule. Nucleic acids may be RNAi agents, e.g., siRNA or shRNA, or may be antisense oligonucleotides or may be cDNAs or portions thereof or other nucleic acids that can be expressed in cells, optionally encoding proteins. Agents can be obtained from natural sources or produced synthetically. Agents may be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. A library may comprise, e.g., between 100 and 500,000 compounds, or more. In some embodiments compounds are arrayed in multiwell plates. They may be dissolved in a solvent (e.g., DMSO) or provided in dry form, e.g., as a powder or solid. Collections of synthetic, semi-synthetic, and/or naturally occurring compounds may be tested. Compound libraries can comprise structurally related, structurally diverse, or structurally unrelated compounds. Compounds may be artificial (having a structure invented by man and not found in nature) or naturally occurring. In some embodiments a library comprises at least some compounds that have been identified as “hits” or “leads” in a drug discovery program and/or analogs thereof. A compound library may comprise natural products and/or compounds generated using non-directed or directed synthetic organic chemistry. A compound library may be a small molecule library. Other libraries of interest include peptide or peptoid libraries, cDNA libraries, oligonucleotide libraries, and RNAi libraries. A library may be focused (e.g., composed primarily of compounds having the same core structure, derived from the same precursor, or having at least one biochemical activity in common). Compound libraries are available from a number of commercial vendors such as Tocris BioScience, Nanosyn, BioFocus, and from government entities such as the U.S. National Institutes of Health (NIH). In some embodiments, a test agent which is an “approved human drug” may be tested. An “approved human drug” is an agent that has been approved for use in treating humans by a government regulatory agency such as the US Food and Drug Administration, European Medicines Evaluation Agency, or a similar agency responsible for evaluating at least the safety of therapeutic agents prior to allowing them to be marketed. A test agent may be, e.g., an antineoplastic, antibacterial, antiviral, antifungal, antiprotozoal, antiparasitic, antidepressant, antipsychotic, anesthetic, antianginal, antihypertensive, antiarrhythmic, antiinflammatory, analgesic, antithrombotic, antiemetic, immunomodulator, antidiabetic, lipid- or cholesterol-lowering (e.g., statin), anticonvulsant, anticoagulant, antianxiety, hypnotic (sleep-inducing), hormonal, or anti-hormonal drug, etc. Examples of approved drugs are found in, e.g., Goodman and Gilman's The Pharmacological Basis of Therapeutics, and/or Katzung, B., cited above. In some embodiments a test agent is a known anti-cancer agent. In some embodiments a test agent is not a known anti-cancer agent. In some embodiments a test agent is not an agent that is known to be present in detectable amounts in an ordinary cell culture medium, e.g., a cell culture medium ordinarily used for culturing tumor cells. In some embodiments, if a cell culture medium ingredient is used as a test agent, it is used at a concentration at least 5 times higher than that in which it is found in such ordinary cell culture medium.

In some embodiments analogs of a molecule known to be transported by a particular transporter is tested for potential as anti-tumor agents. In some embodiments an analog comprises a toxic moiety. In some embodiments one or more agents to be tested may be designed, e.g., using structural information about the transporter and/or about substances that are transported by it. For example, computational methods may be used. In some embodiments virtual screening is used to identify molecules that may be transported by a transporter of interest. Such molecules may then be tested in physical assays.

In some embodiments a method of testing the ability of an agent to inhibit the survival and/or proliferation of a tumor cell comprises: (a) contacting one or more test cells with an agent, wherein the one or more test cells has increased expression of a transporter as compared to control cells; and (b) assessing the level of inhibition of the survival and/or proliferation of the one or more test cells by the agent. In some embodiments the transporter is characterized in that it is expressed at increased levels by at least some tumors. In some embodiments the method comprises (c) comparing the level of inhibition of the survival and/or proliferation of the one or more test cells by the agent with the level of inhibition of the survival and/or proliferation of control cells by the agent; and (d) identifying the agent as a candidate transporter-dependent modulator of cell survival or proliferation if the level of inhibition of the survival and/or proliferation of the one or more test cells by the agent differs from the level of inhibition of the survival and/or proliferation of the one or more control cells by the agent. In some embodiments the method comprises (c) comparing the level of inhibition of the survival and/or proliferation of the one or more test cells by the agent with the level of inhibition of the survival and/or proliferation of control cells by the agent; and (d) identifying the agent as a candidate transporter-dependent inhibitor of cell survival or proliferation if the level of inhibition of the survival and/or proliferation of the one or more test cells by the agent is greater than the level of inhibition of the survival and/or proliferation of the one or more control cells by the agent. Thus if the agent exhibits greater inhibitory effect on test cells than control cells, the agent may be identified as a candidate transporter-dependent anti-tumor agent. In some embodiments the method comprises contacting one or more control cells with the agent; and assessing the level of inhibition of the survival and/or proliferation of the one or more control cells by the agent. In some embodiments the level of inhibition of the survival and/or proliferation of control cells by the agent is already known or available and the comparison may be performed using such level without contacting one or more control cells with the agent. In some embodiments the one or more test cells and/or one or more control cells are tumor cells.

In some aspects, the present disclosure provides methods of identifying a candidate anti-tumor agent. In some embodiments a method of identifying a candidate anti-tumor agent comprises: (a) contacting one or more test cells with an agent, wherein the one or more test cells has increased expression of a gene that encodes a transporter as compared with expression of the gene by one or more control cells; and (b) assessing the level of inhibition of survival or proliferation of the one or more test cells by the agent. In some embodiments the method further comprises (c) identifying the agent as a candidate anti-tumor agent if the agent has a greater inhibitory effect on survival or proliferation of the one or more test cells than it has on control cells. In some embodiments the method comprises contacting one or more control cells with the agent and assessing the level of inhibition of survival or proliferation of the one or more test cells by the agent. In some embodiments step (c) comprises identifying the agent as a candidate transporter-dependent anti-tumor agent. In some embodiments a result of measuring the effect of the agent on one or more control cells is already known or available, and such measurement is compared with the effect of the agent on the one or more test cells. In some embodiments the one or more test cells and/or one or more control cells are tumor cells.

For purposes of convenience, in describing methods and products (e.g., compositions, culture vessels or other articles) that involve or comprises test cells and control cells that differentially express a transporter, it will be assumed that test cells have increased expression of the transporter relative to control cells. However, it will be appreciated that methods and products could equally well be described using the term “control cells” to refer to cells that have increased expression of a transporter as compared with test cells. If such nomenclature were used, an agent may be identified as a candidate anti-tumor agent if the agent has a greater inhibitory effect on survival or proliferation of the control cell(s) than it has on test cell(s).

In some embodiments test cells and control cells are genetically matched, e.g., in that they originate from a single individual, cell or tissue sample, cell line, or cell, or from genetically identical (isogenic) or essentially genetically identical individuals (e.g., monozygotic twins, animals from an inbred strain), cell or tissue samples, cell lines, or cells. The term “essentially” is used in this context to encompass the possibility that cells may not be genetically identical even if they originate from a single cell, sample, or individual. For example, cells may acquire mutations in culture or in vivo and thus the genomic sequence of two cells derived from a single cell or individual may differ at one or more positions. In some embodiments, test cells and/or control cells are derived from isogenic or essentially isogenic and have undergone no more than 2, 3, 5, 10, 15, 20, 25, 30, 35, 40, 50, 60, 70, 80, 90, or 100 population doublings or passages following isolation as individual cell lines or cell populations before being used in a screen or assay to identify candidate anti-tumor agents.

In some embodiments test cells and/or control cells are modified to cause them to express a transporter at increased or decreased levels. For example, in some embodiments test cells are genetically modified to cause them to express increased levels of the transporter as compared with the control cells and/or control cells are genetically modified to cause them to have reduced expression of the transporter as compared with the test cells. Methods of producing genetically modified cells are well known in the art. For example in some embodiments test cells are generated from an initial cell population by introduction of a vector comprising a sequence that encodes a transporter, so that the resulting cells express increased levels of the transporter as compared with cells that have not been so manipulated. In some embodiments test cells are caused to have reduced expression of a gene that encodes a transporter by contacting them with an RNAi agent. In some embodiments cells are contacted with exogenous siRNA. In some embodiments a vector that comprises a template for transcription of a short hairpin RNA or antisense RNA targeted to a gene or transcript encoding the transporter is introduced into cells, such that the resulting cells express an shRNA or antisense RNA that inhibits expression of the gene. A nucleic acid construct or vector may be introduced into cells by transfection, infection, or other methods known in the art. Cells may be contacted with an appropriate reagent (e.g., a transfection reagent) to promote uptake of a nucleic acid or vector by the cells. In some embodiments a genetic modification is stable such that it is inherited by descendants of the cell into which a vector or nucleic acid construct was introduced. A stable genetic modification usually comprises alteration of a cell's genomic DNA, such as integration of exogenous nucleic acid into the genome or deletion of genomic DNA. A nucleic acid construct or vector may comprise a selectable marker that facilitates identification and/or isolation of genetically modified cells and, if desired, establishment of a stable cell line. It will be understood that the term “genetically modified” refers to an original genetically modified cell or cell population and descendants thereof. Thus a genetically modified cell used in methods described herein may be a descendant of an original genetically modified cell.

In some embodiments test cells are caused to have reduced expression of a gene that encodes a transporter by functionally inactivating the gene by, e.g., inserting a nucleic acid into the gene, or deleting at least a portion of the gene, or otherwise altering the sequence so as to reduce the function of the gene. In some embodiments the function of a gene is substantially decreased so that expression is not detectable or is detectable at insignificant levels. This may be achieved by a variety of mechanisms, including introduction of a disruption of the coding sequence, e.g., insertion of one or more stop codons; insertion into or deletion of at least a portion of a coding or non-coding sequence (e.g., regulatory regions such as the promoter region, 3′ regulatory sequences, enhancers), etc. Insertions or deletions may be random or targeted. Targeted insertions or deletions may be performed using homologous recombination. In some embodiments, a cell may be genetically modified using an endonuclease that is targeted to selected DNA sequences, e.g., within a gene that encodes a transporter. Examples include zinc-finger nucleases (ZFNs) and TALENs. ZFNs comprise DBDs derived from or designed based on DBDs of zinc finger (ZF) proteins. TALENs comprise DBDs derived from or designed based on DBDs of transcription activator-like (TAL) effectors of plant pathogenic Xanthomonas spp. (See, e.g., WO2011097036; Urnov, F D, et al., Nature Reviews Genetics (2010), 11: 636-646; Miller J C, et al., Nat. Biotechnol. (2011) 29(2):143-8; Cermak, T., et al. Nucleic Acids Research (2011) 39 (12): e82, and references in any of the foregoing). Expression may be measured to identify those cells or subclones that have increased or decreased expression, as desired, or to confirm that such increased or decreased expression is maintained.

In some embodiments the level of a transporter or the level of mRNA encoding a transporter differs by at least 1.5, 2, 3, 5, 10, 20, 30, 50, 100-fold or more between test cells and control cells, i.e., test cells have at least 1.5, 2, 3, 5, 10, 20, 30, 50, 100, or more times as high a level of the transporter or mRNA encoding the transporter as do control cells.

In some embodiments test cells or control cells that are not genetically modified to have altered expression of a transporter are modified by introduction of a control vector or control nucleic acid that lacks a nucleic acid to be expressed or that comprises a sequence encoding a gene product that is not expected to affect survival or proliferation of the cells or encoding a shRNA targeted to a gene that is not present in or is not expressed by the cells. For example, in some embodiments a control nucleic acid construct or vector may encode GFP or encode an shRNA targeted to GFP. In some embodiments a control nucleic acid construct or vector may encode an mRNA that encodes the transporter but comprises a stop codon located downstream of and close to the start codon so that the mRNA is not translated. The control vector or control nucleic acid construct may be otherwise identical or substantially identical to the vector or nucleic acid construct used to alter expression of the transporter. The test cells and control cells may be subjected to similar or substantially identical procedures and handled in a similar or substantially identical manner so that differences between the test cells and control cells (e.g., differences in the effect of a test agent on the test cells and control cells) can be attributed to differential expression of the transporter.

As described herein, Applicants identified the transporter MCT1 as the main determinant of 3-BrPa uptake and sensitivity. Additional toxic agents taken up by MCT1 may be identified and used to inhibit survival or proliferation of tumor cells, e.g., as a therapeutic strategy for treating cancer. In some aspects, the disclosure provides methods of testing the ability of an agent to inhibit the survival and/or proliferation of a tumor cell, the method comprising: (a) contacting one or more test cells with an agent, wherein the one or more test cells has increased expression of MCT1 as compared to one or more control cells; and (b) assessing the level of inhibition of the survival and/or proliferation of the one or more test cells by the agent. In some embodiments the method further comprises identifying the agent as a candidate anti-tumor agent if the agent inhibits the survival or proliferation of the test cells. In some embodiments the method comprises comparing the effect of the agent on test cells with the effect of the agent on control cells. In some embodiments the method further comprises identifying the agent as a candidate anti-tumor agent if the agent inhibits the survival or proliferation of the test cells and such inhibition is greater than the effect of the agent on control cells. In some embodiments the method further comprises identifying the agent as a candidate MCT1-dependent anti-tumor agent if the agent inhibits the survival or proliferation of the test cells and such inhibition is greater than the effect of the agent on control cells.

In some aspects, the disclosure provides methods of identifying agents that are transported into cells via MCT1 and that inhibit tumor cell survival or proliferation. In some aspects, such agents are useful as anti-tumor agents. In some embodiments, a method of identifying a candidate anti-tumor agent comprises identifying an agent that selectively inhibits survival or proliferation of one or more test cells that have increased expression of MCT1, as compared with the effect of the agent on survival or proliferation of one or more control cells that have lower or absent expression of MCT1. In some embodiments the test cells and control cells are tumor cells. In some embodiments the test cells and control cells are of the same tumor type. In some embodiments the test cells and control cells are of the same tumor cell line, except that the test cells have been modified or selected to have increased expression of MCT1 as compared with the control cells and/or the control cells have been modified or selected to have decreased expression of MCT1 as compared with the test cells. In some embodiments the agent is not 3-BrPA. In some embodiments the agent is an analog of 3-BrPA. In some embodiments the agent is not an analog of 3-BrPA.

A cell line or cell population selected for use in a method described herein may exhibit high or low expression of the transporter. If a cell line or cell population exhibits increased expression of a transporter as compared with non-cancer cells it may be used as a source of test cells. For example, as described herein, KBM7 cells express substantial levels of MCT1. In some embodiments KBM7 cells are used as test cells in a screen to identify agents that are selectively toxic to cells that express increased MCT1. KBM7 subclone cell lines that have gene trap insertions into the MCT1 gene and therefore lack expression of MCT1 were isolated as described in Example 1 and were designated Clone A and Clone B. In some embodiments cells of a KBM7 subclone line with an insertion in the MCT1 gene are used as control cells. In some embodiments, a cell of a KBM7 subclone line having an insertion in the MCT1 gene, such as KBM7 Clone A or Clone B, is genetically modified to cause it to express MCT1. Such a modified KBM7 subclone cell may be used as a test cell.

BT-549, HCC-70, BT-20, MDA-MB-468 and MT-3 are examples of cell lines that have increased levels of MCT1. In some embodiments BT-549, HCC-70, BT-20, MDA-MB-468 and MT-3 cells are used as test cells. In some embodiments a BT-549, HCC-70, BT-20, MDA-MB-468 or MT-3 subclone cell line that has low or absent MCT1 expression is identified or generated and used as a source of control cells. Such subclones may be generated by causing the cells to express a shRNA targeted to MCT1, e.g., as described in Example 3.

SK-BR-3, MDA-MB-231, MDA-MB-453, and T47D are examples of cell lines that express low levels of MCT1. In some embodiments SK-BR-3, MDA-MB-231, MDA-MB-453, or T47D are used as control cells. In some embodiments SK-BR-3, MDA-MB-231, MDA-MB-453, or T47D subclone cells that express higher levels of MCT1 are identified or generated and used as test cells. Such subclones may be generated by introducing a vector encoding MCT1 into the cells, e.g., as described in Example 3.

In general, cells of any cell line, e.g., any tumor cell line, may serve as test cells or control cells or may be modified to generate test cells or control cells for use in methods described herein. Approaches described herein for producing or identifying test cells and control cells that have different levels of expression of MCT1 may be applied in the context of any transporter to generate pairs of isogenic or substantially isogenic cell populations, e.g., cell lines, for use as test cells and control cells. In some embodiments a cell line arising from a spontaneously arising tumor, e.g., a human tumor, is used. In some embodiments an experimentally produced tumor cell or an immortalized, non-transformed cell is used. In general, the cell line or cell population may be of any cell type or tissue of origin. In some embodiments multiple pairs of test and/or control cell lines or cell populations are generated or identified, e.g., 2, 3, 5, 10, 15, 20 or more such pairs. For example, multiple pairs of test and/or control cell lines or cell populations may be generated or identified from a particular cell line or cell population of interest and/or multiple pairs of test and/or control cell lines or cell populations may be generated or identified from each of two or more different cell lines or cell populations. In some embodiments two or more different cell lines or cell populations from which test cells and control cells are generated or identified are of the same cell type or tissue of origin. For example, genetically matched pairs of test cells and control cell may be generated from each of multiple different breast cancer cell lines or breast tissue samples. In some embodiments the effect of an agent is tested on multiple pairs of test and/or control cell lines or cell populations, e.g., 2, 3, 5, 10, 15, 20 or more such pairs. In some embodiments, demonstrating that an agent differentially inhibits survival or proliferation of multiple different genetically matched pairs of test and control cells (e.g., has a greater inhibitory effect on test cells than control cells across multiple distinct pairs of test and control cells) gives rise to increased confidence that the differential effect is attributable at least in part to differential expression of the transporter, e.g., at least in part to transporter-mediated uptake of the candidate agent. In some embodiments the effect of an agent on multiple cell lines exhibiting different levels of expression of a transporter is determined and the degree of correlation between expression and inhibitory effect is determined. In some embodiments the multiple cell lines are isogenic or substantially isogenic. In some embodiments an agent that exhibits a correlation coefficient of at least 0.8, 0.85, 0.9, or 0.95 is identified.

In some embodiments test cells and control cells are contacted with a test agent in individual vessels (e.g., individual wells of a microwell plate). Survival or proliferation of the test cells and control cells is assessed at one or more time points and the results are compared. In some embodiments, if the test agent has a greater inhibitory effect on the survival or proliferation of test cells than control cells, the test agent is identified as a candidate anti-tumor agent, e.g., for treatment of tumors that have increased expression of the transporter.

In some embodiments a co-culture is used, wherein test cells and control cells, e.g., genetically matched test cells and control cells, are contacted with a test agent in the same vessel (e.g., a well of a microwell plate). Survival or proliferation of the test cells and control cells is assessed at one or more time points and compared. In some embodiments, if the test agent has a greater inhibitory effect on the survival or proliferation of test cells than control cells, the test agent is identified as a candidate anti-tumor agent, e.g., for treatment of tumors that have increased expression of the transporter.

In order to determine the survival or proliferation of test cells and control cells in a co-culture, the test cells and control cells are typically be distinguishable from each other. In some embodiments test cells and control cells are distinguished based on expression level of the transporter. In some embodiments test cells and control cells are distinguishable from each other in one or more ways other than expression level of the transporter. Test cells and control cells may differ with regard to any characteristic that allows the test and control cells to be identified or distinguished from each other. For example, test cells and/or control cells may be modified or labeled in a way that allows them to be distinguished. In some embodiments the modification or labeling is inherited by or transmitted to descendants of the test cells and/or control cells initially present in the co-culture. In some embodiments test cells and control cells are genetically modified to express different gene products or different amounts of a particular gene product. The gene products may be directly or indirectly detectable. For example, test cells and control cells may be modified to express fluorescent proteins that have distinct absorption and/or emission spectra, such that they can be readily distinguished using, e.g., a fluorescence plate reader, FACS analysis, etc. For example, test cells may express GFP (or another protein that emits light in the green region of the spectrum), and control cells may express RFP (or another protein that emits light in the red region of the spectrum). In some embodiments test cells and control cells may express the same detectable protein at different levels, such that they can be distinguished, or either the test cells or control cells may not express the protein.

Test cells and control cells may be present in a co-culture in any proportion. For example, the initial ratio of test cells to control cells may range from 1:99: to 99:1 in various embodiments. In some embodiments, for example, the ratio is 95:5, 90:10, 80:20, 70:30, 60:40, 50:50, 40:60, 30:70, 20:80, 10:90, 5:95. A co-culture may comprise between 1% and 99% test cells, e.g., between 1% and 20% test cells, between 20% and 50%, between 50% and 80%, e.g., about 5%, 10%, 20%, 30%, 40%, 50%, 60%, 70%, 80%, or 90%. In some embodiments the percentage of test cells is between 10% and 90%. In some embodiments the percentage of test cells is between 20% and 80%. In some embodiments the percentage of test cells is between 30% and 70%. In some embodiments the percentage of test cells is between 40% and 60%. The other cells in a co-culture may be control cells, or may include both control cells and additional cells not intended as test cells or control cells. Additional cells may, for example, comprise stromal fibroblasts.

An agent that differentially affects test cells versus control cells may be identified based on the relative number of test cells and control cells in the co-culture during or after a culture period. A culture or co-culture may be monitored continuously during a culture period or at specific time points. In some embodiments vessels in which either test cells or control cells selectively survive or proliferate may be identified without needing to remove cells from the vessel for analysis and/or without detecting individual cells. For example vessels that exhibit an altered fluorescence signal as compared with the signal prior to or in the absence of exposure to an agent being tested may be identified, e.g., using an appropriate detection apparatus such as a fluorescence microscope or plate reader. In some embodiments at least some cells are removed from the vessel and/or individually detected, e.g., to determine the number of test cells and/or control cells. For example, FACS may be used for such purpose.

In some embodiments the methods comprise determining the ratio of test cells to control cells by detecting a distinguishing characteristic of the cells in the co-culture or an aliquot thereof. Such a ratio may be compared with an expected ratio (based on previous measurement) or actual ratio observed in an essentially identical co-culture performed for about the same time in the absence of a test agent to determine whether the test agent differentially affects the test and control cells. If the ratio observed in the co-culture exposed to the test agent deviates significantly from an expected or actual ratio, the test agent is identified as a candidate anti-tumor agent that differentially affects survival or proliferation of cells that express increased levels of the transporter. If the ratio of test to control cells is lower than the expected or actual ratio observed in the absence of a test agent, the test agent is identified as a candidate anti-tumor agent that differentially inhibits survival or proliferation of cells that express increased levels of the transporter. In some embodiments the expected or actual ratio of test cells to control cells during or after culture in the absence of a test agent is the same or about the same as the initial ratio (i.e., the ratio present in the co-culture at the time of initial exposure to the test agent). In such cases, the ratio observed in the co-culture during or after the culture period in the presence of the test agent may be compared with the initial ratio to determine whether the test agent differentially affects the survival or proliferation of the test and control cells. For example, if a co-culture contains an equal number of test and control cells at the time exposure to a test agent begins, and if the test and control cells would normally survive and proliferate at approximately equal rates in the absence of the test agent, then if the co-culture contains 90% control cells and 10% test cells after a specified time period, e.g., 3 days later, the test agent is identified as a candidate anti-tumor agent that differentially inhibits survival or proliferation of cells that express increased levels of the transporter. It will be appreciated that test and control cells may not survive or proliferate at precisely the same rate in the absence of a test agent even under identical standard culture conditions. For example, increased expression of a transporter may alter proliferation of the test cells relative to that of the control cells even in the absence of a test agent. In such instances a comparison may be performed with a ratio that has been adjusted to account for different proliferation rates of the test cells and control cells. One of ordinary skill in the art will be readily able to perform appropriate comparisons and controls to distinguish effects of a test agent from effects due to expression of the transporter per se and thereby effectively identify agents that differentially affect test cells versus control cells. Controls may include, for example, co-cultures of test cells and control cells performed in the absence of a test agent, optionally in the presence of a vehicle such as DMSO in which a test agent may be dissolved when added to the culture vessel.

In various embodiments the number of test agents is at least 10; 100; 1000; 10,000; 100,000; 250,000; 500,000 or more. In some embodiments test agents are tested in individual vessels, e.g., individual wells of a multiwell plate (sometimes referred to as microwell or microtiter plate or dish). In some embodiments a multiwell plate of use in performing an assay or culturing or testing cells or agents has 6, 12, 24, 96, 384, or 1536 wells. Cells can be contacted with one or more test agents for varying periods of time and/or at different concentrations. In certain embodiments cells are contacted with test agent(s) for between 1 hour and 20 days, e.g., for between 12 and 48 hours, between 48 hours and 5 days, e.g., about 3 days, between 5 days and 10 days, between 10 days and 20 days, or any intervening range or particular value. Cells can be contacted with a test agent during all or part of a culture period. Test agents can be added to culture media at the time of replenishing the media and/or between media changes. In some embodiments a compound is tested at 2, 3, 5, or more concentrations. Concentrations may range, for example, between about 10 nm and about 500 μm. For example, concentrations of about 100 nm, 1 μm, 10 μm, 100 μm, and 200 μm may be used.

In some embodiments, a high throughput screen (HTS) is performed. A high throughput screen can utilize cell-free or cell-based assays. High throughput screens often involve testing large numbers of compounds with high efficiency, e.g., in parallel. For example, tens or hundreds of thousands of compounds can be routinely screened in short periods of time, e.g., hours to days. Often such screening is performed in multiwell plates containing, at least 96 wells or other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a FITS of the present invention are described in Macarron R & Hertzberg R P. Design and implementation of high-throughput screening assays. Methods Mol. Biol., 565:1-32, 2009 and/or An WF & Tolliday N J., Introduction: cell-based assays for high-throughput screening. Methods Mol. Biol. 486:1-12, 2009, and/or references in either of these. Useful methods are also disclosed in High Throughput Screening Methods and Protocols (Methods in Molecular Biology) by William P. Janzen (2002) and High-Throughput Screening in Drug Discovery (Methods and Principles in Medicinal Chemistry) (2006) by Jorg Hüser.

The term “hit” generally refers to an agent that achieves an effect of interest in a screen or assay, e.g., an agent that has at least a predetermined level of inhibitory effect on cell survival, proliferation, or other parameter of interest being measured in the screen or assay. Test agents that are identified as hits in a screen may be selected for further testing, development, or modification. In some embodiments a test agent is retested using the same assay or different assays. For example, a candidate anti-tumor agent may be tested against multiple different tumor cell lines or in an in vivo tumor model to determine its effect on tumor cell survival or proliferation, tumor growth, etc. Additional amounts of the test agent may be synthesized or otherwise obtained, if desired. Physical testing or computational approaches can be used to determine or predict one or more physicochemical, pharmacokinetic and/or pharmacodynamic properties of compounds identified in a screen. For example, solubility, absorption, distribution, metabolism, and excretion (ADME) parameters can be experimentally determined or predicted. Such information can be used, e.g., to select hits for further testing, development, or modification. For example, small molecules having characteristics typical of “drug-like” molecules can be selected and/or small molecules having one or more unfavorable characteristics can be avoided or modified to reduce or eliminated such unfavorable characteristic(s).

Additional compounds, e.g., analogs, that have a desired activity can be identified or designed based on compounds identified in a screen. In some embodiments structures of hit compounds are examined to identify a pharmacophore, which can be used to design additional compounds. An additional compound may, for example, have one or more altered, e.g., improved, physicochemical, pharmacokinetic (e.g., absorption, distribution, metabolism and/or excretion) and/or pharmacodynamic properties as compared with an initial hit or may have approximately the same properties but a different structure. For example, a compound may have higher affinity for the molecular target of interest, lower affinity for a nontarget molecule, greater solubility (e.g., increased aqueous solubility), increased stability, increased bioavailability, oral bioavailability, and/or reduced side effect(s), modified onset of therapeutic action and/or duration of effect. An improved property is generally a property that renders a compound more readily usable or more useful for one or more intended uses. Improvement can be accomplished through empirical modification of the hit structure (e.g., synthesizing compounds with related structures and testing them in cell-free or cell-based assays or in non-human animals) and/or using computational approaches. Such modification can make use of established principles of medicinal chemistry to predictably alter one or more properties.

Compositions and articles comprising test cells, control cells, and one or more test agents, e.g., 10, 100, 10³, 10⁴, 10⁵, or more test agents, wherein the cells and test agents are arranged in one or more vessels (e.g., microwell plates) in a manner suitable for assessing effect of the test agents(s) on the cells, are among the aspects of the present invention. Methods of generating isogenic or substantially isogenic test and control cells and cell lines, methods of preparing compositions and articles comprising such cells and, optionally, one or more test agents, are aspects of the invention.

In certain embodiments an agent identified or tested using a method described herein displays selective activity (e.g., inhibition of survival or proliferation, or other manifestation of toxicity) against test cells relative to its activity against control cells. For example, the IC₅₀ and/or IC₉₀ of an agent may be between about 2-fold and about 1000-fold lower, e.g., about 2, 5, 10, 20, 50, 100, 250, 500, or 1000-fold lower, for test cells versus control cells. In some embodiments the IC₅₀ and/or IC₉₀ of an agent may be about 2, 5, 10, 20, 50, 100, 250, 500, or 1000-fold lower for test cells than for non-tumor cells of the same cell type or tissue of origin that have not been manipulated to have increased expression of a transporter that mediates entry of the agent into cells. Greater degrees of selectivity, e.g., between about 1000 and about 10,000-fold, are contemplated in certain embodiments. In some embodiments a dose-response curve is generated for an agent identified in a screen.

Data or results from testing an agent or performing a screen may be stored or electronically transmitted. Such information may be stored on a tangible medium, which may be a computer-readable medium, paper, etc. In some embodiments a method of identifying or testing an agent comprises storing and/or electronically transmitting information indicating that a test agent has one or more propert(ies) of interest or indicating that a test agent is a “hit” in a particular screen, or indicating the particular result achieved using a test agent. A list of hits from a screen may be generated and stored or transmitted. Hits may be ranked or divided into two or more groups based on activity, structural similarity, or other characteristics

Once a candidate anti-tumor agent is identified, e.g., a candidate anti-tumor agent that exhibits transporter-dependent toxicity, additional agents, e.g., analogs, may be generated based on it, and may be tested for anti-tumor effect or other properties. An additional agent, may, for example, have increased uptake via the transporter, increased potency, increased stability, greater solubility, or any improved property. In some embodiments a labeled form of the agent is generated. The labeled agent may be used, e.g., to directly measure transport of the agent via the transporter.

As described herein, Applicants identified the monocarboxylate transporter MCT1 as the main determinant of 3-BrPa uptake and sensitivity. In some aspects, the disclosure provides methods of identifying additional agents that are transported into cells via MCT1 and that inhibit tumor cell survival or proliferation. In some embodiments a method of identifying a candidate anti-cancer agent comprises identifying an agent that selectively inhibits survival or proliferation of one or more test cells that have increased expression of MCT1, as compared with the effect of the agent on survival or proliferation of one or more cells control cells that have lower or absent expression of MCT1. In some embodiments the cells are tumor cells. In some embodiments the test cells and control cells are of the same tumor type. In some embodiments the test cells and control cells are of the same tumor cell line, except that the test cells have been manipulated (e.g., genetically modified) or selected to have increased expression of MCT1 as compared with the control cells and/or the control cells have been manipulated or selected to have decreased expression of MCT1 as compared with the test cells. In some embodiments the agent is not 3-BrPA. In some embodiments the agent is an analog of 3-BrPA. In some embodiments the agent is not an analog of 3-BrPA.

In some embodiments test cells that express an increased level of a transporter of interest and control cells that have low or absent expression of such transporter are used in a screen in which multiple different known anti-tumor agents, e.g., approved chemotherapeutic agents or analogs thereof, are tested for ability to inhibit cell survival or proliferation. Such a screen may identify anti-tumor agents whose efficacy may at least partly depend on expression of particular transporter(s). Agents whose efficacy is at least in part transporter-dependent may be selected for treating tumors that express such transporter and may be avoided for treatment of tumors that lack expression of the transporter.

In some embodiments a method of identifying a transporter that is at least in part responsible for toxicity of a toxic agent comprises: (a) contacting a mammalian test cell with a toxic agent, wherein the test cell has increased or decreased expression of a gene that encodes a transporter as compared to a control cell; (b) determining whether the test cell has altered sensitivity to the toxic agent as compared to the control cell; and (c) identifying the transporter as being at least partly responsible for toxicity of the toxic agent if the test cell has increased expression of the gene and increased sensitivity to the toxic agent as compared to the control cell or if the test cell has decreased expression of the gene that encodes the transporter and exhibits decreased sensitivity to the toxic agent as compared to the control cell. In some embodiments the test cell is a tumor cell.

In some embodiments a panel of paired test and control cell lines is provided, wherein members of each pair differentially express a different transporter. For example, members of a first pair differentially express MCT1; members of a second pair differentially express MCT2; members of a third pair differentially express MCT3; members of a fourth pair differentially express MCT4, etc. The ability of an agent to differentially inhibit survival or proliferation of test cells is determined for each pair. Agents that may be particularly well suited for treating tumors that express increased levels of particular transporters and/or agents that may be poorly suited for treating tumors that lack expression of particular transporters may thereby be identified. Without wishing to be bound by any theory, differential expression of transporters by tumors may at least in part account for variations in patient response to chemotherapeutic agents that exhibit transporter-dependent uptake. In some aspects, methods described herein may be useful to identify such agents and the transporter(s) that at least in part mediate their uptake, and/or to identify tumors for which such agents have increased or decreased likelihood of being effective therapeutic agents.

As noted above, the disclosure provides the insight that expression of a transporter may be used as a biomarker for sensitivity of a cell to toxic agents that are taken up by cells via the transporter. The use of MCT1 expression as a biomarker for sensitivity of tumor cells or tumors to 3-BrPA, and methods and compositions useful for identifying tumor cells and tumors that have increased likelihood of sensitivity to 3-BrPA, are described in detail herein (see, e.g., Section II). Methods and compositions analogous to those described in Section II are provided for other transporters and/or transporter-dependent toxic agents, e.g., candidate anti-tumor agents identified as described herein. In some embodiments, the present disclosure provides a method of treating a subject in need of treatment for a tumor, the method comprising: (a) determining that the tumor expresses an increased level of a transporter; and (b) treating the subject with a transporter-dependent toxic agent that is taken up by the transporter of step (a). In some embodiments, the disclosure provides a method of treating a subject in need of treatment for a tumor, the method comprising: (a) identifying a transporter that is expressed at an increased level by the tumor; and (b) treating the subject with a transporter-dependent toxic agent that is taken up by the transporter of step (a). In some embodiments, the tumor is assessed for expression of at least 1, 2, 3, 5, 10, or more different transporters. The assessment may comprise measuring the level of mRNA encoding the transporter or measuring the level of the transporter using, e.g., an ELISA assay, IHC, or other suitable methods such as those described in Section II. A transporter-dependent anti-tumor agent is selected based at least in part on such assessment.

In some embodiments a set of detection reagents suitable for detecting the expression level of multiple genes encoding different transporters is used. In some embodiments the set of detection reagents is suitable for multiplexed detection of multiple mRNAs encoding different transporters or multiplexed detection of different transporters. The detection reagents may be appropriately selected so as to not interfere with each other and/or to allow quantitative determination of relative expression levels. In some embodiments the set of detection reagents are provided in a kit. The detection reagents may be provided in separate containers or at least some of the detection reagents may be provided together in a single composition.

In some embodiments various methods described in the present disclosure comprise measuring one or more characteristics of a cell or tumor such as cell survival or proliferation, glycolytic activity, expression level of one or more genes, activity of one or more gene products, or tumor size or growth rate. In some embodiments one or more cells, biological samples, or tumors are contacted with an agent or combination of agents and one or more characteristics such as cell survival or proliferation, glycolytic activity, expression level of one or more genes, activity of one or more gene products, or tumor size or growth rate is measured. In some embodiments a classification or prediction of 3-BrPA sensitivity based on MCT1 expression may be confirmed by directly measuring the effect of 3-BrPA on tumor cell survival or proliferation or on tumor size or growth rate. In some embodiments agents that may be useful in combination with 3-BrPA (such as those described in Section IV below) may be tested in part by determining the effect of such combination on one or more tumor cell or tumor characteristics.

In some embodiments cells are maintained and/or contacted with one or more agents in vitro (in culture). Cultured cells can be maintained in a suitable cell culture vessel under appropriate conditions (e.g., appropriate temperature, gas composition, pressure, humidity) and in appropriate culture medium. Methods, culture media, and cell culture vessels (e.g., plates (dishes), wells, flasks, bottles, tubes, or other chambers) suitable for culturing cells are known to those of ordinary skill in the art. Typically the vessels contain a suitable tissue culture medium, and the test agent(s) are present in the tissue culture medium, e.g., test agent(s) are added to the culture medium before or after the medium is placed in the culture vessels. One of ordinary skill in the art can select a medium appropriate for culturing a particular cell type. In some embodiments a medium is a chemically defined medium. In some embodiments a medium is free or essentially free of serum or tissue extracts. In some embodiments serum or tissue extract is present. In some embodiments cells are non-adherent. In some embodiments cells are adherent. Such cells may, for example, be cultured on a plastic or glass surface, which may in some embodiments be processed to render it suitable for mammalian cell culture. In some embodiments cells are cultured on or in a material comprising collagen, laminin, Matrigel®, or a synthetic polymer or other material that is intended to provide an environment that resembles in at least some respects the extracellular environment, e.g., extracellular matrix, found in certain tissues in vivo.

In some embodiments mammalian cells are used. In some embodiments mammalian cells are primate cells (human cells or non-human primate cells), rodent (e.g., mouse, rat, rabbit, hamster) cells, canine, feline, bovine, or other mammalian cells. In some embodiments avian cells are used. A cell may be a primary cell, immortalized cell, normal cell, abnormal cell, tumor cell, non-tumor cell, etc., in various embodiments. A cell may originate from a particular tissue or organ of interest or may be of a particular cell type. Primary cells may be freshly isolated from a subject or may have been passaged in culture a limited number of times, e.g., between 1-5 times or undergone a small number of population doublings in culture, e.g., 1-5 population doublings. In some embodiments a cell is a member of a population of cells, e.g., a member of a non-immortalized or immortalized cell line. In some embodiments, a “cell line” refers to a population of cells that has been maintained in culture for at least 10 passages or at least 10 population doublings. In some embodiments, a cell line is derived from a single cell. In some embodiments, a cell line is derived from multiple cells. In some embodiments, cells of a cell line are descended from a cell or cells originating from a single sample (e.g., a sample obtained from a tumor) or individual. A cell may be a member of a cell line that is capable of prolonged proliferation in culture, e.g., for longer than about 3 months (with passaging as appropriate) or longer than about 25 population doublings). A non-immortalized cell line may, for example, be capable of undergoing between about 20-80 population doublings in culture before senescence. In some embodiments, a cell line is capable of indefinite proliferation in culture (immortalized). An immortalized cell line has acquired an essentially unlimited life span, i.e., the cell line appears to be capable of proliferating essentially indefinitely. For purposes hereof, a cell line that has undergone or is capable of undergoing at least 100 population doublings in culture may be considered immortal. In some embodiments, cells are maintained in culture and may be passaged or allowed to double once or more following their isolation from a subject (e.g., between 2-5, 5-10, 10-20, 20-50, 50-100 times, or more) prior to use in a method disclosed herein. In some embodiments, cells have been passaged or permitted to double no more than 1, 2, 5, 10, 20, or 50 times following isolation from a subject prior to use in a method described herein. If desired, cells may be tested to confirm whether they are derived from a single individual or a particular cell line by any of a variety of methods known in the art such as DNA fingerprinting (e.g., short tandem repeat (STR) analysis) or single nucleotide polymorphism (SNP) analysis (which may be performed using, e.g., SNP arrays (e.g., SNP chips) or sequencing).

Numerous tumor cell lines and non-tumor cell lines are known in the art and may be used in various methods described herein. Cell lines can be generated using methods known in the art or obtained, e.g., from depositories or cell banks such as the American Type Culture Collection (ATCC), Coriell Cell Repositories, Deutsche Sammlung von Mikroorganismen and Zellkulturen (German Collection of Microorganisms and Cell Cultures; DSMZ), European Collection of Cell Cultures (ECACC), Japanese Collection of Research Bioresources (JCRB), RIKEN, Cell Bank Australia, etc. The paper and online catalogs of the afore-mentioned depositories and cell banks are incorporated herein by reference. Cells or cell lines may be of any cell type or tissue of origin in various embodiments. Tumor cells or tumor cell lines may be of any tumor type or tissue of origin in various embodiments. Exemplary tumor cell lines and tumors are described in the Examples. In some embodiments a tumor cell or tumor cell line expresses a transporter, e.g., the tumor cell or tumor cell line has increased expression of a transporter. In some embodiments the transporter is MCT1. In some embodiments tumor cells, e.g., a tumor cell line, originates from a human tumor. In some embodiments tumor cells, e.g., a tumor cell line, originates from a tumor of a non-human animal. In some embodiments tumor cells originate from a naturally arising tumor (i.e., a tumor that was not intentionally induced or generated for, e.g., experimental purposes). In some embodiments a tumor cell line originates from a primary tumor. In some embodiments a tumor cell line originates from a metastatic tumor. In some embodiments a tumor cell line originates from a metastasis. In some embodiments a cell line has become spontaneously immortalized in cell culture. In some embodiments a tumor cell line is capable of giving rise to tumors when introduced into an immunocompromised host, e.g., an immunocompromised rodent such as an immunocompromised mouse.

In some embodiments tumor cells are experimentally produced tumor cells. Tumor cells can be produced by genetically modifying a non-tumor cell, e.g., a non-tumor somatic cell, e.g., by expressing or activating an oncogene in the non-tumor cell and/or inactivating or inhibiting expression of one or more tumor suppressor genes (TSG) or inhibiting activity of a gene product of a TSG. Certain experimentally produced tumor cells and exemplary methods of producing tumor cells are described in PCT/US2000/015008 (WO/2000/073420), in U.S. Ser. No. 10/767,018, in Elenbaas, et al., Genes and Development, 15(1):50-65, (2001); and/or Yang, J, et al., Cell 117, 927-939 (2004). In certain embodiments a non-immortal cell, e.g., a non-tumor cell, is immortalized by causing the cell to express telomerase catalytic subunit (e.g., human telomerase catalytic subunit; hTERT). In some embodiments a tumor cell is produced from a non-tumor cell by introducing one or more expression construct(s) or expression vector(s) comprising an oncogene into the cell or modifying an endogenous gene (proto-oncogene) by a targeted insertion into or near the gene or by deletion or replacement of a portion of the gene. For example, cells, e.g., non-tumor cells, can be immortalized with hTERT and transformed by expression of SV40 large T oncoprotein and oncogenic HRAS (e.g., H-rαsV12). In some embodiments a TSG is knocked out or functionally inactivated using gene targeting. For example, a portion of a TSG may be deleted or the TSG may be disrupted by an insertion. In some embodiments a TSG is inhibited by introducing into a cell one or more expression construct(s) or expression vector(s) encoding an inhibitory molecule (e.g., an RNAi agent such as a shRNA or a dominant negative or a negative regulator) that is capable of inhibiting the expression or activity of an expression product of a TSG. Oncogenes and/or TSG inhibitory molecules may be expressed under control of suitable regulatory elements, which may be constitutive or regulatable (e.g., inducible). In some embodiments tumor cells may be produced by expressing or activating multiple oncogenes and/or inhibiting or inactivating multiple TSGs, e.g., 1, 2, 3, 4, or more oncogenes and/or 1, 2, 3, 4, or more TSGs. Many combinations of oncogenes and/or TSGs whose expression/activation or inhibition/inactivation, respectively, can be used to induce tumors are known in the art. Suitable vectors and methods useful for producing genetically engineered tumor cells will be apparent to those of ordinary skill in the art.

The term “oncogene” encompasses nucleic acids that, when expressed, can increase the likelihood of or contribute to cancer initiation or progression. Normal cellular sequences (“proto-oncogenes”) can be activated to become oncogenes (sometimes termed “activated oncogenes”) by mutation and/or aberrant expression. In various embodiments an oncogene can comprise a complete coding sequence for a gene product or a portion that maintains at least in part the oncogenic potential of the complete sequence or a sequence that encodes a fusion protein. Oncogenic mutations can result, e.g., in altered (e.g., increased) protein activity, loss of proper regulation, or an alteration (e.g., an increase) in RNA or protein level. Aberrant expression may occur, e.g., due to chromosomal rearrangement resulting in juxtaposition to regulatory elements such as enhancers, epigenetic mechanisms, or due to amplification, and may result in an increased amount of proto-oncogene product or production in an inappropriate cell type. As known in the art, proto-oncogenes often encode proteins that control or participate in cell proliferation, differentiation, and/or apoptosis. These proteins include, e.g., various transcription factors, chromatin remodelers, growth factors, growth factor receptors, signal transducers, and apoptosis regulators. Oncogenes also include a variety of viral proteins, e.g., from viruses such as polyomaviruses (e.g., SV40 large T antigen) and papillomaviruses (e.g., human papilloma virus E6 and E7). A TSG may be any gene wherein a loss or reduction in function of an expression product of the gene can increase the likelihood of or contribute to cancer initiation or progression. Loss or reduction in function can occur, e.g., due to mutation or epigenetic mechanisms. Many TSGs encode proteins that normally function to restrain or negatively regulate cell proliferation and/or to promote apoptosis. In some embodiments an oncogene or TSG encodes a miRNA. Exemplary oncogenes include, e.g., MYC, SRC, FOS, JUN, MYB, RAS, RAF, ABL, ALK, AKT, TRK, BCL2, WNT, HER2/NEU, EGFR, MAPK, ERK, MDM2, CDK4, GLI1, GLI2, IGF2, TP53, etc. Exemplary TSGs include, e.g., RB, TP53, APC, NF1, BRCA1, BRCA2, PTEN, CDK inhibitory proteins (e.g., p16, p21), PTCH, WT1, etc. It will be understood that a number of these oncogene and TSG names encompass multiple family members and that many other TSGs are known.

Cells, e.g., tumor cells, may be maintained in a culture medium comprising an agent of interest. The effect of the agent on tumor cell viability, proliferation, tumor-initiating capacity, glycolytic activity, or any other tumor cell property may be measured using any suitable method known in the art in various embodiments. In certain embodiments survival and/or proliferation of a cell or cell population may be determined by a cell counting assay (e.g., using visual inspection, automated image analysis, flow cytometer, etc.), a replication assay, a cell membrane integrity assay, a cellular ATP-based assay, a mitochondrial reductase activity assay, a BrdU, EdU, or H3-Thymidine incorporation assay, calcein staining, a DNA content assay using a nucleic acid dye, such as Hoechst Dye, DAPI, Actinomycin D, 7-aminoactinomycin D or propidium iodide, a cellular metabolism assay such as resazurin (sometimes known as AlamarBlue or by various other names), MTT, XTT, and CellTitre Glo, etc., a protein content assay such as SRB (sulforhodamine B) assay; nuclear fragmentation assays; cytoplasmic histone associated DNA fragmentation assay; PARP cleavage assay; TUNEL staining; or annexin staining. In some embodiments an assay may reflect two or more characteristics. For example, the CyQUANT® family of cell proliferation assays (Life Technologies) are based on both DNA content and membrane integrity. In some embodiments cell survival or proliferation is assessed by measuring expression of one or more genes that encode gene products that mediate cell survival or proliferation or cell death, e.g., genes that encode products that play roles in or regulate the cell cycle or cell death (e.g., apoptosis). Examples of such genes include, e.g., cyclin dependent kinases, cyclins, BAX/BCL2 family members, caspases, etc. One of ordinary skill in the art will be able to select appropriate genes to be used as indicators of cell survival or proliferation. It will be understood that in some embodiments an assay of cell survival and/or proliferation may determine cell number, e.g., number of living cells, and may not distinguish specifically between cell survival per se and cell proliferation, e.g., the assay result may reflect a combination of survival and proliferation. In some embodiments an assay able to specifically assess survival or proliferation or cell death (e.g., apoptosis or necrosis) may be used.

In some embodiments an agent or combination of agents is tested to determine whether it has an anti-tumor effect or to quantify an anti-tumor effect. For example, in some embodiments the effect of 3-BrPA and a second glycolysis inhibitor is assessed. In some embodiments the effect of a candidate glycolysis modulator identified as described in Section IV is assessed. In some embodiments the effect of a molecule that is taken up via MCT1-mediated transport is assessed. In some embodiments the effect of an agent or combination of agents is tested on tumor cells and non-tumor cells, e.g., to determine whether the agent is selectively toxic to tumor cells or to measure the degree of selectivity.

In some embodiments an anti-tumor effect is inhibition of tumor cell survival or proliferation. It will be understood that inhibition of cell proliferation or survival by an agent or combination of agents may, or may not, be complete. For example, cell proliferation may, or may not, be decreased to a state of complete arrest for an effect to be considered one of inhibition or reduction of cell proliferation. In some embodiments, “inhibition” may comprise inhibiting proliferation of a cell that is in a non-proliferating state (e.g., a cell that is in the GO state, also referred to as “quiescent”) and/or inhibiting proliferation of a proliferating cell (e.g., a cell that is not quiescent). Similarly, inhibition of cell survival may refer to killing of a cell, or cells, such as by causing or contributing to necrosis or apoptosis, and/or the process of rendering a cell susceptible to death, e.g., causing or increasing the propensity of a cell to undergo apoptosis or necrosis. The inhibition may be at least about 10%, 15%, 20%, 25%, 30%, 35%, 40%, 45%, 50%, 55%, 60%, 65%, 70%, 75%, 80%, 85%, 90%, 95%, 99%, or 100% of a reference level (e.g., a control level).

In some embodiments an anti-tumor effect is inhibition of the capacity of tumor cells to form colonies in suspension culture. In some embodiments an anti-tumor effect is inhibition of capacity of the one or more tumor cells to form colonies in a semi-solid medium such as soft agar or methylcellulose. In some embodiments an anti-tumor effect is inhibition of capacity of the one or more tumor cells to form tumor spheres in culture. In some embodiments an anti-tumor effect is inhibition of the capacity of the one or more tumor cells to form tumors in vivo.

In some embodiments sensitivity of a tumor cell, tumor cell line, or tumor to an agent or combination of agents, is assessed using an in vivo tumor model. An “in vivo” tumor model involves the use of one or more living non-human animals (“test animals”). For example, an in vivo tumor model may involve administration of an agent and/or introduction of tumor cells to one or more test animals. In some embodiments a test animal is a mouse, rat, or dog. Numerous in vivo tumor models are known in the art. By way of example, certain in vivo tumor models are described in U.S. Pat. No. 4,736,866; U.S. Ser. No. 10/990,993; PCT/US2004/028098 (WO/2005/020683); and/or PCT/US2008/085040 (WO/2009/070767). Introduction of one or more cells into a subject (e.g., by injection or implantation) may be referred to as “grafting”, and the introduced cell(s) may be referred to as a “graft”. In general, any tumor cells may be used in an in vivo tumor model in various embodiments. Tumor cells may be from a tumor cell line or tumor sample. In some embodiments tumor cells originate from a naturally arising tumor (i.e., a tumor that was not intentionally induced or generated for, e.g., experimental purposes). In some embodiments experimentally produced tumor cells may be used. The number of tumor cells introduced may range, e.g., from 1 to about 10, 10², 10³, 10⁴, 10⁵, 10⁶, 10⁷,10⁸, 10⁹, or more. In some embodiments at least some of the tumor cells have increased expression of a transporter, e.g., MCT1. In some embodiments the tumor cells are from a tumor cell line or tumor that naturally has increased expression of the transporter. In some embodiments the tumor cells are selected or genetically modified to have increased expression of the transporter In some embodiments the tumor cells are of the same species or inbred strain as the test animal. In some embodiments tumor cells may originate from the test animal. In some embodiments the tumor cells are of a different species than the test animal. For example, the tumor cells may be human cells. In some embodiments, a test animal is immunocompromised, e.g., in certain embodiments in which the tumor cells are from a different species to the test animal or originate from an immunologically incompatible strain of the same species as the test animal. For example, a test animal may be selected or genetically engineered to have a functionally deficient immune system or may subjected to radiation or an immunosuppressive agent or surgery such as removal of the thymus) so as to reduce immune system function. In some embodiments, a test animal is a SCID mouse, NOD mouse, NOD/SCID mouse, nude mouse, and/or Rag1 and/or Rag2 knockout mouse, or a rat having similar immune system dysfunction. Tumor cells may be introduced at an orthotopic or non-orthotopic location. In some embodiments tumor cells are introduced subcutaneously, under the renal capsule, or into the bloodstream. Non-tumor cells (e.g., fibroblasts, bone marrow derived cells), an extracellular matrix component or hydrogel (e.g., collagen or Matrigel®), or an agent that promotes tumor development or growth may be administered to the test animal prior to, together with, or separately from the tumor cells.

In some embodiments tumor cells are contacted with an agent, e.g., 3-BrPA or an analog thereof, optionally in combination with a second agent, prior to grafting (in vitro) and/or following grafting (by administering the agent to the test animal). The agent may be administered to the test animal at around the same time as the tumor cells, and/or at one or more subsequent times. The number, size, growth rate, metastasis, or other properties of resulting tumors (if any) may be assessed at one or more time points following grafting and, if desired, may be compared with a control in which tumor cells of the same type are grafted without contacting them with the agent or using a higher or lower concentration or dose of the agent.

In some embodiments a tumor arises due to neoplastic transformation that occurs in vivo, e.g., at least in part as a result of one or more mutations in a cell in a subject. In some embodiments a test animal is a tumor-prone animal. The test animal may, for example, be of a species or strain that naturally has a predisposition to develop tumors and/or may be a genetically modified tumor-prone animal. For example, in some embodiments the animal is a genetically engineered animal at least some of whose cells comprise, as a result of genetic modification, at least one activated oncogene and/or in which at least one tumor suppressor gene has been functionally inactivated. Standard methods of generating genetically modified animals, e.g., transgenic animals that comprises exogenous genes or animals that have an alteration to an endogenous gene, e.g., an insertion or an at least partial deletion or replacement (sometimes referred to as “knockout” or “knock-in” animal) can be used.

Any of a wide variety of methods and/or devices known in the art may be used to assess tumors in vivo. Tumor number, size, growth rate, or metastasis may, for example, be assessed using various imaging modalities, e.g., 1, 2, or 3-dimensional imaging (e.g., using X-ray, CT scan, ultrasound, or magnetic resonance imaging, etc.) and/or functional imaging (e.g., PET scan) may be used to detect or assess lesions (local or metastatic), e.g., to measure anatomical tumor burden, detect new lesions (e.g., metastases), etc. In some embodiments PET scanning with the glucose analog fluorine-18 (F-18) fluorodeoxyglucose (FDG) as a tracer is used. As known in the art, FDG is taken up and phosphorylated by glucose-using cells. FDG remains trapped in cells that take it up until it decays, which results in intense radiolabeling of tissues with high glucose uptake, such as the brain, the liver, and certain cancers. In some embodiments tumor(s) may be removed from the body (e.g., at necropsy) and assessed (e.g., tumors may be counted, weighed, and/or size (e.g., dimensions) measured). In some embodiments the size and/or number of tumors may be determined non-invasively. For example, in certain tumor models, tumor cells that are fluorescently labeled (e.g., by expressing a fluorescent protein such as GFP) can be monitored by various tumor-imaging techniques or instruments, e.g., non-invasive fluorescence methods such as two-photon microscopy. The size of a tumor implanted or developing subcutaneously can be monitored and measured underneath the skin. In certain embodiments a tumor is considered sensitive to an agent, e.g., 3-BrPA, if the growth rate or size (e.g., estimated volume or weight) of the tumor is reduced by at least 50%, 60%, 70%, 80%, 90%, 95%, or more, by treatment at a dose (or series of doses) that are tolerated by a subject. In certain embodiments a tumor is rendered undetectable. In some embodiments recurrence is prevented for at least a period of time. In some embodiments a reduction in tumor growth rate or size or prevention of recurrence is maintained at least while treatment is continued. In some embodiments such reduction or prevention of recurrence is maintained for at least about 3, 4, 6, 8, 12, 16, 24, 36, 44, 52 weeks, or more, e.g., at least about 15, 18, 24 months, 3-5 years, or more. In some embodiments sufficient tumor cells may be eradicated so that the tumor does not recur after cessation of treatment when assessed at least about 3, 4, 6, 8, 12, 16, 24, 36, 44, 52 weeks, or more, e.g., at least about 15, 18, 24 months, 3-5 years, or more, after cessation of treatment.

In some embodiments, treatment sensitivity of a tumor in a human subject may be evaluated at least in part using objective criteria such as the original or revised Response Evaluation Criteria In Solid Tumors (RECIST), a guideline that can be used to objectively determine when or whether cancer patients improve (“respond”), remain about the same (“stable disease”), or worsen (“progressive disease”) based on anatomical tumor burden (e.g., measured using physical examination and/or imaging techniques such as those mentioned above). A response may be either a “complete response” or a “partial response”. The original RECIST guideline is described in Therasse P, et al. J Natl Cancer Inst (2000) 92:205-16. A revised RECIST guideline (Version 1.1) is described in Eisenhauer, E., et al., Eur J. Cancer. (2009) 45(2):228-47). In the case of brain tumors, response assessment (e.g., in high-grade gliomas such as glioblastoma) can use the Macdonald criteria (Macdonald D, et al. (1990) Response criteria for phase II studies of supratentorial malignant glioma. J Clin Oncol 8:1277-1280), e.g., as extrapolated to magnetic resonance imaging (MRI) (Rees J (2003) Advances in magnetic resonance imaging of brain tumours. Curr Opin Neurol 16:643-650). An updated version of the Macdonald criteria may be used (Wen, P Y, et al., J Clin Oncol. (2010) 28(11):1963-72). In the case of lymphomas or leukemias, response criteria known in the art can be used (see, e.g., Cheson B D, et al. Revised response criteria for malignant lymphoma. J Clin Oncol 2007; 10:579-86). It will be appreciated that the guidelines and criteria mentioned herein for assessing tumor sensitivity are merely exemplary. Modified or updated versions thereof or other reasonable criteria (e.g., as determined by a person of ordinary skill in the art) may be used. Clinical assessment of symptoms or signs associated with tumor presence, stage, regression, progression, or recurrence may be used. In certain embodiments criteria based on anatomic tumor burden should reasonably correlate with a clinically meaningful benefit such as increased survival (e.g., increased progression-free survival, increased cancer-specific survival, or increased overall survival) or at least improved quality of life such as reduction in one or more symptoms. In some embodiments a response lasts for at least 2, 3, 4, 5, 6, 8, 12 months, or more. In some embodiments tumor response or recurrence may be assessed at least in part by testing a sample comprising a body fluid such as blood for the presence of tumor cells and/or for the presence or level or change in level of one or more substances (e.g., microRNA, protein) produced or secreted by tumor cells. For example, prostate specific antigen (PSA) and carcinoembryonic antigen (CEA) are two such markers. The extracellular domain of HER2 can be shed from the surface of tumor cells and enter the circulation. A normal level or a reduction in level over time of one or more substances derived from tumor cells may indicate a response or maintenance of remission. An abnormally high level or an increase in level over time may indicate progression or recurrence.

In some embodiments, treatment sensitivity of a tumor in a subject, e.g., a human subject, is assessed by evaluating survival, e.g., 3 month or 6 month survival, or 1, 2, 5, or 10 year survival. In some embodiments, overall survival is assessed. In some embodiments disease-specific survival (i.e., survival considering only mortality due to cancer) is assessed. In some embodiments, progression-free survival is assessed. In some embodiments, a tumor is considered sensitive to a compound If treatment with the compound results in an increased survival relative to predicted survival in the absence of treatment. In some embodiments, a tumor is considered sensitive to a compound If adding the compound to a cancer treatment regimen results in an increased survival relative to predicted survival using the same cancer regimen but without the compound. In some embodiments, a tumor is considered sensitive to a compound (e.g., 3-BrPA) if using the compound in place of a different compound in a standard or experimental cancer treatment regimen results in an increased response, e.g., increased survival, relative to predicted survival using the standard or experimental cancer treatment regimen.

In some embodiments, a difference between two or more measurements or between two or more groups of samples or subjects is statistically significant as determined using an appropriate statistical test or analytical method. One of ordinary skill in the art will be able to select an appropriate statistical test or analytical method for evaluating statistical significance. In some embodiments, a difference between two or more measurements or between two or more groups of subjects would be considered clinically meaningful or clinically significant by one of ordinary skill in the art. In some embodiments statistically significant refers to a P-value of less than 0.05, e.g., less than 0.025, e.g., less than 0.01, e.g., less than 0.005. In some embodiments a P-value is a two-tailed P-value.

In some embodiments of any aspect or embodiment in the present disclosure relating to cells, a population of cells, cell sample, or similar terms, the number of cells is between 10 and 10¹³ cells. In some embodiments the number of cells may be at least about 10³, 10⁴, 10⁵, 10⁶, 10⁷, 10⁸, 10⁹, 10¹⁰, 10¹¹, 10¹² cells, or more. In some embodiments, the number 10¹⁰, of cells is between 10⁵ and 10¹² cells, e.g., at least 10⁶, 10⁷, 10⁸, 10⁹, 10¹⁰, 10¹¹, up to about 10¹² or about 10¹³. In some embodiments a screen is performed using multiple populations of cells and/or is repeated multiple times. In some embodiments, the number of cells is between 10⁵ and 10¹² cells, e.g., at least 10⁶, 10⁷, 10⁸, 10⁹, 10¹⁰, 10¹¹, up to about 10¹². In some embodiments smaller numbers of cells are of use, e.g., between 1-10⁴ cells. In some embodiments a population of cells is contained in an individual vessel, e.g., a culture vessel such as a culture plate, flask, or well. In some embodiments a population of cells is contained in multiple vessels. In some embodiments two or more cell populations are pooled to form a larger population.

In some embodiments, one or more compound(s) with a desired IC₅₀ or IC₉₀ is identified. In some embodiments, an IC₅₀ and/or IC₉₀ is no greater than 100 mg/ml, e.g., no greater than 10 mg/ml, e.g., no greater than 1.0 mg/ml, e.g., no greater than 100 μg/ml, e.g., no greater than 10 μg/ml, e.g., no greater than 5 μg/ml or no greater than 1 μg/ml. In some embodiments, an IC₅₀ and/or IC₉₀ is less than or equal to 500 μM. In some embodiments, an IC₅₀ and/or IC₉₀ is less than or equal to 100 μm. In some embodiments, an IC₅₀ and/or is IC₉₀ less than or equal to 10 μM. In some embodiments, an IC₅₀ and/or IC₉₀ is in the nanomolar range, i.e., less than or equal to 1 μM. In some embodiments, an IC₅₀ and/or IC₉₀ between 10 nm and 100 nm, between 100 nm and 500 nm, or between 500 nm and 1 μM. In some embodiments a dose response curve is obtained at one or more time points. For example, cells may be exposed to a range of different concentrations, and cell survival or proliferation may be assessed at one or more time points thereafter. An IC₅₀ and/or IC₉₀ may be obtained from a dose response curve using a regression model, e.g., a nonlinear regression model.

### IV. Methods of Identifying Highly Glycolytic Tumors and Glycolytic Modulators

In some aspects, the disclosure provides methods of evaluating the glycolytic activity of a tumor cell, tumor cell line, or tumor. In some aspects, the disclosure provides methods of classifying a tumor cell, tumor cell line, or tumor according to its level of glycolytic activity. Certain of the methods are based on Applicants' identification of distinct sets of genes, the expression of which correlates with either high levels of glycolytic activity or lower levels of glycolytic activity across a panel of 15 cancer cell lines. Glycolytic rate was determined using the ratio of oxygen consumption rate (OCR) to extracellular acidification rate (ECAR), a proxy for lactate production, as a measure of the relative contributions of mitochondrial respiration via oxidative phosphorylation (OXPHOS) and glycolysis to cellular energy production. The measurements were performed using an extracellular flux analyzer (Seahorse Biosciences, Inc.) in RPMI media containing 10 mM glucose. Thus cell lines exhibiting a low OCR/ECAR are considered highly glycolytic, while cell lines exhibiting a higher OCR/ECAR are less glycolytic. In some embodiments “highly glycolytic” or “high level of glycolytic activity” refers to an OCR/ECAR ratio equal to or below 5. In some embodiments a “low level of glycolytic activity” refers to an OCR/ECAR ratio equal to or above 10. In some embodiments an “intermediate level of glycolytic activity” refers to an OCR/ECAR ratio greater than 5 and less than 10. HS578T, CAL-51, and PC-3 are examples of tumor cell lines that were found to be highly glycolytic even under conditions where oxygen is not limiting. In some embodiments a “low level of glycolytic activity” refers to a relatively low level of glycolytic activity by comparison with other tumors or tumor cell lines. A “low level of glycolytic activity” for a tumor or tumor cell line may or may not be low compared with the level of glycolytic activity of non-tumor cells or tissues. MDA-MB-453, BT474, and ZR-75-1 are examples of tumor cell lines that were found not to be highly glycolytic, at least under conditions in which oxygen is not limiting.

Among a set of tumor cell lines tested, most estrogen receptor (ER) positive cells were found to display high levels of OXPHOS, and most ER negative cells were found to be highly glycolytic. Additionally, MCT1 was found to be expressed at high levels in ER negative cells; thus these cells were generally highly sensitive to 3-BrPA. In some embodiments, the disclosure provides the insight that ER negative tumors tend to be highly glycolytic and sensitive to 3-BrPA. In some embodiments, a method of determining whether a subject in need of treatment for a breast tumor is a candidate for treatment with 3-bromopyruvate (3-BrPA) or an analog thereof comprises determining whether the breast tumor is estrogen receptor negative; and identifying the subject as a candidate for treatment with 3-BrPA) or an analog thereof if the tumor is ER negative. In some embodiments a method of treating a subject in need of treatment for a breast tumor comprises: (a) determining that the breast tumor is ER negative; and (b) treating the subject with 3-BrPA or an analog thereof.

As noted above, Applicants identified distinct sets of genes, the expression of which correlates with either high levels of glycolytic activity or lower levels of glycolytic activity. The 40 genes whose expression levels were found to be most highly correlated with high glycolytic activity (low OCR/ECAR) may be referred to herein as “HGAA Genes”, standing for “high glycolytic activity associated genes”, and are listed on the left side of FIG. S4(c) (i.e., left two columns in FIG. S4(c). The 40 genes whose expression levels were found to be most highly correlated with lower levels of glycolytic activity (high OCR/ECAR) may be referred to herein as “LGAA Genes”, standing for “low glycolytic activity associated genes” and are listed on the right side of FIG. S4(c) (i.e., right two columns in FIG. S4(c). Sequences of gene products of the HGAA and LGAA genes are readily available in databases such as those mentioned above. Two glycolytic enzymes (LDHB and PGM1), were amongst the genes whose expression most strongly and significantly correlated with lower OCR/ECAR ratios, as was MCT1. The latter finding indicates that tumors which exhibit the highest rates of glycolysis are more likely to have elevated levels of MCT1 and therefore will be more sensitive to 3-BrPA treatment.

In some aspects, provided herein is a method of classifying a tumor cell, tumor cell line, or tumor according to its level of glycolytic activity, the method comprising: (a) assessing expression of at least one High Glycolytic Activity Associated (HGAA) gene or at least one Low Glycolytic Activity Associated (LGAA) gene in the tumor cell, tumor cell line, tumor, wherein increased expression of HGAA genes is correlated with increased glycolytic activity, and wherein increased expression of LGAA genes is correlated with decreased glycolytic activity; and (b) classifying the tumor cell, tumor cell line, or tumor according to its level of glycolytic activity based on the result of step (a). In some embodiments a method of evaluating the glycolytic activity of a tumor cell, tumor cell line, or tumor comprises: (a) assessing expression of at least one High Glycolytic Activity Associated (HGAA) gene or at least one Low Glycolytic Activity Associated (LGAA) in the tumor cell, tumor cell line, or tumor, wherein increased expression of HGAA genes is correlated with increased glycolytic activity, and wherein increased expression of LGAA genes is correlated with decreased glycolytic activity; and (b) comparing the result of step (a) with a reference, wherein the result of the comparison is indicative of the level of glycolytic activity of the tumor cell, tumor cell line, or tumor. In some embodiments the reference is a gene expression profile of a tumor or tumor cell line with high glycolytic activity or a gene expression profile of a tumor or tumor cell line that lacks high glycolytic activity, e.g., a tumor or tumor cell line that has low glycolytic activity, wherein the gene expression profile includes measurements of expression of at least one HGAA gene and/or at least one LGAA gene. In some embodiments the comparison comprises determining the correlation between the gene expression profile of a tumor cell, tumor cell line, or tumor and a gene expression profile of a tumor or tumor cell line with high glycolytic activity with respect to the HGAA and LGAA genes whose expression is assessed. A high degree of correlation indicates that the tumor cell, tumor cell line, or tumor has a high level of glycolytic activity, and a low degree of correlation indicates that the tumor cell, tumor cell line, or tumor does not have a high level of glycolytic activity. In some embodiments the comparison comprises determining the correlation between the gene expression profile of the tumor cell, tumor cell line, or tumor and a gene expression profile of a tumor or tumor cell line with low glycolytic activity with respect to the HGAA and LGAA genes whose expression is assessed. A high degree of correlation indicates that the tumor cell, tumor cell line, or tumor has a low level of glycolytic activity, and a low degree of correlation indicates that the tumor cell, tumor cell line, or tumor does not have a low level of glycolytic activity. In some embodiments the reference comprises gene expression profiles from multiple tumors or tumor cell lines having a range of levels of glycolytic activity. For example, in some embodiments the gene expression profiles are from a panel of at least 2, 3, 5, 10, 15, 20, 25, 30, 40, 50, 75, 100, or more tumor cell lines, wherein at least one member of the panel has a high level of glycolytic activity and at least one member of the panel has a low level of glycolytic activity. In some embodiments at least one member of the panel has an intermediate level of glycolytic activity. In some embodiments the comparison comprises performing a cluster analysis based on gene expression profiles of the tumor cell, tumor cell line, or tumor and gene expression profiles from multiple tumors or tumor cell lines having a range of levels of glycolytic activity and determining whether the tumor cell, tumor cell line, or tumor clusters with highly glycolytic tumors or with tumors that are not highly glycolytic, e.g., tumors that have intermediate or low levels of glycolytic activity. The cluster analysis is performed with respect to the HGAA and LGAA genes whose expression is assessed. Methods of performing correlation analysis or cluster analysis will be apparent to those of ordinary skill in the art. In some embodiments hierarchical clustering or k-means clustering is performed.

In some embodiments of any of the methods at least 2, 3, 4, 5, 10, 15, 20, 25, 30, 35, or 40 HGAA genes are assessed. In some embodiments of any of the methods at least 2, 3, 4, 5, 10, 15, 20, 25, 30, 35, or 40 LGAA genes are assessed. In some embodiments of any of the methods at least 2, 3, 4, 5, 10, 15, 20, 25, 30, 35, or 40 HGAA genes and at least 2, 3, 4, 5, 10, 15, 20, 25, 30, 35, or 40 LGAA genes are assessed. In some embodiments of any of the methods, at least the top 2, 3, 4, 5, 10, 15, 20, or 25 HGAA genes, at least the top 2, 3, 4, 5, 10, 15, 20, or 25 LGAA genes, or at least the top 2, 3, 4, 5, 10, 15, 20, or 25 HGAA genes and at least the top 2, 3, 4, 5, 10, 15, 20, or 25 LGAA genes are assessed. In some embodiments of any of the methods the HGAA genes whose expression is assessed include at least one HGAA gene that is not LDHB or PGM1. The expression level can be assessed using any method known in the art and may comprise measuring RNA or protein. Examples of such methods are discussed above.

Methods of evaluating tumor cell, tumor cell line, or tumor glycolytic activity or classifying a tumor cell, tumor cell line, or tumor according to glycolytic activity may further comprise performing one or more steps to apply the method for one or more purpose(s). In some embodiments a method is used to identify a highly glycolytic tumor and thereby identify a tumor that has an increased likelihood of responding to treatment with a glycolysis inhibitor. In some embodiments the method further comprises treating a subject with a glycolysis inhibitor, wherein the subject is in need of treatment for a tumor that has been identified as highly glycolytic based at least in part on assessing expression of at least one HGAA gene and/or at least one LGAA gene. In some embodiments a method is used to identify a highly glycolytic tumor and thereby identify a tumor that has an increased likelihood of responding to treatment with 3-BrPA or an analog thereof. In some embodiments the method further comprises treating a subject with 3-BrPA or an analog thereof, wherein the subject is in need of treatment for a tumor that has been identified as highly glycolytic based at least in part on assessing expression of at least one HGAA gene and/or at least one LGAA gene.

In some embodiments a method is used to correlate the level of tumor glycolytic activity with a characteristic of interest such as outcome or response to one or more pharmacological or non-pharmacological therapies. In some embodiments the method may be used, for example, to examine efficacy of an agent or combination in treating highly glycolytic tumors, e.g., as compared with its efficacy in treating tumors that are not highly glycolytic, or as compared with its overall efficacy. In some embodiments the method may be used, for example, to examine efficacy of an agent or combination in treating tumors that have a low level of glycolytic activity e.g., as compared with its efficacy in treating tumors that are not highly glycolytic, or as compared with its overall efficacy. “Overall efficacy” in this context refers to efficacy in treating tumors without regard for level of glycolytic activity (e.g., tumors in general, or tumors of a type or subtype for which an agent or combination would ordinarily be used). The method may, for example, employ gene expression data obtained from databases for which outcome or treatment response information is available, gene expression data obtained from archived tumor samples for which outcome or treatment response information is available or prospectively, e.g., in a study in which samples are obtained at least in part for the purpose of assessing expression of at least one HGAA gene and/or at least one LGAA gene and correlating the result with outcome or treatment response. In some embodiments at least one of the drugs is a glycolysis inhibitor.

In some embodiments a method is used to determine the level of effectiveness of an agent or combination in treating tumors that have a high, intermediate, or low level of glycolytic activity. In some embodiments a method is used to identify an agent or combination the efficacy of which correlates with the level of glycolytic activity of a tumor. In some embodiments the method may be used to identify an agent or combination the efficacy of which differs (e.g., is higher or lower) in tumors having high levels of glycolytic activity as compared with the overall efficacy of the agent or combination. In some embodiments the method may be used to identify an agent or combination the efficacy of which differs (e.g., is higher or lower) in tumors having low levels of glycolytic activity as compared with the overall efficacy of the agent or combination. In some embodiments a method is used to identify an agent or combination that has higher efficacy in treating highly glycolytic tumors than its overall efficacy. In some embodiments a method is used to identify an agent or combination that has lower efficacy in treating highly glycolytic tumors than its overall efficacy. In some embodiments a method is used to identify an agent or combination that has higher efficacy in treating tumors that have a low level of glycolytic activity than its overall efficacy. In some embodiments a method is used to identify an agent or combination that has lower efficacy in treating tumors that have a low level of glycolytic activity than its overall efficacy.

In some embodiments a method may be used to identify an agent or combination of agents the efficacy of which differs (e.g., is higher or lower) in tumors having high levels of glycolytic activity as compared with the efficacy in tumors not having high levels of glycolytic activity. In some embodiments a method is used to identify an agent or combination of agents that has higher efficacy in treating highly glycolytic tumors than in treating tumors that are not highly glycolytic, e.g., tumors that have a low level of glycolytic activity. In some embodiments a method is used to identify an agent or combination of agents that has a higher or efficacy in treating tumors that have a low level of glycolytic activity than in treating tumors that are not highly glycolytic, e.g., tumors that have a low level of glycolytic activity.

Once agents or combinations whose efficacy correlates with tumor glycolytic activity are identified this information can be used in selecting an appropriate therapy for a subject based on tumor glycolytic level. For example, an agent that has higher efficacy for treating highly glycolytic tumors than its overall efficacy may be selected for treatment of a highly glycolytic tumor or an agent that has lower efficacy for treating highly glycolytic tumors than its overall efficacy may be replaced or supplemented by an agent that has higher efficacy for highly glycolytic tumors. In some embodiments an agent that has lower efficacy for treating highly glycolytic tumors than its overall efficacy may be avoided, e.g., if there are suitable alternatives. Similar methods may be used in selecting agents for treating a tumor that has a low or intermediate level of glycolytic activity.

In some embodiments the effect of an agent on glycolytic activity is evaluated by determining the effect of the agent on expression of one or more HGAA and/or LGAA genes. For example, cells are contacted with an agent in culture for a suitable time period, after which expression of one or more HGAA and/or one more LGAA genes is assessed. The level of glycolytic activity is determined based on the expression levels. In some embodiments expression level(s) are compared with expression level(s) obtained from control cells (e.g., cells of the same type or cell line) not contacted with the agent. The effect of the agent on the level of glycolytic activity of the cells is determined based on the expression of the HGAA and/or LGAA gene(s). In some embodiments, if cells cultured in the presence of the agent exhibit lower glycolytic activity than control cells the agent is identified as a candidate glycolysis inhibitor or as a candidate OXPHOS enhancer. In some embodiments, if cells cultured in the presence of the agent exhibit higher glycolytic activity than control cells the agent is identified as a candidate glycolysis enhancer or as a candidate OXPHOS inhibitor. In some embodiments the cells are cancer cells. In some embodiments the cells are highly glycolytic cells, e.g., highly glycolytic cancer cells.

In some embodiments a method may be used to identify new glycolysis modulators, e.g., glycolysis inhibitors or glycolysis enhancers, or to identify new OXPHOS modulators, e.g., OXPHOS inhibitors or OXPHOS enhancers. Glycolysis inhibitors or OXPHOS inhibitors may be useful as anti-cancer agents. Glycolysis enhancers may be useful for treatment of diseases characterized by impaired glycolysis (e.g., disorders arising from glycolytic enzyme defects) or in other situations in which increased glycolysis may be beneficial or desired, such as situations in which ATP production via oxidative phosphorylation is impaired. Glycolysis enhancers or OXPHOS enhancers may be useful for treatment of diseases characterized by impaired oxidative phosphorylation (e.g., disorders arising from defects in OXPHOS components) or in other situations in which increased ATP production may be beneficial or desired. In some embodiments highly glycolytic cells may be used in a screen in which it is of particular interest to identify glycolysis inhibitors and/or OXPHOS enhancers. In some embodiments cells that have a low level of glycolysis may be used in a screen in which it is of particular interest to identify glycolysis enhancers and/or OXPHOS inhibitors.

Methods of performing screens, and test agents suitable for use in screens, are described in Section III. Test agents that are identified as “hits” in a screen may be retested using the same assay or different assays. For example, a candidate glycolysis inhibitor can be tested to determine its effect on OCR/ECAR or may be tested to determine its effect on tumor cell survival or proliferation in culture or in an in vivo tumor model. Additional compounds, e.g., analogs, that have a desired activity or improved property can be identified or designed based on compounds identified in a screen, as discussed in Section III.

In some embodiments, existing gene expression data that has been generated in one or more experiments in which cells are contacted with one or more test agents is obtained, e.g., from a database. The gene expression data is analyzed to determine whether a test agent altered the glycolytic activity of the cells, as determined based on change in expression levels of one or more HGAA genes or LGAA genes in cells contacted with the test agent. Examples of databases that contain large quantities of gene expression data available to the public include the Gene Expression Omnibus available at URL http://www.ncbi.nlm.nih.gov/geo/) (Barrett T, et al., NCBI GEO: archive for functional genomics data sets—10 years on; Nucleic Acids Res. 2011 January; 39 (Database issue):D1005-10; ArrayExpress at EBI or the Gene Expression Atlas (both available at URL http://www.ebi.ac.uk/arrayexpress/); the Stanford Microarray Database available at URL http://smd.stanford.edu/ (Hubble J, et al. Implementation of GenePattern within the Stanford Microarray Database. Nucleic Acids Res 2009 Jan. 1; 37 (Database Issue):D898-901); ArrayTrack™ available at URL http://www.fda.gov/ScienceResearch/BioinformaticsTools/Arraytrack//, Oncomine (www.oncomine.com; Rhodes D R et al., Neoplasia. 2007, 9(2):166-80), etc.

An agent identified as a candidate modulator of glycolysis or OXPHOS may be further tested to more directly determine its effect on glycolysis or OXPHOS, e.g., by measuring OCR, ECAR, or a ratio thereof, optionally in the presence of an OXPHOS inhibitor. In some embodiments a candidate modulator of glycolysis is tested to confirm its effect on glycolysis by measuring one or more indicators of glycolysis such as ECAR or OCR/ECAR. In some embodiments a candidate OXPHOS modulator is tested to confirm its effect on OXPHOS by measuring one or more indicators of OXPHOS such as OCR. In some embodiments OCR may be measured in the presence and in the absence of an OXPHOS inhibitor to determine the proportion of OCR due to OXPHOS. In some embodiments one or more indicators of glycolysis or OXPHOS is measured using an extracellular flux analyzer such as the XF24 or XF96 Extracellular Flux Analyzer (Seahorse Bioscience, Billerica, Mass.). In some embodiments one or more such measurements is performed in the presence of a known glycolysis inhibitor or a known inhibitor of mitochondrial respiration such as rotenone to specifically identify the contribution of glycolysis or mitochondrial respiration to a measured value, e.g., OCR. The XF24 system is described in, e.g., Wu M, et al. Am J Physiol Cell Physiol, 2007; 292: C125-136. The XF96 system is similar but permits use of 96-well plates. In some embodiments cell viability is measured in a parallel experiment with substantially identically processed cells using a method that does not rely on ATP production as an indicator of cell viability. For example, calcein AM staining may be used. In some embodiments the rate of oxygen consumption may be determined using Clark electrodes or the rate of extracellular acidification may be determined using a microphysiometer or by measuring lactate concentration. Lactate concentration may be determined using an assay in which lactate is oxidized by lactate dehydrogenase to generate a product which interacts with a probe to produce a color (e.g., using a kit available from BioVision Inc., Milpitas, Calif., USA or Abcam Inc, Cambridge, Mass., USA) or by monitoring NADH production in a mixture that contains, in addition to lactic dehydrogenase and NAD⁺, hydrazine, and glycine buffer, pH 9.2. Absorbance due to formation of NADH can be detected at 340 nm using a spectrophotometer.

In some embodiments of any aspect herein, cells are cultured or measurement of OCR, ECAR, or cell survival or proliferation or any other parameter of interest is performed under conditions in which oxygen is present at levels equal to or greater than typical physiological levels. In some embodiments conditions such as those typically used in mammalian tissue culture, such as in a culture chamber controlled to have a gas composition with about a 5% CO₂ level and an oxygen level approximately that of atmospheric oxygen levels (21%) are used. In some embodiments conditions in which oxygen level is between about 1% and about 2%, about 2% and about 5%, about 5% to about 10%, or about 10% to about 20% are used.

### V. Combination Therapies and Agents of Use Therein

As described herein, Applicants found that MCT1 expression is the main determinant of tumor cell sensitivity to 3-BrPA and that MCT1 expression correlates with elevated glycolysis. Applicants also discovered that inhibition of glyceraldehyde-3-phosphate dehydrogenase (GAPDH) is the likely cause of the anti-glycolytic effects of 3-BrPA. In some aspects, the present disclosure provides a variety of compositions and methods for combination anti-cancer chemotherapy based at least in part on one or more of these discoveries. In some aspects, methods of identifying agents useful for inhibiting tumor cell survival or proliferation in combination with 3-BrPA thereof are provided. In some embodiments an agent enhances efficacy of 3-BrPA.

In some embodiments the efficacy of 3-BrPA is enhanced by concomitant treatment with an agent that inhibits glycolysis (a “glycolysis inhibitor”) so as to exploit the high glycolytic demand of tumors and the cancer-enriched expression of MCT1. In some aspects, the present disclosure provides a method of inhibiting survival or proliferation of a tumor cell that has increased MCT1 expression, the method comprising contacting the tumor cell with (a) 3-BrPA or an analog thereof; and (b) a glycolysis inhibitor. In some aspects, the present disclosure provides a method of treating cancer comprising treating a subject in need of treatment for a tumor that has increased MCT1 expression with 3-BrPA or an analog thereof and a glycolysis inhibitor.

A glycolysis inhibitor may inhibit any enzyme involved in glycolysis in various embodiments. In some embodiments a glycolysis inhibitor inhibits PFKFP3. In some embodiments a PFKFP3 inhibitor is 3-(3-pyridinyl)-1-(4-pyridinyl)-2-propen-1-one (3PO) (Clem et al., (2008), Mol. Cancer. Ther. 7: 110-120). In some embodiments a glycolysis inhibitor inhibits pyruvate kinase M2 (PKM2). In some embodiments a PKM2 inhibitor is a small molecule disclosed in PCT/US2007/017519 (published as WO/2008/019139) and/or disclosed in (9); in some embodiments a PKM2 inhibitor is shikonin, its enantiomeric isomer alkannin, or a shikonin or alkannin analog (Chen, J., et al., Oncogene. 2011; 30(42):4297-306); in some embodiments a PKM2 inhibitor is a peptide such as TLN-232/CAP-232. In some embodiments a glycolysis inhibitor inhibits pyruvate dehydrogenase (PDH). In some embodiments a glycolysis inhibitor inhibits at least one PDK, e.g., PDK1. In some embodiments an agent that inhibits at least one PDK is dichloroacetate (DCA). In some embodiments a glycolysis inhibitor inhibits LDH5. In some embodiments an LDH5 inhibitor is gossypol/AT-101 or an analog thereof, e.g., 3-dihydroxy-6-methyl-7-(phenylmethyl)-4-propylnaphthalene-1-carboxylicacid (FX11; Pubchem ID: 10498042) (Le, A., et al., (2010); PNAS, vol. 107 no. 5 2037-2042). Additional LDH5 inhibitors are disclosed in PCT/EP2010/006740 (published as WO2011054525), e.g., N-Hydroxy-2-carboxy-substituted indole compounds (Granchi, C., et al., 2011; J. Med. Chem. 54, 1599-1612. In some embodiments a glycolysis inhibitor inhibits carbonic anhydrase 9 (CA-9). In some embodiments a glycolysis inhibitor inhibits a glucose transporter, e.g., GLUT1 or GLUT3. In some embodiments a glycolysis inhibitor inhibits glucose uptake. In some embodiments an agent that inhibits glucose uptake is N-[4-chloro-3-(trifluoromethyl)phenyl]-3-oxobutanamide (fasentin) or an analog thereof (11). In some embodiments the second anti-cancer agent is 2-deoxyglucose.

In some embodiments, provided herein is a composition comprising 3-BrPA or an analog thereof and a second glycolysis inhibitor. In some embodiments a method of treating a subject in need of treatment for a tumor comprising tumor cells that have elevated MCT1 expression comprises administering a composition comprising 3-BrPA or an analog thereof and a second glycolysis inhibitor to the subject.

In some embodiments the glycolysis inhibitor is a GAPDH inhibitor. As discussed above, the present disclosure provides the recognition that inhibition of glyceraldehyde-3-phosphate dehydrogenase (GAPDH) is the likely cause of the anti-glycolytic effects of 3-BrPA; thus 3-BrPA exerts its main anti-glycolytic effects by acting as a GAPDH inhibitor. GAPDH (EC1.2.1.12; Human Gene ID; 2597; RefSeq Accession Numbers: NM_{—}002046.4 NP_{—}002037.2 (isoform 1); NM_{—}001256799.1→NP_{—}001243728.1 (isoform 2)), a key enzyme in glycolysis, catalyzes the oxidative phosphorylation of the triose glyceraldehyde 3-phosphate to form 1,3-diphosphoglycerate in the presence of NAD+ and inorganic phosphate. The term “GAPDH inhibitor” encompasses any agent that inhibits expression or activity of GAPDH. In some embodiments a GAPDH inhibitor comprises an RNAi agent, aptamer, peptide, or small molecule. In some aspects, the present disclosure provides a method of inhibiting survival or proliferation of tumor cells that have increased MCT1 expression, the method comprising: contacting tumor cells that have increased MCT1 expression with 3-BrPA or an analog thereof and a second GAPDH inhibitor. In some aspects, the present disclosure provides a method of treating cancer comprising: treating a subject in need of treatment for a tumor with 3-BrPA or an analog thereof and a second GAPDH inhibitor, wherein the tumor comprises tumor cells that have elevated MCT1 expression. Without wishing to be bound by any theory, it may be advantageous for any of a variety of reasons to use a combination of 3-BrPA or an analog thereof and a second GAPDH inhibitor to inhibit cancer cell survival or proliferation and/or to treat a subject in need of treatment for cancer. In some embodiments the combined effect of the two agents reduces the level of GAPDH activity below that achievable using either agent alone at doses tolerated by a subject. In some embodiments, the combination allows the use of lower doses of each agent than would be the case if either agent were used in the absence of the other without reduction in efficacy. In some embodiments, the combination reduces the survival or emergence of resistant tumor cells.

Pentalenolactone and koningic acid are antibiotics that potently inhibit GAPDH. The reactive groups present in these antibiotics are, respectively, an epoxide and an alpha-enone, which form covalent bonds with an active-site cysteine residue of the enzyme (Cane D E & Sohng J K. Archives of Biochemistry. 1989; 270(1): 50-61; Cane D E & Sohng J K. Biochemistry. 1994; 33(21):6524-30).

In some embodiments a GAPDH inhibitor is pentalenolactone or an analog thereof. Pentalenolactone is represented by the formula depicted below.

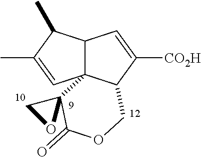

In some embodiments a pentalenolactone analog is tetrahydropentalenolactone. In some embodiments a pentalenolactone analog is an ester (e.g., a lower alkyl ester, such as methyl ester). In some embodiments the compound is provided as a salt, e.g., a benzylamine salt.

In some embodiments a GAPDH inhibitor is koningic acid (also called heptidilic acid) or an analog thereof. Koningic acid is represented by the formula depicted below.

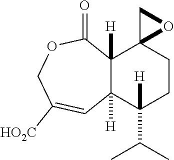

In some embodiments a koningic acid analog is a heptidylic acid halohydrin such as heptidylic acid chlorohydrin (depicted below).

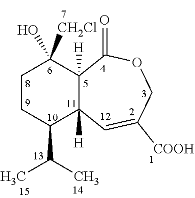

In some embodiments a GAPDH inhibitor is a glyceraldehyde 3-phosphate analog. A glyceraldehyde 3-phosphate analog may incorporate an epoxide, an alpha-enone, or another other reactive group that binds covalently to GAPDH, e.g., via reaction with a Cys-SH residue. Examples of such glyceraldehyde 3-phosphate analogs that act as inhibitors of GAPDH are disclosed in Willson, M., et al., Biochemistry. 1994; 33(1):214-20. Additional examples of GAPDH inhibitors are disclosed in, e.g., US Pat. Pub. No. 2006/0293325. For example, in some embodiments a GAPDH inhibitor is a compound of the following formula, wherein the substituents are as described in US Pat. Pub. No. 2006/0293325:

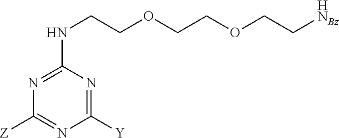

In some embodiments, provided herein is a composition comprising 3-BrPA or an analog thereof and a second GAPDH inhibitor. In some embodiments a method of treating a subject in need of treatment for a tumor comprising tumor cells that have elevated MCT1 expression comprises administering a composition comprising 3-BrPA or an analog thereof and a second GAPDH inhibitor to the subject.

In some aspects, the disclosure provides a method comprising (a) identifying a GAPDH inhibitor; and (b) contacting cells with the GAPDH inhibitor and 3-BrPA or an analog thereof. In some embodiments the method further comprises measuring survival and/or proliferation of the cells. In some embodiments the cells comprise tumor cells. In some embodiments the method comprises measuring the ability of the GAPDH inhibitor to inhibit tumor cell survival or proliferation. In some embodiments the method comprises measuring the ability of the combination of 3-BrPA and the GAPDH inhibitor to inhibit tumor cell survival or proliferation.

Any of a variety of methods can be used to identify a GAPDH inhibitor. Methods of assaying GAPDH activity are known in the art. In some embodiments GAPDH activity is measured by providing an assay composition comprising a GAPDH protein and assessing NADH production. NADH can be detected, e.g., spectrophotometrically by measuring absorbance at 340 nm or fluorimetrically by excitation at 340 nm and detecting emission at 450 nm. A GAPDH protein can be produced recombinantly or purified from cells that produce it naturally or from tissues in which it is found. Rabbit muscle tissue is a commonly used source of GAPDH protein. In some embodiments human GAPDH protein, e.g., recombinant human GAPDH protein, is used. In some embodiments a method of identifying a GAPDH inhibitor comprises: (a) providing a composition comprising GAPDH protein, NAD+, glyceraldehyde 3-phosphate, and a test agent; and (b) measuring production of NADH, wherein a reduction in production of NADH in the presence of the test agent as compared with its absence indicates that the compound is a GAPDH inhibitor. The assay composition may contain additional components such as a buffer substance (e.g., sodium or potassium phosphate), EDTA, a salt such as KCl, etc. In some embodiments, GAPDH protein is incubated in the assay buffer with a test agent but without at least one of the components required for the reaction (e.g., NAD+). The omitted component is added to start the reaction. Production of NADH is monitored. If NADH production is less than that which would be expected had the test agent not been present, the test agent is identified as a GAPDH inhibitor. In some embodiments GAPDH protein is incubated in the presence of the test agent for varying amounts of time prior to the start of the reaction. In some embodiments the test agent is added to the assay composition prior to addition of the GAPDH. Other methods of identifying a GAPDH inhibitor may also be used. For example, NADH may be detected indirectly, by coupling the production of NADH to a second reaction that produces or consumes a substance, and detecting the production or consumption of the substance. The effectiveness of an identified compound as a GAPDH inhibitor may be confirmed by repeating the assay or performing a second assay or suitable control assay.

In some embodiments the disclosure provides a method of testing a combination anti-cancer therapy comprising: (a) contacting one or more tumor cells with a GAPDH inhibitor and 3-BrPA; and (b) assessing survival or proliferation of the one or more tumor cells, wherein a decrease in survival or proliferation of the one or more tumor cells indicates that the combination has potential use as a combination therapy for treating tumors that have increased expression of MCT1. In some embodiments the method comprises comparing the survival or proliferation of the tumor cells exposed to both agents with a reference level. In some embodiments the reference level is the level of survival or proliferation that would be expected had the tumor cell been contacted with about the same concentration of 3-BrPA but in the absence of the GAPDH inhibitor or with about the same concentration of the GAPDH inhibitor but in the absence of 3-BrPA. In some embodiments the tumor cells comprise cells that have increased expression of MCT1. In some embodiments the method is performed using cells growing in culture. In some embodiments the method is performed by administering the agents to a test animal that serves as a tumor model. The GAPDH inhibitor may be a GAPDH inhibitor known in the art or the method may comprise first identifying a GAPDH inhibitor and then testing it in combination with 3-BrPA. A 3-BrPA analog may be used instead of 3-BrPA in certain embodiments.

In some aspects, provided herein is a method of inhibiting tumor cell survival or proliferation, the method comprising contacting a tumor cell that expresses MCT1 with 3-BrPA or an analog thereof and an MCT1 inhibitor. In some embodiments the tumor cell has increased MCT1 expression. In some aspects, provided herein is a method of treating a subject in need of treatment for a tumor having increased expression of MCT1, the method comprising treating the subject with 3-BrPA or an analog thereof and an MCT1 inhibitor. In some aspects, provided herein is a composition comprising 3-BrPA or an analog thereof and an MCT1 inhibitor. In some embodiments a method of treating a subject in need of treatment for a tumor having increased expression of MCT1 comprises treating the subject with a composition comprising (a) 3-BrPA or an analog thereof and (b) an MCT1 inhibitor.

As used herein, the term “MCT1 inhibitor” refers to an agent that inhibits MCT1 expression or activity. In some embodiments an MCT1 inhibitor comprises an RNAi agent, aptamer, peptide, or small molecule. In some embodiments the RNAi agent inhibits expression of MCT1 or BSG. Certain MCT1 inhibitors have been reported to show promise as anti-cancer agents. For example, the MCT1 inhibitor α-cyano-4-hydroxycinnamate (CHC) was reported to show potent antitumor affects alone or in combination with radiotherapy in mice, without exerting overt toxicity (Sonveaux et al., J Clin Invest. 2008; 118(12):3930-42; Vegran et al., 2011; Cancer Res. 71, 2550-2560). Without wishing to be bound by any theory, it may be advantageous to use a combination of 3-BrPA or an analog thereof and an MCT1 inhibitor. While it is expected that an MCT1 inhibitor would reduce sensitivity of cancer cells that express MCT1 to 3-BrPA, the presence of 3-BrPA would limit the ability of such cells to upregulate MCT1 and thereby acquire resistance to the MCT1 inhibitor. Tumor cells that upregulate MCT1 would exhibit increased sensitivity to 3-BrPA and, therefore, would be inhibited or eliminated by the combination therapy.

Various MCT1 inhibitors are described in Murray, C. M., et al. (2005) Nat. Chem. Biol. 1, 371-376. Guile, et al. (2006), Bioorg. Medicinal. Chem. Lett. 16, 2260-2265; PCT/SE2004/000052 (published as WO/2004/065394) and/or in PCT/GB2010/050096 (published as WO/2010/089580).

In some embodiments an MCT1 inhibitor is a compound of Formula III, wherein R¹, R², R³, Q, and Ar are as described in WO/2004/065394 or WO/2010/089580:

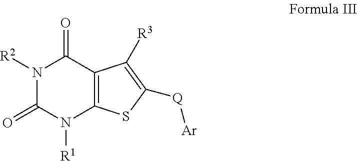

In some embodiments an MCT1 inhibitor is AR-C155858.

In some embodiments an MCT1 inhibitor is AR-C117977.

In some embodiments an MCT1 inhibitor is the compound known as AR-C155858 (depicted below).

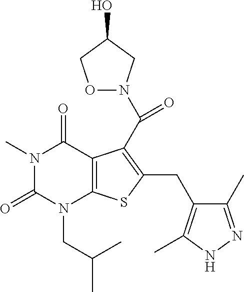

In some embodiments an MCT1 inhibitor is the compound known as AZD3965.

In some embodiments the MCT inhibitor is 6-[[3,5-dimethyl-1-(2-pyridinyl)-1/f-pyrazol-4-yl]methyl]-5-[[(4S)-4-hydroxy-4-methyl-2-isoxazolidinyl]carbonyl]-3-methyl-1-(1-methylethyl)thieno[2,3-d]pyrimidine-2,4-(1/f, 3H)-dione; 5-[[(45)-4-hydroxy-4-methyl-2-isoxazolidinyl]carbonyl]-3-methyl-1-(1-methylethyl)-6-[[5-methyl-3-(trifluoromethyl)-1H-pyrazol-4-yl]methyl]-thieno[2,3-(i]pyrimidine-2,4(1H,3H)-dione; or (45)-4-methyl-2-[[1,2,3,4-tetrahydro-3-methyl-1-(1-methylethyl)-6-[[5-methyl-1-(2-pyrimidinyl)-3-(trifluoromethyl)-1H-pyrazol-4-yl]methyl]-2,4-dioxothieno[2,3-<i]pyrimidin-5-yl]carbonyl]-4-isoxazolidinol or a pharmaceutically acceptable salt of any of these.

In some embodiments an MCT1 inhibitor is α-cyano-4-hydroxycinnamate or an analog thereof. The structure of α-cyano-4-hydroxycinnamate is depicted below:

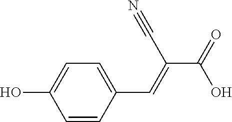 [SMILES: N#C/C(=C\c1ccc(O)cc1)C(=O)O]

In some embodiments an α-cyano-4-hydroxycinnamate analog comprises a substituent on at least one position on the phenyl ring of the above structure.

In some embodiments an MCT1 inhibitor selectively binds to and/or inhibits MCT1 as compared with MCT2, MCT3, and/or MCT4. In some embodiments the K_{d} for binding to MCT1 is at least 5-, 10-, 20-, 50-, 10², 10³, or 10⁴-fold lower than for binding to MCT2, MCT3, and/or MCT4. Methods useful for assessing binding affinity will be apparent to those of ordinary skill in the art. In some embodiments binding affinity is measured by a competition assay or by surface plasmon resonance, e.g., Biacore assay. In some embodiments a ligand-binding assay, e.g., using scintillation proximity assay (SPA) technology, is used, such as that described in Guile, et al., supra. In some embodiments the IC₅₀ of an MCT1 inhibitor for inhibiting MCT1 may be at least 5-, 10-, 20-, 50-, 10², 10³, or 10⁴ lower than its IC₅₀ for inhibiting MCT2, MCT3, and/or MCT4. In some embodiments inhibition refers to causing a reduction in transport of a substrate. In some embodiments the substrate is a monocarboxylate, e.g., lactate or pyruvate. Exemplary methods useful for assessing transport are described in the Examples.

In some embodiments a plurality of test agents are screened to identify GAPDH or MCT1 modulators, e.g., GAPDH or MCT1 inhibitors. Test agents and screening approaches such as those described in Section III may be used. The particular assay to be used in the screen may be an enzymatic activity assay (for GAPDH) or a binding assay.

It will be understood that screens or assays to identify or test modulators of a particular polypeptide (e.g., a GAPDH or MCT1 polypeptide) may make use of variants of the particular polypeptide. For example, functional variants may be used. In some embodiments a functional variant may comprise a heterologous polypeptide portion, such as an epitope tag or fluorescent protein, which may facilitate detection or isolation.

In some embodiments a computer-aided computational approach sometimes referred to as “virtual screening” is used in the identification of candidate GAPDH or MCT1 modulators, e.g., candidate GAPDH or MCT1 inhibitors. Structures of compounds may be screened for ability to bind to a region (e.g., a “pocket”) of a target molecule (GAPDH or MCT1) that is accessible to the compound. The region may be a known or potential active site or any region accessible to the compound, e.g., a concave region on the surface or a cleft or the pore of a transporter. A variety of docking and pharmacophore-based algorithms are known in the art, and computer programs implementing such algorithms are available. Commonly used programs include Gold, Dock, Glide, FlexX, Fred, and LigandFit (including the most recent releases thereof). See, e.g., Ghosh, S., et al., Current Opinion in Chemical Biology, 10(3): 194-2-2, 2006; McInnes C., Current Opinion in Chemical Biology; 11(5): 494-502, 2007, and references in either of the foregoing articles, which are incorporated herein by reference. In some embodiments a virtual screening algorithm may involve two major phases: searching (also called “docking”) and scoring. During the first phase, the program automatically generates a set of candidate complexes of two molecules (test compound and target molecule) and determines the energy of interaction of the candidate complexes. The scoring phase assigns scores to the candidate complexes and selects a structure that displays favorable interactions based at least in part on the energy. To perform virtual screening, this process may be repeated with a large number of test compounds to identify those that, for example, display the most favorable interactions with the target. In some embodiments, low-energy binding modes of a small molecule within an active site or possible active site are identified. Variations may include the use of rigid or flexible docking algorithms and/or including the potential binding of water molecules.

Numerous small molecule structures are available and can be used for virtual screening. A collection of compound structures may sometimes referred to as a “virtual library”. For example, ZINC is a publicly available database containing structures of millions of commercially available compounds that can be used for virtual screening (http://zinc.docking.org/; Shoichet, J. Chem. Inf. Model., 45(1):177-82, 2005). A database containing about 250,000 small molecule structures is available on the National Cancer Institute (U.S.) website (at http://129.43.27.140/ncidb2/). In some embodiments multiple small molecules may be screened, e.g., up to 50,000; 100,000; 250,000; 500,000, or up to 1 million, 2 million, 5 million, 10 million, or more. Compounds can be scored and, optionally, ranked by their potential to bind to a target. Compounds identified in virtual screens can be tested in cell-free or cell-based assays or in animal models to confirm their ability to inhibit activity of GAPDH or MCT1 and/or to assess their effect on survival or proliferation of tumor cells in vitro or in vivo.

Computational approaches can be used to predict one or more physico-chemical, pharmacokinetic and/or pharmacodynamic properties of compounds identified in physical or virtual screens. For example, absorption, distribution, metabolism, and excretion (ADME) parameters can be predicted. Such information can be used, e.g., to select hits for further testing or modification. For example, small molecules having characteristics typical of “drug-like” molecules can be selected and/or small molecules having one or more undesired characteristics can be avoided.

In some embodiments any of the method may comprise testing 3-BrPA or an analog thereof and a second agent, e.g., a GAPDH inhibitor or MCT inhibitor, together in a tumor model. A tumor model may comprise cultured tumor cells or may be an in vivo model. Examples of tumor models are described herein.

In some embodiments 3-BrPA or an analog thereof is used to treat a subject in need of treatment for a tumor having increased expression of MCT1 in combination with any one or more additional anti-cancer therapeutic modalities (e.g., chemotherapeutic drugs, surgery, radiotherapy (e.g., γ-radiation, neutron beam radiotherapy, electron beam radiotherapy, proton therapy, brachytherapy, and systemic radioactive isotopes), endocrine therapy, immunotherapy, biologic response modifiers (e.g., interferons, interleukins), hyperthermia (e.g., radiofrequency ablation or other methods of delivering heat such as using lasers, high intensity focused ultrasound or microwaves), cryotherapy, etc.) or combinations thereof, useful for treating a subject in need of treatment for a tumor. Agents used in combination may be administered in the same composition or separately in various embodiments. When they are administered separately, two or more agents may be given simultaneously or sequentially (in any order). If administered separately, the time interval between administration of the agents can vary. Agents or non-pharmacological therapies used in combination can be administered or used in any temporal relation to each other such that they produce a beneficial effect in at least some subjects. In some embodiments a beneficial effect produced by a combination is at least as great as, or greater than, that which would be achieved by each therapy individually. In some embodiments, administration of first and second agents is performed such that (i) a dose of the second agent is administered before more than 90% of the most recently administered dose of the first agent has been metabolized to an inactive form or excreted from the body; or (ii) doses of the first and second agents are administered at least once within 8 weeks of each other (e.g., within 1, 2, 4, or 7 days, or within 2, 3, 4, 5, 6, 7, or 8 weeks of each other); (iii) the therapies are administered at least once during overlapping time periods (e.g., by continuous or intermittent infusion); or (iv) any combination of the foregoing. In some embodiments agents may be administered individually at substantially the same time (e.g., within less than 1, 2, 5, or 10 minutes of one another). In some embodiments agents may be administered individually within less than 3 hours, e.g., less than 1 hour. In some embodiments agents may be administered by the same route of administration. In some embodiments agents may be administered by different routes of administration. It will be understood that any of the afore-mentioned time frames pertaining to combination therapy may apply to agents and/or to non-pharmacological therapies such as hyperthermia, externally administered radiotherapy, etc.

A “regimen” or “treatment protocol” refers to a selection of one or more agent(s), dose level(s), and optionally other aspects(s) that describe the manner in which therapy is administered to a subject, such as dosing interval, route of administration, rate and duration of a bolus administration or infusion, appropriate parameters for administering radiation, etc. Many cancer chemotherapy regimens include combinations of drugs that have different cytotoxic or cytostatic mechanisms and/or that typically result in different dose-limiting adverse effects. For example, an agent that acts on DNA (e.g., alkylating agent) and an anti-microtubule agent are a common combination found in many chemotherapy regimens.

For purposes herein a regimen that has been tested in a clinical trial, e.g., a regimen that has been shown to be acceptable in terms of safety and, in some embodiments, showing at least some evidence of efficacy, will be referred to as a “standard regimen” and an agent used in such a regimen may be referred to as a “standard chemotherapy agent”. In some embodiments a standard regimen or standard chemotherapy agent is a regimen or chemotherapy agent that is used in clinical practice in oncology. In some embodiments pharmaceutical agents used in a standard regimen are all approved drugs. See, e.g., DeVita, supra for examples of standard regimens. It will be understood that different standard regiments may be selected as appropriate based on factors such as tumor type, tumor grade, tumor stage, concomitant illnesses, concomitant illnesses, general condition of the patient, etc.

In some embodiments 3-BrPA and, in some embodiments, one or more additional glycolysis inhibitors, is added to a standard regimen or substituted for one or more of the agents typically used in a standard regimen. Non-limiting examples of cancer chemotherapeutic agents that may be used include, e.g., alkylating and alkylating-like agents such as nitrogen mustards (e.g., chlorambucil, chlormethine, cyclophosphamide, ifosfamide, and melphalan), nitrosoureas (e.g., carmustine, fotemustine, lomustine, streptozocin); platinum agents (e.g., alkylating-like agents such as carboplatin, cisplatin, oxaliplatin, BBR3464, satraplatin), busulfan, dacarbazine, procarbazine, temozolomide, thioTEPA, treosulfan, and uramustine; antimetabolites such as folic acids (e.g., aminopterin, methotrexate, pemetrexed, raltitrexed); purines such as cladribine, clofarabine, fludarabine, mercaptopurine, pentostatin, thioguanine; pyrimidines such as capecitabine, cytarabine, fluorouracil, floxuridine, gemcitabine; spindle poisons/mitotic inhibitors such as taxanes (e.g., docetaxel, paclitaxel), vincas (e.g., vinblastine, vincristine, vindesine, and vinorelbine), epothilones; cytotoxic/anti-tumor antibiotics such anthracyclines (e.g., daunorubicin, doxorubicin, epirubicin, idarubicin, mitoxantrone, pixantrone, and valrubicin), compounds naturally produced by various species of Streptomyces (e.g., actinomycin, bleomycin, mitomycin, plicamycin) and hydroxyurea; topoisomerase inhibitors such as camptotheca (e.g., camptothecin, topotecan, irinotecan) and podophyllums (e.g., etoposide, teniposide); monoclonal antibodies for cancer therapy such as anti-receptor tyrosine kinases (e.g., cetuximab, panitumumab, trastuzumab), anti-CD20 (e.g., rituximab and tositumomab), and others for example alemtuzumab, aevacizumab, gemtuzumab; photosensitizers such as aminolevulinic acid, methyl aminolevulinate, porfimer sodium, and verteporfin; tyrosine and/or serine/threonine kinase inhibitors, e.g., inhibitors of Abl, Kit, insulin receptor family member(s), VEGF receptor family member(s), EGF receptor family member(s), PDGF receptor family member(s), FGF receptor family member(s), mTOR, Raf kinase family, phosphatidyl inositol (PI) kinases such as PI3 kinase, PI kinase-like kinase family members, cyclin dependent kinase (CDK) family members, Aurora kinase family members (e.g., kinase inhibitors that are on the market or have shown efficacy in at least one phase III trial in tumors, such as cediranib, crizotinib, dasatinib, erlotinib, gefitinib, imatinib, lapatinib, nilotinib, sorafenib, sunitinib, vandetanib), growth factor receptor antagonists, and others such as retinoids (e.g., alitretinoin and tretinoin), altretamine, amsacrine, anagrelide, arsenic trioxide, asparaginase (e.g., pegasparagase), bexarotene, bortezomib, denileukin diftitox, estramustine, ixabepilone, masoprocol, mitotane, and testolactone, Hsp90 inhibitors, proteasome inhibitors (e.g., bortezomib), angiogenesis inhibitors, e.g., anti-vascular endothelial growth factor agents such as bevacizumab (Avastin) or VEGF receptor antagonists, matrix metalloproteinase inhibitors, various pro-apoptotic agents (e.g., apoptosis inducers), Ras inhibitors, anti-inflammatory agents, cancer vaccines, or other immunomodulating therapies, RNAi agents targeted to oncogenes, etc. It will be understood that the preceding classification is non-limiting. A number of anti-tumor agents have multiple activities or mechanisms of action and could be classified in multiple categories or classes or have additional mechanisms of action or targets.

In some embodiments, 3-BrPA is used to treat liver cancer in combination with one or more additional anti-cancer agents, wherein the one or more additional anti-cancer agents are approved or used in the art for treatment of liver cancer. In some embodiments the additional anticancer agent is a kinase inhibitor. In some embodiments the kinase inhibitor inhibits one or more tyrosine kinases. In some embodiments the kinase inhibitor is sorafenib, a small molecular inhibitor of several tyrosine protein kinases (e.g., VEGFR and PDGFR) and Raf kinases (Wilhelm S M, et al., Molecular Cancer Therapeutics (2008); 7 (10): 3129-40).

### VI. Pharmaceutical Compositions and Methods of Treatment

Agents and compositions disclosed herein or identified as disclosed herein may be administered to a subject, e.g., a subject in need of treatment of cancer, by any suitable route such as by intravenous, intraarterial, oral, intranasal, subcutaneous, intramuscular, intraosseus, intrasternal, intraperitoneal, intrathecal, intratracheal, intraocular, sublingual, vaginal, rectal, dermal, or pulmonary administration. Administration of a compound of composition may thus comprise introducing a compound or composition into or onto the body by any suitable route. Depending upon the type of condition (e.g., cancer) to be treated, agents may, for example, be introduced into the vascular system, inhaled, ingested, etc. Thus, a variety of administration modes, or routes, are available. The particular mode selected will, in various embodiments, generally depend on one or more factors such as the particular cancer being treated, the dosage required for therapeutic efficacy, and agents (if any) used in combination. The methods, generally speaking, may be practiced using any mode of administration that is medically or veterinarily acceptable, meaning any mode that produces acceptable levels of efficacy without causing clinically unacceptable (e.g., medically or veterinarily unacceptable) adverse effects. The term “parenteral” includes intravenous, intraarterial, intramuscular, intraperitoneal, subcutaneous, intraosseus, and intrasternal injection, or infusion techniques. In some embodiments a method comprises dispensing a compound or composition for administration to a subject as described herein. In some embodiments administration comprises self-administration.

It will be understood that in some embodiments administration of an agent or composition may be performed for one or more purposes in addition to or instead of for treatment purposes. For example, in some embodiments a detection reagent is administered for purposes of in vivo detection of MCT1 expression. In some embodiments an agent or composition is administered for diagnosis or monitoring or for testing the agent or composition.

In some embodiments a route or location of administration is selected based at least in part on the location of a tumor. For example, an agent or composition may be administered locally, e.g., to or near a tissue or organ harboring or suspected of harboring a tumor or from which a tumor has been removed. Local delivery may increase the anti-tumor effect by locally increasing the concentration of the agent at the tumor site as compared with the concentration that would be achieved using other delivery approaches, may reduce metabolism or clearance as compared with systemic administration, or may reduce the incidence or severity of side effects as compared with systemic administration. In some embodiments administration near a tissue or organ harboring or suspected of harboring a tumor or from which a tumor has been removed comprises administration within up to 5 cm, 10 cm, 15 cm, 20 cm, or 25 cm from the edge or margin of the tumor or organ.

In some embodiments, a method comprises administering 3-BrPA or an analog thereof locally by administering it directly into the arterial blood supply of a tumor in a subject. The agent or composition may be administered into the artery using standard methods known in the art. In some embodiments the agent or composition is administered using a catheter. Insertion of the catheter may be guided or observed by imaging, e.g., fluoroscopy, or other suitable methods known in the art. In certain embodiments intraarterial administration is via continuous intraarterial infusion, which may deliver the agent at a controlled rate over a specified time period. The rate and duration of infusion may be controlled through the use of an external pump. Implantable pump systems suitable for administration of chemotherapeutic agents are available from a variety of manufacturers such as Bard Access Systems (Salt Lake City, Utah, USA). In some embodiments a continuous intraarterial infusion may be administered to a subject for between about 30 minutes to about 4 hours. In certain embodiments, a continuous intraarterial infusion is administered to a subject for at least about 1 hour to about 3 hours. In certain embodiments, the continuous intraarterial infusion is administered to the subject for about 1 hour. In some embodiments a dose of the agent or composition is administered via a bolus, i.e., a rapid push of the dose into the artery over a few minutes (e.g., between 2-10 minutes).

In some embodiments a catheter is inserted for a single administration of the agent or composition each time chemotherapy is administered. Such an approach may be suitable in situations in which the time required for an intraarterial injection is short and where administration is performed at a relatively low frequency. In some embodiments a reservoir system is embedded under the skin for continuous infusion or repetitive administration. Such an approach may be suitable when a long period of time is required for each administration, or when the administration is required at a relatively high frequency. The selection of an appropriate delivery method will be at the discretion of the ordinary skilled practitioner.

In the case of liver cancer, hepatic arterial infusion chemotherapy (HAIL) may be performed. Methods for implanting an indwelling a reservoir system for treatment of liver cancer include (i) the gastroduodenal artery coil method, by which the tip of a side-hole catheter is fixed and indwelled to the gastroduodenal artery via the femoral artery or the left subclavian artery; and (ii) the hepatic periphery fixation method, by which the catheter is fixed to the peripheral of the hepatic artery.

In some embodiments an anticancer agent, e.g., 3-BrPA, is administered in a composition comprising iodized oil (e.g., Lipiodol® also called Ethiodol). Iodized oil has been shown to act as a carrier of various chemotherapeutic agents, which are released slowly from the mixture. Lipiodol contains iodine combined with ethyl esters of fatty acids of poppyseed oil. Iodized oil has been found to remain selectively in the neovasculature and extravascular spaces of liver tumors when injected into the hepatic artery. In some embodiments radioactively labeled Lipiodol is used (e.g., containing iodine-131 or rhenium-188 labeled lipiodol. In some embodiments iodized oil is not used.

In some embodiments embolization is performed. Embolization refers to selective occlusion of blood vessels by purposely introducing embolic agents. In the context of tumor treatment, the purpose of embolization is to reduce or prevent blood flow to the tumor, which results in death of at least some of the cells in the tumor. Vascular occlusion may be accomplished using a variety of different embolic agents. Embolic agents may be liquid, e.g., viscous liquids such as Lipiodol, semi-solid, or solid. Examples of embolic agents include, e.g., gelatin sponge (e.g., Gelfoam), starch microspheres, polyvinyl alcohol beads, collagen particles, degradable starch microspheres (e.g., EmboCept®, PharmaCept GmbH, Berlin-Schöneberg, Germany), other polymer-containing microspheres (e.g., Embozene® (CeloNova BioSciences, Atlanta, Ga., USA) or Embosphere® (Biosphere Medical, Rockland, Mass., USA). Embozene is Embosphere is polymeric microsphere made of trisacryl cross-linked with gelatin. In some embodiments microspheres with diameters ranging from about 40 μm to about 1200 μm are used, e.g., 40-120, 100-300, 500-700, 700-900, or 900-1200 μm. Embolization is usually performed under imaging guidance, e.g., by an interventional radiologist.

In some embodiments chemoembolization, also called transcatheter arterial chemoemobolization (TACE), is performed. In some embodiments chemoembolization, also called transcatheter arterial chemoemobolization (TACE), is performed. Chemoembolization is a procedure in which anticancer drug(s) are administered directly into blood vessel(s) supplying a tumor, with concurrent or subsequent blockage of the feeding vessel by occlusive agents that are injected through the delivery catheter. Sometimes, the anticancer drug(s) are provided at least in part by small drug-eluting beads that are injected into an artery that feeds the tumor. The beads block blood flow to the tumor as they release the drug. Examples of such beads include, e.g., DC Bead, HepaSphere and irinotecan-eluting beads. Chemoembolization agents and procedures for use in cancer treatment, with a focus on treatment of liver cancer, are reviewed in Tam, K Y, et al., European Journal of Pharmaceutical Sciences (2011); Vol. 44, Issues 1-2, pp. 1-10.

In some embodiments radioembolization is performed in combination with administration of 3-BrPA to treat liver cancer. Radioembolization combines delivery of internal radiation to the tumor with concomitant embolization. Radioembolization may be performed by administering yttrium 90-containing microspheres or holmium-166-loaded poly(L-lactic acid) microspheres to a tumor's feeding arter(ies).

In some embodiments chemical ablation, e.g., by percutaneous ethanol injection or percutaneous acetic acid injection into one or more tumor(s), or physical ablation, e.g., cryoablation or hyperthermic therapy, may be combined with administration of 3-BrPA to treat liver cancer.

In some embodiments, 3-BrPA or an analog thereof is administered prior to embolization, TACE (using standard chemotherapeutic agents) radioembolization, or RFA.

In some embodiments 3-BrPA or an analog thereof is administered following embolization, TACE (using standard chemotherapeutic agents), radioembolization, or RFA. Administration of 3-BrPA or an analog thereof may help eliminate residual tumor cells that have not been killed by the embolization or ablation procedure.

In some embodiments 3-BrPA or an analog thereof is administered without performing embolization or chemical or physical ablation of tumor.

In some embodiments 3-BrPA or an analog thereof is administered using a dose, dosing regimen, or formulation described in PCT/US2007/087740 (published as WO/2008/076964).

In some embodiments treating a subject in need of treatment for a tumor comprises administering one or more agents that reduce one or more side effects resulting from treatment of the tumor. For example, the one or more agents may control nausea or promote elimination or detoxification of substances released as a result of tumor lysis.

In some embodiments, inhaled medications are of use. Such administration allows direct delivery to the lung, e.g., for treatment of lung cancer, although it could be used to achieve systemic delivery in certain embodiments. In some embodiments, intrathecal administration may be used, e.g., in a subject with a tumor of the central nervous system, e.g., a brain tumor.

In some embodiments an agent or composition is administered prior to, during, and/or following ablation, radiation, or surgical removal. Treatment prior to ablation, radiation, or surgery may be performed at least in part to reduce the size of the tumor and render it more amenable to ablation, radiation, or surgical therapy. Treatment during or after ablation, radiation, or surgery may be performed at least in part to eliminate residual tumor cells and/or to reduce the likelihood of recurrence.

Suitable preparations, e.g., substantially pure preparations, of an active agent (e.g., 3-BrPA or an analog thereof) may be combined with one or more pharmaceutically acceptable carriers or excipients, etc., to produce an appropriate pharmaceutical composition. In some embodiments, a pharmaceutically acceptable compositions for administration to a subject comprises (i) 3-BrPA or an analog thereof; and (ii) a pharmaceutically acceptable carrier or excipient. The term “pharmaceutically acceptable carrier or excipient” refers to a carrier (which term encompasses carriers, media, diluents, solvents, vehicles, etc.) or excipient which does not significantly interfere with the biological activity or effectiveness of the active ingredient(s) of a composition and which is not excessively toxic to the host at the concentrations at which it is used or administered. Other pharmaceutically acceptable ingredients can be present in the composition as well. Suitable substances and their use for the formulation of pharmaceutically active compounds is well-known in the art (see, for example, “Remington's Pharmaceutical Sciences”, E. W. Martin, 19th Ed., 1995, Mack Publishing Co.: Easton, Pa., and more recent editions or versions thereof, such as Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types).

A pharmaceutical composition is typically formulated to be compatible with its intended route of administration. For example, preparations for parenteral administration include sterile aqueous or non-aqueous solutions, suspensions, and emulsions. Aqueous carriers include water, alcoholic/aqueous solutions, emulsions or suspensions, including saline and buffered media, e.g., sodium chloride solution, Ringer's dextrose, dextrose and sodium chloride, lactated Ringer's. Examples of non-aqueous solvents are propylene glycol, polyethylene glycol, vegetable oils such as olive oil, and injectable organic esters such as ethyl oleate, fixed oils, polyethylene glycols, glycerine, propylene glycol or other synthetic solvents; preservatives, e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. pH can be adjusted with acids or bases, such as hydrochloric acid or sodium hydroxide. Such parenteral preparations can be enclosed in ampoules, disposable syringes or multiple dose vials made of glass or plastic. Pharmaceutical compositions and agents for use in such compositions may be manufactured under conditions that meet standards or criteria prescribed by a regulatory agency such as the US FDA (or similar agency in another jurisdiction) having authority over the manufacturing, sale, and/or use of therapeutic agents. For example, such compositions and agents may be manufactured according to Good Manufacturing Practices (GMP) and/or subjected to quality control procedures appropriate for pharmaceutical agents to be administered to humans.

For oral administration, agents can be formulated by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Suitable excipients for oral dosage forms are, e.g., fillers such as sugars, including) lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art.

Formulations for oral delivery may incorporate agents to improve stability in the gastrointestinal tract and/or to enhance absorption.

For administration by inhalation, pharmaceutical compositions may be delivered in the form of an aerosol spray from a pressured container or dispenser which contains a suitable propellant, e.g., a gas such as carbon dioxide, a fluorocarbon, or a nebulizer. Liquid or dry aerosol (e.g., dry powders, large porous particles, etc.) can be used. The disclosure contemplates delivery of compositions using a nasal spray or other forms of nasal administration. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers.

For topical applications, pharmaceutical compositions may be formulated in a suitable ointment, lotion, gel, or cream containing the active components suspended or dissolved in one or more pharmaceutically acceptable carriers suitable for use in such composition.

For local delivery to the eye, pharmaceutical compositions may be formulated as solutions or micronized suspensions in isotonic, pH adjusted sterile saline, e.g., for use in eye drops, or in an ointment. In some embodiments intraocular administration is used. Routes of intraocular administration include, e.g., intravitreal injection, retrobulbar injection, peribulbar injection, subretinal, sub-Tenon injection, and subconjunctival injection.

Pharmaceutical compositions may be formulated for transmucosal or transdermal delivery. For transmucosal or transdermal administration, penetrants appropriate to the barrier to be permeated may be used in the formulation. Such penetrants are generally known in the art. Pharmaceutical compositions may be formulated as suppositories (e.g., with conventional suppository bases such as cocoa butter and other glycerides) or as retention enemas for rectal delivery.

In some embodiments, a pharmaceutical composition includes one or more agents intended to protect the active agent(s) against rapid elimination from the body, such as a controlled release formulation, implants (e.g., macroscopic implants such as discs, wafers, etc.), microencapsulated delivery system, etc. Compounds may be encapsulated or incorporated into particles, e.g., microparticles or nanoparticles. Biocompatible polymers, e.g., biodegradable biocompatible polymers, can be used, e.g., in the controlled release formulations, implants, or particles. A polymer may be a naturally occurring or artificial polymer. Depending on the particular polymer, it may be synthesized or obtained from naturally occurring sources. An agent may be released from a polymer by diffusion, degradation or erosion of the polymer matrix, or combinations thereof. A polymer or combination of polymers, or delivery format (e.g., particles, macroscopic implant) may be selected based at least in part on the time period over which release of an agent is desired. A time period may range, e.g., from a few hours (e.g., 3-6 hours) to a year or more. In some embodiments a time period ranges from 1-2 weeks up to 3-6 months, or between 6-12 months. After such time period release of the agent may be undetectable or may be below therapeutically useful or desired levels. A polymer may be a homopolymer, copolymer (including block copolymers), straight, branched-chain, or cross-linked. Various polymers of use in drug delivery are described in Jones, D., Pharmaceutical Applications of Polymers for Drug Delivery, ISBN 1-85957-479-3, ChemTec Publishing, 2004. Useful polymers include, but are not limited to, poly-lactic acid (PLA), poly-glycolic acid (PGA), poly-lactide-co-glycolide (PLGA), poly(phosphazine), poly (phosphate ester), polycaprolactones, polyanhydrides, ethylene vinyl acetate, polyorthoesters, polyethers, and poly (beta amino esters). Other polymers useful in various embodiments include polyamides, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, poly(butyric acid), poly(valeric acid), and poly(lactide-cocaprolactone). Peptides, polypeptides, proteins such as collagen or albumin, polysaccharides such as sucrose, chitosan, dextran, alginate, hyaluronic acid (or derivatives of any of these) and dendrimers are of use in certain embodiments. Methods for preparation of such will be apparent to those skilled in the art. Additional polymers include cellulose derivatives such as, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxymethylcellulose, carboxylethyl cellulose, cellulose triacetate, cellulose sulphate sodium salt, polycarbamates or polyureas, cross-linked poly(vinyl acetate) and the like, ethylene-vinyl ester copolymers such as ethylene-vinyl acetate (EVA) copolymer, ethylene-vinyl hexanoate copolymer, ethylene-vinyl propionate copolymer, ethylene-vinyl butyrate copolymer, ethylene-vinyl pentantoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer, and ethylene-vinyl benzoate copolymer, or mixtures thereof. Chemical derivatives of the afore-mentioned polymers, e.g., substitutions, additions of chemical groups, for example, alkyl, alkylene, hydroxylations, oxidations, and other modifications routinely made by those skilled in the art can be used. A particle, implant, or formulation may be composed of a single polymer or multiple polymers. A particle or implant may be homogeneous or non-homogeneous in composition. In some embodiments a particle comprises a core and at least one shell or coating layer, wherein, in some embodiments, the composition of the core differs from that of the shell or coating layer. A therapeutic agent or label may be physically associated with a particle, formulation, or implant in a variety of different ways. For example, agents may be encapsulated, attached to a surface, dispersed homogeneously or nonhomogeneously in a matrix, etc. Methods for preparation of such formulations, implants, or particles will be apparent to those skilled in the art. Liposomes or other lipid-containing particles can be used as pharmaceutically acceptable carriers in certain embodiments. In some embodiments a controlled release formulation, implant, or particles may be introduced or positioned within a tumor, near a tumor or its blood supply, in or near a region from which a tumor was removed, at or near a site of known or potential metastasis (e.g., a site to which a tumor is prone to metastasize), etc. Microparticles and nanoparticles can have a range of dimensions. In some embodiments a microparticle has a diameter between 100 nm and 100 μm. In some embodiments a microparticle has a diameter between 100 nm and 1 μm, between 1 μm and 20 μm, or between 1 μm and 10 μm. In some embodiments a microparticle has a diameter between 100 nm and 250 nm, between 250 nm and 500 nm, between 500 nm and 750 nm, or between 750 nm and 1 μm. In some embodiments a nanoparticle has a diameter between 10 nm and 100 nm, e.g., between 10 nm and 20 nm, between 20 nm and 50 nm, or between 50 nm and 100 nm. In some embodiments particles are substantially uniform in size or shape. In some embodiments particles are substantially spherical. In some embodiments a particle population has an average diameter falling within any of the afore-mentioned size ranges. In some embodiments a particle population consists of between about 20% and about 100% particles falling within any of the afore-mentioned size ranges or a subrange thereof, e.g. about 40%, 40%, 50%, 60%, 70%, 80%, 90%, 95%, etc. In the case of non-spherical particles, the longest straight dimension between two points on the surface of the particle rather than the diameter may be used as a measure of particle size. Such dimension may have any of the length ranges mentioned above. In some embodiments a particle comprises a detectable label or detection reagent or has a detectable label or detection reagent attached thereto. In some embodiments a particle is magnetic, e.g., to facilitate removal or separation of the particle from a composition that comprises the particle and one or more additional components.

Forms of polymeric matrix that may contain and/or be used to deliver an agent include films, coatings, gels (e.g., hydrogels), which may be implanted or applied to an implant or indwelling device such as a stent or catheter.

In general, the size, shape, and/or composition of a polymeric material, matrix, or formulation may be appropriately selected to result in release in therapeutically useful amounts over a useful time period, in the tissue into the polymeric material, matrix, or formulation is implanted or administered.

In some embodiments, a pharmaceutically acceptable salt, ester, salt of such ester, active metabolite, prodrug, or any adduct or derivative of 3-BrPA or an analog thereof which upon administration to a subject in need thereof is capable of providing the compound, directly or indirectly, is used. In some embodiments a pharmaceutically acceptable salt, ester, salt of such ester, active metabolite, prodrug, or adduct or derivative of 3-BrPA or an analog thereof may be formulated and, in general, used for the same purpose(s) as 3-BrPA.

The term “pharmaceutically acceptable salt” refers to those salts which are, within the scope of sound medical judgment, suitable for use in contact with the tissues of humans and/or lower animals without undue toxicity, irritation, allergic response and the like, and which are commensurate with a reasonable benefit/risk ratio. A wide variety of appropriate pharmaceutically acceptable salts are well known in the art. Pharmaceutically acceptable salts include, but are not limited to, those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. In some embodiments cases, a compound may contain one or more acidic functional groups and, thus, be capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary, tertiary, or quaternary amine. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Representative organic amines useful for the formation of base addition salts include ethylamine, diethylamine, ethylenediamine, ethanolamine, diethanolamine, piperazine and the like.

A therapeutically effective dose of an active agent in a pharmaceutical composition may be within a range of about 1 μg/kg to about 500 mg/kg body weight, about 0.001 mg/kg to about 100 mg/kg, about 0.001 mg/kg to about 10 mg/kg, about 0.01 mg/kg to about 25 mg/kg, about 0.1 mg/kg to about 20 mg/kg body weight, about 1 mg/kg to about 10 mg/kg, about 1 mg/kg to about 3 mg/kg, about 3 mg/kg to about 5 mg/kg, about 5 mg/kg to about 10 mg/kg. In some embodiments doses of agents described herein may range, e.g., from about 10 μg to about 10,000 mg, e.g., from about 100 μg to about 5,000 mg, e.g., from about 0.1 mg to about 1000 mg once or more per day, week, month, or other time interval, in various embodiments. In some embodiments a dose is expressed in terms of mg/m² body surface area. Body surface area may be estimated using standard methods. In some embodiments a single dose is administered while in other embodiments multiple doses are administered. Those of ordinary skill in the art will appreciate that appropriate doses in any particular circumstance depend upon the potency of the agent(s) utilized, and may optionally be tailored to the particular recipient. The specific dose level for a subject may depend upon a variety of factors including the activity of the specific agent(s) employed, severity of the disease or disorder, the age, body weight, general health of the subject, etc.

In certain embodiments an agent, e.g., 3-BrPA or an analog thereof may be used at the maximum tolerated dose or a sub-therapeutic dose or any dose there between, e.g., the lowest dose effective to achieve a therapeutic effect. Maximum tolerated dose (MTD) refers to the highest dose of a pharmacological or radiological treatment that can be administered without unacceptable toxicity, that is, the highest dose that has an acceptable risk/benefit ratio, according to sound medical judgment. In general, the ordinarily skilled practitioner can select a dose that has a reasonable risk/benefit ratio according to sound medical judgment. A MTD may, for example, be established in a population of subjects in a clinical trial. In certain embodiments an agent is administered in an amount that is lower than the MTD, e.g., the agent is administered in an amount that is about 10%, 20%, 30%, 40%, 50%, 60%, 70%, 80%, or 90% of the MTD.

It may be desirable to formulate pharmaceutical compositions, particularly those for oral or parenteral compositions, in unit dosage form for ease of administration and uniformity of dosage. Unit dosage form, as that term is used herein, refers to physically discrete units suited as unitary dosages for the subject to be treated; each unit containing a predetermined quantity of active agent(s) calculated to produce the desired therapeutic effect in association with an appropriate pharmaceutically acceptable carrier. In some embodiments a pharmaceutically acceptable unit dosage form contains a predetermined amount of an agent, e.g., 3-BrPA or an analog thereof, such amount being appropriate to treat a subject in need of treatment for a cancer.

It will be understood that a therapeutic regimen may include administration of multiple unit dosage forms over a period of time. In some embodiments, a subject is treated for between 1-7 days. In some embodiments a subject is treated for between 7-14 days. In some embodiments a subject is treated for between 14-28 days. In other embodiments, a longer course of therapy is administered, e.g., over between about 4 and about 10 weeks. In some embodiments multiple courses of therapy are administered. In some embodiments, treatment may be continued indefinitely. For example, a subject at risk of cancer recurrence may be treated for any period during which such risk exists. A subject may receive one or more doses a day, or may receive doses every other day or less frequently, within a treatment period. Treatment courses may be intermittent.

In some embodiments, an agent, e.g., 3-BrPA or an analog, thereof is provided in a pharmaceutical pack or kit comprising one or more containers (e.g., vials, ampoules, bottles) containing the 3-BrPA or analog thereof and, optionally, one or more other pharmaceutically acceptable ingredients. Optionally associated with such container(s) can be a notice in the form prescribed by a governmental agency regulating the manufacture, use or sale of pharmaceutical products, which notice reflects approval by the agency of manufacture, use or sale for human administration. The notice may describe, e.g., doses, routes and/or methods of administration, approved indications (e.g., cancers that the agent or pharmaceutical composition has been approved for use in treating), mechanism of action, or other information of use to a medical practitioner and/or patient. In some embodiments the notice specifies that the agent is to be used for treating tumors that have increased MCT1 expression, or equivalent language. In some embodiments a particular test for assessing MCT1 expression by a tumor is suggested or specified, e.g., as part of an indication. Different ingredients may be supplied in solid (e.g., lyophilized) or liquid form. Each ingredient will generally be suitable as aliquoted in its respective container or provided in a concentrated form. Kits may also include media for the reconstitution of lyophilized ingredients. The individual containers of the kit are preferably maintained in close confinement for commercial sale.

Similar considerations and embodiments regarding dose, administration route, pharmaceutical compositions, pharmaceutically acceptable salts, esters, etc., may be employed with regard to other pharmaceutically active agents of interest herein, e.g., MCT1 inhibitors, GAPDH inhibitors, glycolysis inhibitors, etc., and combinations thereof with 3-BrPA or an analog thereof.

One of ordinary skill in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure provides embodiments in which exactly one member of the group is present in, employed in, or otherwise relevant to a given product or process. The present disclosure also provides embodiments in which more than one, or all of the group members are present in, employed in, or otherwise relevant to a given product or process. Furthermore, it is to be understood that the present disclosure provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects described herein where appropriate. It is also contemplated that any of the embodiments or aspects or teachings can be freely combined with one or more other such embodiments or aspects whenever appropriate and regardless of where such embodiment(s), aspect(s), or teaching(s) appear in the present disclosure. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect can be explicitly excluded from the claims, regardless of whether the specific exclusion is recited in the specification. For example, any one or more agents, disorders, subjects, or combinations thereof, can be excluded.

Where the claims or description relate to a product (e.g., a composition of matter), it should be understood that methods of making or using the product according to any of the methods disclosed herein, and methods of using the product for any one or more of the purposes disclosed herein, are encompassed by the present disclosure, where applicable, unless otherwise indicated or unless it would be evident to one of ordinary skill in the art that a contradiction or inconsistency would arise. Where the claims or description relate to a method, it should be understood that product(s), e.g., compositions of matter, device(s), or system(s), useful for performing one or more steps of the method are encompassed by the present disclosure, where applicable, unless otherwise indicated or unless it would be evident to one of ordinary skill in the art that a contradiction or inconsistency would arise.

Where ranges are given herein, embodiments are provided in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, embodiments that relate analogously to any intervening value or range defined by any two values in the series are provided, and that the lowest value may be taken as a minimum and the greatest value may be taken as a maximum. Where a phrase such as “at least”, “up to”, “no more than”, or similar phrases, precedes a series of numbers herein, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated where applicable. A reasonable lower or upper limit may be selected or determined by one of ordinary skill in the art based, e.g., on factors such as convenience, cost, time, effort, availability (e.g., of samples, agents, or reagents), statistical considerations, etc. In some embodiments an upper or lower limit differs by a factor of 2, 3, 5, or 10, from a particular value, Numerical values, as used herein, include values expressed as percentages. For each embodiment in which a numerical value is prefaced by “about” or “approximately”, embodiments in which the exact value is recited are provided. For each embodiment in which a numerical value is not prefaced by “about” or “approximately”, embodiments in which the value is prefaced by “about” or “approximately” are provided. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. In some embodiments a method may be performed by an individual or entity. In some embodiments steps of a method may be performed by two or more individuals or entities such that a method is collectively performed. In some embodiments a method may be performed at least in part by requesting or authorizing another individual or entity to perform one, more than one, or all steps of a method. In some embodiments a method comprises requesting two or more entities or individuals to each perform at least one step of a method. In some embodiments performance of two or more steps is coordinated so that a method is collectively performed. Individuals or entities performing different step(s) may or may not interact. In some embodiments a request is fulfilled, e.g., a method or step is performed, in exchange for a fee or other consideration and/or pursuant to an agreement between a requestor and an individual or entity performing the method or step. It should also be understood that unless otherwise indicated or evident from the context, any product or composition described herein may be considered “isolated”. It should also be understood that, where applicable, unless otherwise indicated or evident from the context, any method or step of a method that may be amenable to being performed mentally or as a mental step or using a writing implement such as a pen or pencil, and a surface suitable for writing on, such as paper, may be expressly indicated as being performed at least in part, substantially, or entirely, by a machine, e.g., a computer, device (apparatus), or system, which may, in some embodiments, be specially adapted or designed to be capable of performing such method or step or a portion thereof.

Section headings used herein are not to be construed as limiting in any way. It is expressly contemplated that subject matter presented under any section heading may be applicable to any aspect or embodiment described herein.

Embodiments or aspects herein may be directed to any agent, composition, article, kit, and/or method described herein. It is contemplated that any one or more embodiments or aspects can be freely combined with any one or more other embodiments or aspects whenever appropriate. For example, any combination of two or more agents, compositions, articles, kits, and/or methods that are not mutually inconsistent, is provided. It will be understood that any description or exemplification of a term anywhere herein may be applied wherever such term appears herein (e.g., in any aspect or embodiment in which such term is relevant) unless indicated or clearly evident otherwise.

### EXAMPLES

### Methods and Materials

Materials.

Material were obtained from the following sources: antibody to SLC16A1 from Millipore, antibodies to RPS6, ACC, AMPK, phospho-ACC, phospho-AMPK and Raptor from Cell Signaling Technologies, HRP-conjugated anti-rabbit antibody from Santa Cruz Biotechnology, lactate dehydrogenase from Roche, lactic acid and 3-BrPA from Acros Organics, RPMI-1640 media, glycine-hydrazine solution, pyruvate, l-lactate, d-lactate puromycin, and polybrene from Sigma, blasticidin from Invivogen, ¹⁴C-3BrPA from Moravek Biosciences, Matrigel and Cell-Tak from BD Biosciences, IMDM from US Biological. MCT1 cDNA was cloned into pMXs-IRES-blasticidin vector using the following primers: AGGGATCCATGCCACCAGCAGTTGGAGG/AGGCGGCCGCTCAGACTGGACTTTCCT CCTCCTTG (SEQ ID NO: 2). MCT1 cDNA was cloned into PLJMI-puro vector using the following primers: ATTGAATTCTATGCCACCAGCAGTTGGAGG/ATTAATTCGTTCGAATCAGACTGGA CTTTCCTCCTCCTTG (SEQ ID NO: 3). Lentiviral shRNAs were obtained from the The RNAi Consortium (TRC) collection of the Broad Institute. The TRC numbers for the shRNAs used are TRCN0000072186 (GFP), TRCN0000038339 (MCT1_{—}1) and TRCN0000038340 (MCT1_{—}2).

Cell Culture and Virus Transduction.

KBM7 cells were cultured in IMDM supplemented with 10% IFS and penicillin/streptomycin. All other cell lines in this study were cultured in RPMI supplemented with 10% FBS. KBM7 cell line and MDA-MB-231/Sk-Br-3 cell lines stably overexpressing human MCT1 or GFP were generated by infection with lentiviruses expressing the corresponding cDNA and selected for blasticidin (10 μg/ml) or puromycin resistance (4 ng/ml) for 3 days, respectively. Similarly BT-20 and BT-549 expressing shRNAs for MCT1 were generated by infection with lentiviruses expressing the corresponding shRNAs and selected for puromycin resistance. Cells were cultured in 6-well plates and spin-infected via a 30-min spin at 2,250 rpm in media containing 4 μg/ml polybrene.

Haploid Cell Screening.

A haploid cell genetic screen with 3-BrPA was performed using 100 million mutagenized KBM7 cells as described previously²⁹. Mutagenized haploid KBM-7 cells were exposed to 50 μM 3-BrPA for 3 weeks. Surviving clones were harvested and genomic DNA was isolated and insertions were amplified. The sequences flanking retroviral insertion sites were mapped to the human genome using inverse PCR followed by Ilium in a sequencing. Genomic regions with a high density of insertions were identified using the proximity index for a given insertion. Additionally, the statistical enrichment of insertions at a given locus in the selected population was calculated by comparing the number of inactivating insertions to those in the untreated control dataset via a Fisher's Exact Test. Individual clones were isolated among 50 different clones and genomic DNA for individually selected clones were isolated using a genomic DNA isolation kit (Qiagen). Genomic insertions were identified by inverse PCR and subsequent sequencing as described previously²⁴.

Metabolic Assays.

The bioenergetic profiles of KBM7 and MDA-MB-231 cells in response to 3-BrPA were determined using a Seahorse Bioscience XF24 Extracellular Flux Analyzer (Seahorse Bioscience). For the indicated experiments, 250,000 KBM7 or 40,000 MDA-MB-231 cells seeded in Seahorse tissue culture plates using unbuffered RPMI (10 mM Glucose). KBM7 cells were attached to the plates using CellTak (Clontech) one hour prior to the start of the experiment. For AUC (area under the curve) OCR and ECAR measurements, three consecutive readings were performed for each cell line. For experiments where 3-BrPA is used, after 3 consecutive readings, 50 μM 3-BrPA was injected through port A and ECAR and OCAR levels were measured. Lactate production was measured as described previously⁵⁰. For ATP assays, 20,000 cells were seeded and treated for 30 min with indicated amounts of 3-BrPA and ATP levels were determined using a luciferase-based assay (Promega). For metabolite measurements, 10 million wild type and MCT1 null KBM7 cells were cultured for 1 hour in presence of 50 μM 3-BrPA before metabolite extraction. Then, cells were rapidly washed three times with cold PBS, and then metabolites were extracted by addition of 80% ice-cold methanol followed after incubation on dry ice for 15 min. Endogenous metabolite profiles were obtained using LC-MS as described⁵¹. Metabolite levels (n=3) were normalized to cell number.

3-BrPA Uptake Assay.

Wild type KBM7 or MCT1-null cells were seeded in HBSS and exposed for up to 20 minutes to 100 μM ¹⁴C-labeled 3-BrPA (6.8 mCi/mmole) (Moravek) with or without competitor molecules like 3-BrPA, Pyruvate, L-Lactate and D-Lactate. Cells were washed with cold HBSS, lysed in a NaOH buffer, and uptake measured using a liquid scintillation counter.

Cell Survival Assays.

Cell lines (5,000-20,000) were seeded in 96-well plates and treated with indicated amounts of 3-BrPA. After 3 days of treatment, CellTiter-Glo (Promega) and/or CyQuant (Invitrogen) were used to measure survival percentage for each concentration compared to untreated cells. FACS analysis with Annexin V and 7AAD staining was performed according to manufacturer's manual (BD Pharmingen).

Correlation Analysis.

15 cell lines (BT-474, BT-549, CAK1-1, CAL-51, HCC-70, HS578T, JURKAT, MDA-MB-157, MDA-MB-231, MDA-MB-453, MDA-MB-468, PC-3, SK-BR-3, T47D, ZR-75-1) were treated with 3-BrPA (0-300 μM). After 3 days, cell viability was quantified via CellTiter-Glo assay and an IC₅₀ from the resulting dose-response curve was interpolated using a nonlinear regression model. Transcriptome-wide normalized mRNA levels from gene expression profiling experiments performed on the Affymetrix Human Genome U133 Plus 2.0 chip were obtained from the CCLE for all 15 cell lines. The mRNA expression pattern across all 15 samples for each of the genes was then correlated with the IC₅₀ values. Similar correlation analysis was performed using OCR/ECAR values for each cell line.

Immunoblotting and Immunohistochemistry.

Briefly, cells were rinsed twice in ice-cold PBS and harvested in a standard lysis buffer containing 50 mM Hepes, pH 7.4, 40 mM NaCl, 2 mM EDTA, 1.5 mM orthovanadate, 50 mM NaF, 10 mM pyrophosphate, 10 mM glycerophosphate, protease inhibitors (Roche) and 1% Triton-X-100. Proteins from total lysates were resolved by 8-12% SDS-PAGE, and analyzed by immunoblotting as described using anti-MCT1 (Millipore; Ab3540), anti-RPS6 and anti-raptor antibodies (1:1000)⁵⁰. For quantitation, ImageJ software was used and the signals were normalized using an equal loading control (RPS6/Raptor). Immunohistochemistry was performed on formalin-fixed paraffin embedded sections using a boiling Dako antigen retrieval method (Dako). A 1:500 dilution of the anti-MCT1 antibody was used for staining.

Mouse Studies.

All animal studies and procedures were approved by the MIT Institutional Animal Care and Use Committee. 6-10 week old nude mice (Taconic) were used for generating all xenografts. In subcutaneous xenografts, mice were injected at two sites in the dorsal region, under isofluorane anesthesia with 100 μl/injection of tumor cell suspension in RPMI with 20% matrigel. 2.5 million MDA-MB-231 cells were injected. After 2 weeks, tumors were measured and mice were separated for PBS and 3-BrPA treatment (8 mg/kg). After 3 weeks, tumors were harvested, their dimensions were measured with a caliper and tumor volume estimated according to the formula: (0.5*W*L*L). Tumors were fixed in formalin for later processing.

### Example 1

### Genetic Screen in Near-Haploid Cells Identifies MCT1 as the Main Determinant of 3-Bromopyruvate Sensitivity

We undertook a loss of function genetic screen for genes that affect the sensitivity of cancer cells to 3-bromopyruvate (3-BrPA), a drug candidate under clinical development^{13,14}. 3-BrPa has cytotoxic effects and decreases cellular energy levels by inhibiting glycolysis in a poorly understood fashion¹⁵. 3-BrPA can inhibit several glycolytic¹⁶⁻¹⁸ and non-glycolytic enzymes¹⁹⁻²³ and, given its simple structure, is likely to have more than one direct protein target within cells. Here, we identify the MCT1 transporter as the main determinant of 3-BrPa uptake and sensitivity, leading us to propose the therapeutic strategy of using MCT1-mediated transport to deliver toxic molecules to glycolytic tumors.

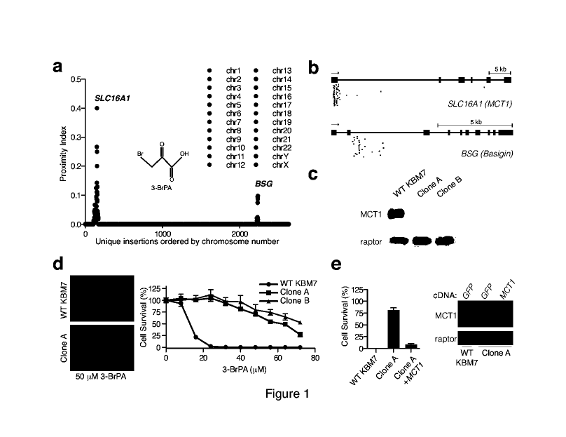

Insertional mutagenesis in haploid or near haploid mammalian cells has enabled genome-wide loss of function screens for genes underlying basic cellular physiology²⁴⁻²⁶. For example, screens in the near-haploid KBM7 human cell line identified the host factors necessary for the cytotoxic effects of several viruses and microbial toxins²⁷⁻²⁹. To apply this approach to the study of 3-BrPA, we used retroviral infection to create a library of mutagenized haploid KBM7 cells containing ˜70 million insertions, which covered approximately 98% of all genes expressed in KBM7 cells²⁹. The mutagenized cells were treated with 3-BrPA and the surviving cells were expanded as a pool. Using massively parallel sequencing, insertions in the resistant population were mapped to the human genome. A proximity index analysis was used to identify genomic regions that contained multiple gene-trap insertions in close proximity. SLC16A1 (MCT1) and BSG (Basigin) were the two most frequently inactivated genes (FIG. 1A) and had the highest degree of insertional enrichment compared to the unselected control cells (p=4.7E-121 and p=5E-29, respectively) (Supplemental FIG. 1A). The highest scoring gene, SLC16A1, encodes MCT1, an H+ linked monocarboxylate transporter that excretes lactate from cells and is highly upregulated in a subset of cancers³⁰⁻³⁵. The second highest scoring gene, Basigin, is a chaperone necessary for escorting MCT1 to the plasma membrane^{36,37}. To enable the in depth study of the effects of MCT1 loss, we isolated two clones (Clone A and B) that carry insertions in the first intron of the MCT1 gene (FIG. 1B) and in which MCT1 protein is undetectable by immunoblotting (FIG. 1C). Consistent with the screening results, the MCT1-null cells were completely resistant to doses of 3-BrPA (FIG. 1D) that in parental KBM7 cells induce cell death accompanied by caspase-3 activation (Supplemental FIG. 1B). Importantly, re-expression of MCT1 in the MCT1-null cells nearly completely restored their sensitivity to 3-BrPA (FIG. 1E). Thus, these data strongly point to MCT1 as an important determinant of 3-BrPA sensitivity in KBM7 cells.

### Example 2

### MCT1 is Necessary for the Cellular Uptake of 3-BrPA and Directly Transports 3-BrPA

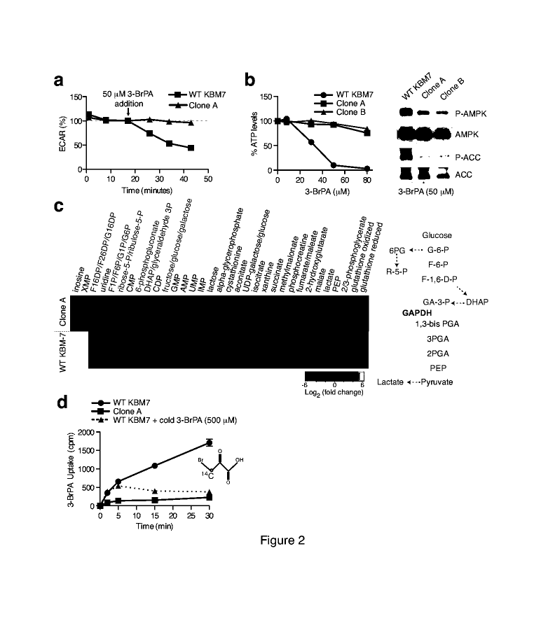

To begin to understand how loss of MCT1 confers 3-BrPA resistance, we examined the effects of 3-BrPA on the metabolism of parental and MCT1-null KBM7 cells. In the absence of 3-BrPA, there were no differences in lactate production or oxygen consumption between the cell types (Supplemental FIG. 2), suggesting that MCT1 loss does not alter basal energy metabolism to any great extent. In contrast, 3-BrPA caused a substantial decrease in the extracellular acidification rate (ECAR), a proxy for lactate production, and total ATP levels (FIGS. 2A and 2B) of parental, but not MCT1-null, KBM7 cells. Consistent with these findings, 3-BrPA did not affect AMPK and ACC phosphorylation, markers of energy stress³⁸, in MCT1-null cells while robustly increasing them in the wild type counterparts (FIG. 2B). To more completely characterize the metabolic state of cells in response to 3-BrPA, we metabolically profiled wild type and MCT1-null KBM7 cells treated with 3-BrPA. Relative to MCT1-null cells, in wild type KBM7 cells 3-BrPA caused an accumulation of the glycolytic intermediates that precede glyceraldehyde 3-phosphate, a substrate for glyceraldehyde phosphate dehydrogenase (GAPDH), but a depletion of those that come after. Furthermore, 3-BrPA treated wild type KBM7 cells accumulated intermediates of the pentose phosphate pathway, which branches off above the GAPDH step of glycolysis (FIG. 2C). Thus, even though several glycolytic enzymes can be inhibited by 3-BrPA in vitro, including hexokinase^{39,40}, lactate dehydrogenase¹⁸, glyceraldehyde phosphate dehydrogenase¹⁶, succinate dehydrogenase^{17,41}, aldolase⁴², and pyruvate kinase^{43,44}, our metabolite profiling strongly implicates GAPDH inhibition as the cause of its anti-glycolytic effects (FIG. 2C). Altogether, these data show that MCT1-null KBM7 cells are remarkably resistant to the metabolic effects of 3-BrPA, suggesting that 3-BrPA might not enter cells in the absence of MCT1 and implicating MCT1 as a 3-BrPA transporter.

Indeed, compared to parental KBM7 cells, MCT1-null cells did not take up ¹⁴C-labeled 3-BrPA (FIG. 2D). Unlabeled 3-BrPA and, to a lesser extent, known MCT1 substrates such as lactate and pyruvate, effectively competed with the uptake of radiolabeled 3-BrPA, demonstrating that the transport is specific (Supplemental FIG. 3). Moreover, consistent with the pH dependence of MCT1 transport^{30,45}, a reduction in extracellular pH enhanced 3-BrPA uptake (Supplemental FIG. 3). Thus, MCT1 is necessary for the cellular uptake of 3-BrPA and, given its capacity to transport monocarboxylates⁴⁵, likely directly transports 3-BrPA.

### Example 3

### MCT1 Expression Levels in Cancer Cells Predicts their Sensitivity to 3-BrPA

Considering that MCT1 loss confers resistance to 3-BrPA in KBM7 cells, we asked if MCT1 expression levels in cancer cells might predict their sensitivity to 3-BrPA. In a panel of 15 cancer cell lines, we determined IC₅₀ values for 3-BrPA-induced cell death and correlated them with transcriptome-wide mRNA expression data from the Cancer Cell Line Encyclopedia (CCLE)⁴⁶. Of the 20,000 mRNAs examined, MCT1 mRNA levels were the single best predictor of 3-BrPA sensitivity (r=−0.89, p=1.4E-5) (FIG. 3A). We next asked whether MCT1 expression can also predict 3-BrPA sensitivity within a single cancer type and focused on breast cancer lines because they exhibit a particularly wide range of MCT1 expression levels (Supplemental FIG. 4). Indeed, breast cancer lines with high MCT1 protein levels are sensitive to 3-BrPA, whereas those with low to no MCT1 are resistant to even high concentrations of 3-BrPA (FIG. 3B). Stable expression of MCT1 in two breast cancer lines with low MCT1 expression (MDA-MB-231 and SK-BR-3) was sufficient to sensitize them to 3-BrPA (FIG. 3C). Additionally, as in KBM7 cells, MCT1 expression did not alter lactate production or oxygen consumption, but it did enhance ¹⁴C-3-BrPA uptake (FIG. 3D). Lastly, the partial suppression by RNAi of MCT1 expression was sufficient to confer resistance to 3-BrPA to cell lines with high levels of MCT1 (BT-20, BT-549) (FIG. 3E).

To test if MCT1 expression can affect the sensitivity of established tumors to 3-BrPA, parental MDA-MB-231 cells, which express low levels of MCT1 (Supplemental FIG. 4), were injected subcutaneously into the left flanks of Nude mice, while MDA-MB-231 cells stably expressing MCT1 were injected into the contralateral flanks of the same animals. We allowed palpable subcutaneous tumors to form for 2 weeks before beginning 3-BrPA administration. After 3 weeks of 3-BrPA treatment, tumors with forced MCT1 expression were significantly smaller than those that were untreated or treated with 3-BrPA but expressing a control protein (GFP) (FIG. 3F). These results indicate that MCT1 expression is sufficient to sensitize pre-formed tumors to 3-BrPA treatment and has predictive value for determining 3-BrPA sensitivity in vivo.

### Example 4

### MCT1 Expression in Cancer Cells Correlates with Elevated Glycolysis

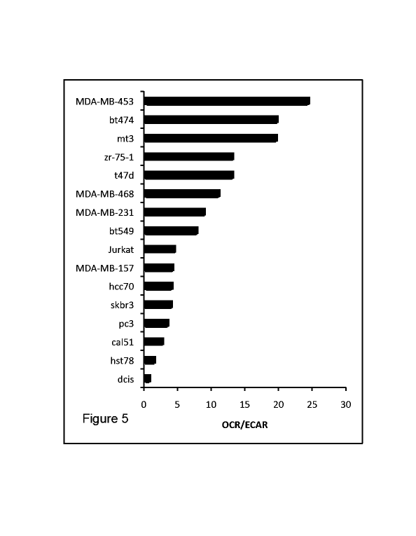

We additionally examined if cancer cells with high levels of MCT1 expression share any metabolic properties. Using the oxygen consumption rate (OCR) to ECAR ratio as a measure of the relative contributions of OXPHOS and glycolysis to cellular energy production, we compared OCR/ECAR ratios from 15 cancer cell lines with genome-wide expression data obtained from the CCLE (FIG. 5). Interestingly, along with two glycolytic enzymes (LDHB and PGM1), MCT1 was amongst the genes whose expression most strongly and significantly correlated with lower OCR/ECAR ratios (FIG. 4, Supplemental FIG. 4). This finding indicates that tumors which exhibit the highest rates of glycolysis are more likely to have elevated levels of MCT1 and therefore will be more sensitive to 3-BrPA treatment (FIG. 4).

Our results predict that MCT1 expression levels will serve as a biomarker for identifying tumors likely to respond to 3-BrPA treatment. Furthermore, as we find that MCT1 expression correlates with elevated glycolysis, we propose to enhance the efficacy of 3-BrPA by concomitant treatment with glycolytic inhibitors so as to exploit the high glycolytic demand of tumors and the cancer-enriched expression of MCT1. Small molecule inhibitors of MCT1 that inhibit lactate export from cancer cells are in development and show promise as anti-anti-cancer therapies^{10,47}. While this approach requires that MCT1 be expressed and important for cancer cell survival^{10,47}, 3-BrPA treatment is distinct in that it requires only MCT1 expression to be efficacious. For example, KBM7 cells are sensitive to 3-BrPA, but complete loss of MCT1 does not affect their viability. Like MCT1, a number of other transporters are also upregulated in subsets of cancers^{48,49}. Developing toxic molecules that, in a fashion analogous to 3-BrPA, exploit these transporters to selectively enter and target cancer cells, is a promising approach to treatment of these cancers.

### Example 5

### Synergistic Effect of Combined 3-BrPA and GAPDH Inhibition

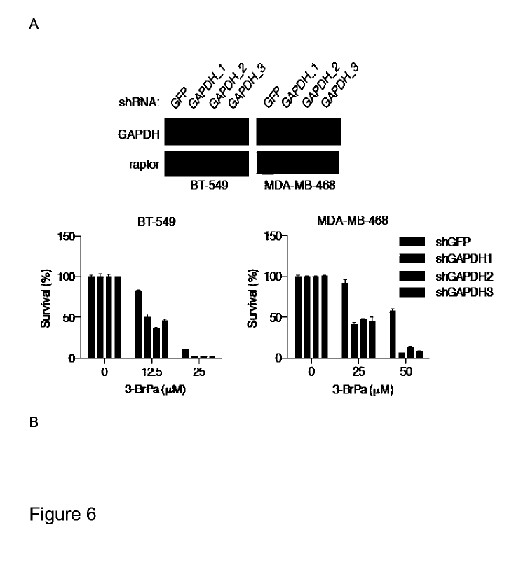

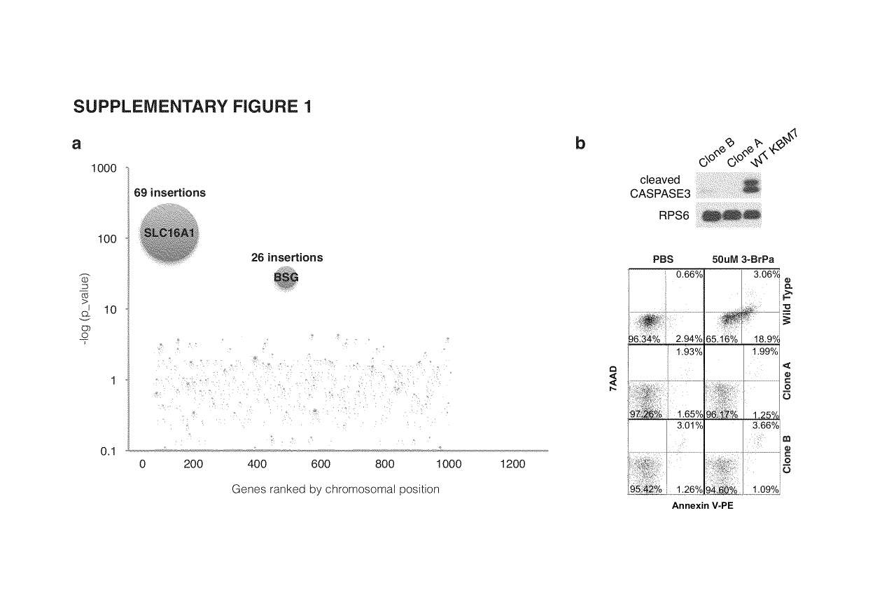

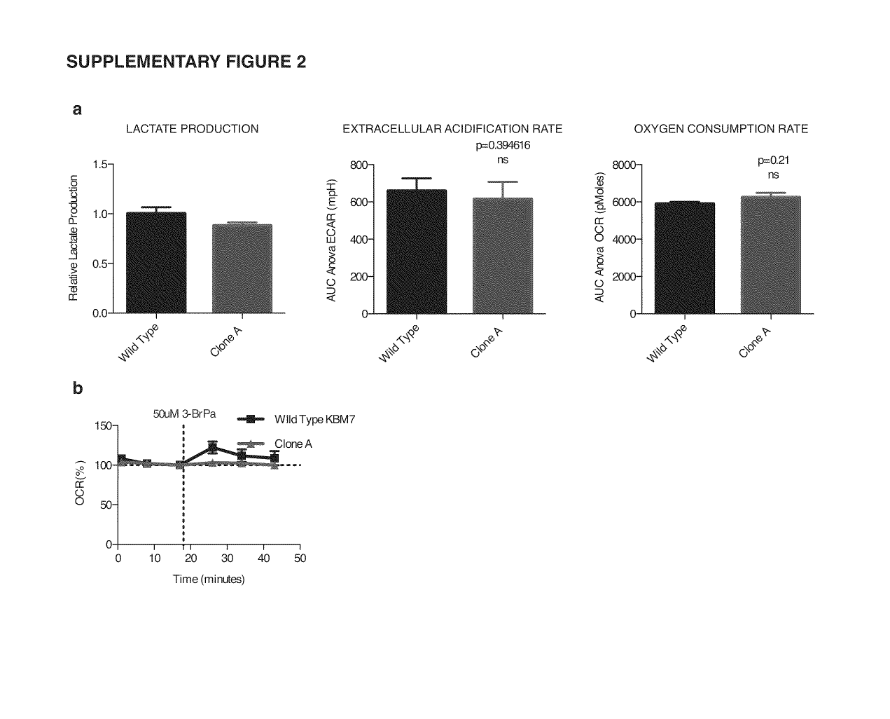

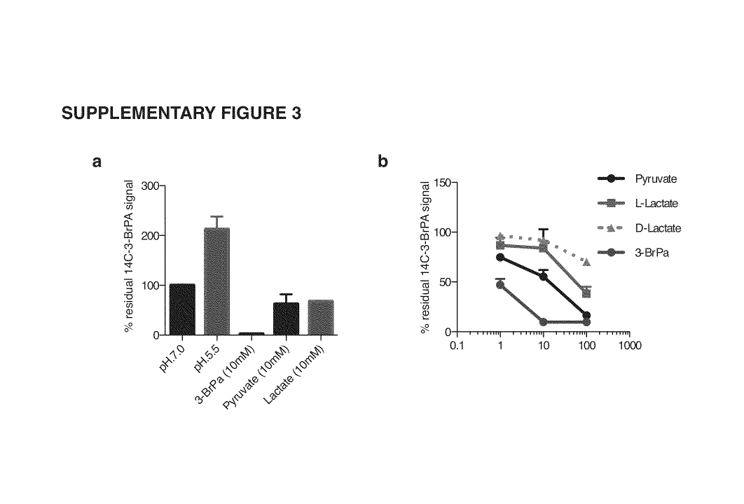

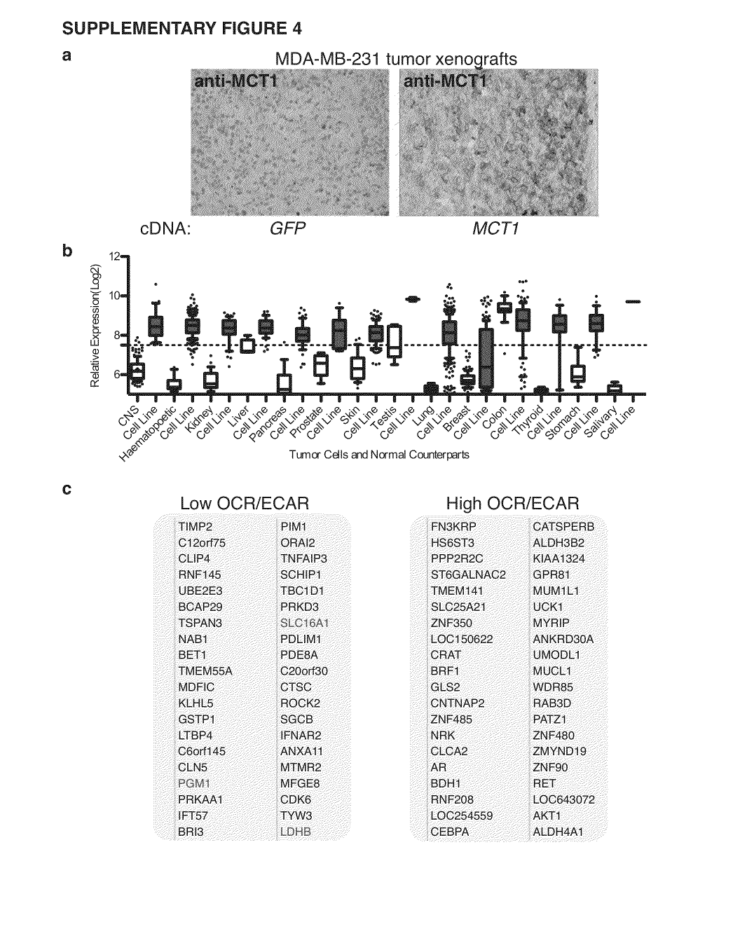

As described in Example 2, metabolic profiling indicated that GAPDH is the likely primary target responsible for the effect of 3-BrPA on tumor cells. This result suggested that inhibiting GAPDH might have synthetic lethal effects in combination with 3-BrPA. Indeed, as shown in FIG. 6(B), suppression of GAPDH expression by RNAi (using three different short hairpin RNAs targeted to GAPDH), markedly increased the sensitivity of tumor cells to the inhibitory effects of 3-BrPA on tumor cell survival/proliferation in the two different tumor cell lines tested. Thus, combined treatment with 3-BrPA and GAPDH inhibition has synthetic lethal effects.

### REFERENCES

1. Vander Heiden, M. G. Targeting cancer metabolism: a therapeutic window opens. Nat Rev Drug Discov 10, 671-684, doi:nrd3504 [pii] 10.1038/nrd3504 (2011).
2 Thompson, C. B. Rethinking the Regulation of Cellular Metabolism. Cold Spring Harb Symp Quant Biol, doi:sqb.2012.76.010496 [pii] 10.1101/sqb.2012.76.010496 (2012).
3 DeBerardinis, R. J. & Thompson, C. B. Cellular metabolism and disease: what do metabolic outliers teach us? Cell 148, 1132-1144, doi:S0092-8674(12)00232-2 [pii] 10.1016/j.cell.2012.02.032 (2012).
4 Tennant, D. A., Duran, R. V. & Gottlieb, E. Targeting metabolic transformation for cancer therapy. Nat Rev Cancer 10, 267-277, doi:nrc2817 [pii] 10.1038/nrc2817 (2010).
5 Warburg, O. On respiratory impairment in cancer cells. Science 124, 269-270 (1956).
6 Pelicano, H., Martin, D. S., Xu, R. H. & Huang, P. Glycolysis inhibition for anticancer treatment. Oncogene 25, 4633-4646, doi:1209597 [pii] 10.1038/sj.onc.1209597 (2006).
7 Xu, R. H. et al. Inhibition of glycolysis in cancer cells: a novel strategy to overcome drug resistance associated with mitochondrial respiratory defect and hypoxia. Cancer Res 65, 613-621, doi:65/2/613 [pii] (2005).
8 Sonveaux, P. et al, Targeting lactate-fueled respiration selectively kills hypoxic tumor cells in mice. J Clin Invest 118, 3930-3942, doi:36843 [pii] 10.1172/JC136843 (2008).
9 Vander Heiden, M. G. et al. Identification of small molecule inhibitors of pyruvate kinase M2. Biochem Pharmacol 79, 1118-1124, doi:S0006-2952(09)01060-0 [pii] 10.1016/j.bcp.2009.12.003 (2010).
10 Le Floch, R. et al. CD147 subunit of lactate/H+ symporters MCT1 and hypoxia-inducible MCT4 is critical for energetics and growth of glycolytic tumors. Proc Natl Acad Sci USA 108, 16663-16668, doi:1106123108 [pii] 10.1073/pnas.1106123108 (2011).
11 Wood, T. E. et al. A novel inhibitor of glucose uptake sensitizes cells to FAS-induced cell death. Mol Cancer Ther 7, 3546-3555, doi:7/11/3546 [pii] 10.1158/1535-7163.MCT-08-0569 (2008).
12 Stein, M. et al. Targeting tumor metabolism with 2-deoxyglucose in patients with castrate-resistant prostate cancer and advanced malignancies. Prostate 70, 1388-1394, doi:10.1002/pros.21172 (2010).
13 Pedersen, P. L. 3-bromopyruvate (3BP) a fast acting, promising, powerful, specific, and effective “small molecule” anti-cancer agent taken from labside to bedside: introduction to a special issue. J Bioenerg Biomembr 44, 1-6, doi:10.1007/s10863-012-9425-4 (2012).
14 Ko, Y. H. et al. A translational study “case report” on the small molecule “energy blocker” 3-bromopyruvate (3BP) as a potent anticancer agent: from bench side to bedside. J Bioenerg Biomembr 44, 163-170, doi:10.1007/s10863-012-9417-4 (2012).
15 Shoshan, M. C. 3-bromopyruvate: Targets and outcomes. J Bioenerg Biomembr, doi:10.1007/s10863-012-9419-2 (2012).
16 Ganaphthy-Kanniappan, S. et al. Glyceraldehyde-3-phosphate dehydrogenase (GAPDH) is pyruvylated during 3-bromopyruvate mediated cancer cell death. Anticancer Res 29, 4909-4918, doi:29/12/4909 [pii] (2009).
17 Pereira da Silva, A. P. et al. Inhibition of energy-producing pathways of HepG2 cells by 3-bromopyruvate, Biochem J 417, 717-726, doi:BJ20080805 [pii] 10.1042/BJ20080805 (2009).
18 Dell'Antone, P. Targets of 3-bromopyruvate, a new, energy depleting, anticancer agent. Med Chem 5, 491-496, doi:MC-Abs-02 [pii] (2009).
19 Dell'Antone, P. Inactivation of H+-vacuolar ATPase by the energy blocker 3-bromopyruvate, a new antitumour agent. Life Sci 79, 2049-2055, doi:S0024-3205(06)00519-4 [pii] 10.1016/j.lfs.2006.06.043 (2006).
20 Blessinger, K. J. & Tunnicliff, G. Kinetics of inactivation of 4-aminobutyrate aminotransferase by 3-bromopyruvate. Biochem Cell Biol 70, 716-719 (1992).
21 Tunnicliff, G. & Ngo, T. T. Mechanism of inactivation of brain glutamic decarboxylase by 3-bromopyruvate. Int J Biochem 9, 249-252 (1978).
22 Arendt, T., Schugens, M. M. & Marchbanks, R. M. Reversible inhibition of acetylcholine synthesis and behavioural effects caused by 3-bromopyruvate. Neurochem 55, 1474-1479 (1990).
23 Jardim-Messeder, D., Camacho-Pereira, J. & Galina, A. 3-Bromopyruvate inhibits calcium uptake by sarcoplasmic reticulum vesicles but not SERCA ATP hydrolysis activity. Int J Biochem Cell Biol 44, 801-807, doi:S1357-2725(12)00046-5 [pii] 10.1016/j.biocel.2012.02.002 (2012).
24 Carette, J. E. et al. Haploid genetic screens in human cells identify host factors used by pathogens. Science 326, 1231-1235, doi:326/5957/1231 [pii]10.1126/science.1178955 (2009).
25 Layton, J. E. Undertaking a successful gynogenetic haploid screen in zebrafish. Methods Mol Biol 546, 31-44, doi: 10.1007/978-1-60327-977-2_{—}3 (2009).
26 Elling, U. et al. Forward and reverse genetics through derivation of haploid mouse embryonic stem cells. Cell Stem Cell 9, 563-574, doi:S1934-5909(11)00492-9 [pii] 10.1016/j.stem.2011.10.012 (2011).
27 Carette, J. E. et al. Ebola virus entry requires the cholesterol transporter Niemann-Pick C1. Nature 477, 340-343, doi:nature10348 [pii] 10.1038/nature10348 (2011).
28 Guimaraes, C. P, et al. Identification of host cell factors required for intoxication through use of modified cholera toxin. J Cell Biol 195, 751-764, doi:jcb.201108103 [pii] 10.1083/jcb.201108103 (2011).
29 Carette, J. E. et al. Global gene disruption in human cells to assign genes to phenotypes by deep sequencing. Nat Biotechnol 29, 542-546, doi:nbt.1857 [pii] 10.1038/nbt.1857 (2011).
30 Morris, M. E. & Felmlee, M. A. Overview of the proton-coupled MCT (SLC16A) family of transporters: characterization, function and role in the transport of the drug of abuse gamma-hydroxybutyric acid. AAPS J 10, 311-321, doi:10.1208/s12248-008-9035-6 (2008).
31 Pinheiro, C. et al. Monocarboxylate transporter 1 is up-regulated in basal-like breast carcinoma. Histopathology 56, 860-867, doi:HIS3560 [pii] 10.1111/j.1365-2559.2010.03560.x (2010).
32 Pinheiro, C. et al. Monocarboxylate transporters 1 and 4 are associated with CD147 in cervical carcinoma. Dis Markers 26, 97-103, doi:TX61467X4308U036 [pii] 10.3233/DMA-2009-0596 (2009).
33 Mathupala, S. P., Parajuli, P. & Sloan, A. E. Silencing of monocarboxylate transporters via small interfering ribonucleic acid inhibits glycolysis and induces cell death in malignant glioma: an in vitro study. Neurosurgery 55, 1410-1419; discussion 1419 (2004).
34 Koukourakis, M. I., Giatromanolaki, A., Bougioukas, G. & Spyridis, E. Lung cancer: a comparative study of metabolism related protein expression in cancer cells and tumor associated stroma. Cancer Biol Ther 6, 1476-1479, doi:1635 [pii] (2007).
35 Pinheiro, C. et al. Increased expression of monocarboxylate transporters 1, 2, and 4 in colorectal carcinomas. Virchows Arch 452, 139-146, doi:10.1007/s00428-007-0558-5 (2008).
36 Poole, R. C. & Halestrap, A. P. Interaction of the erythrocyte lactate transporter (monocarboxylate transporter 1) with an integral 70-kDa membrane glycoprotein of the immunoglobulin superfamily. J Biol Chem 272, 14624-14628 (1997).
37 Kirk, P. et al. CD147 is tightly associated with lactate transporters MCT1 and MCT4 and facilitates their cell surface expression. EMBO J 19, 3896-3904, doi:10.1093/emboj/19.15.3896 (2000).
38 Mihaylova, M. M. & Shaw, R. J. The AMPK signalling pathway coordinates cell growth, autophagy and metabolism. Nat Cell Biol 13, 1016-1023, doi:ncb2329 [pii] 10.1038/ncb2329 (2011).
39 Rodrigues-Ferreira, C., da Silva, A. P. & Galina, A. Effect of the antitumoral alkylating agent 3-bromopyruvate on mitochondrial respiration: role of mitochondrially bound hexokinase. J Bioenerg Biomembr, doi:10.1007/s10863-012-9413-8 (2012).
40 Ko, Y. H. et al. Advanced cancers: eradication in all cases using 3-bromopyruvate therapy to deplete ATP. Biochem Biophys Res Commun 324, 269-275, doi:S0006-291X(04)02062-5 [pii] 10.1016/j.bbrc.2004.09.047 (2004).
41 Sanborn, B. M., Felberg, N. T. & Hollocher, T. C. The inactivation of succinate dehydrogenase by bromopyruvate. Biochim Biophys Acta 227, 219-231 (1971).
42 Meloche, H. P., Luczak, M. A. & Wurster, J. M. The substrate analog, bromopyruvate, as both a substrate and alkylating agent for 2-keto-3-deoxy-6-phosphogluconic aldolase. Kinetic and stereochemical studies. J Biol Chem 247, 4186-4191 (1972).
43 Yun, S. L. & Suelter, C, H. Modification of yeast pyruvate kinase by an active site-directed reagent, bromopyruvate. J Biol Chem 254, 1811-1815 (1979).
44 Acan, N. L. & Ozer, N. Modification of human erythrocyte pyruvate kinase by an active site-directed reagent: bromopyruvate. J Enzyme Inhib 16, 457-464 (2001).
45 Halestrap, A. P. The monocarboxylate transporter family—Structure and functional characterization. IUBMB Life 64, 1-9, doi:10.1002/iub.573 (2012).
46 Barretina, J. et al. The Cancer Cell Line Encyclopedia enables predictive modelling of anticancer drug sensitivity. Nature 483, 603-607, doi:nature11003 [pii] 10.1038/nature11003 (2012).
47 Murray, C. M. et al. Monocarboxylate transporter MCT1 is a target for immunosuppression. Nat Chem Biol 1, 371-376 (2005).
48 Ganaphthy, V., Thangaraju, M. & Prasad, P. D. Nutrient transporters in cancer: relevance to Warburg hypothesis and beyond. Pharmacol Ther 121, 29-40, doi:S0163-7258(08)00186-1 [pii] 10.1016/j.pharmthera.2008.09.005 (2009).
49 Gupta, N. et al. Upregulation of the amino acid transporter ATB0+(SLC6A14) in colorectal cancer and metastasis in humans. Biochim Biophys Acta 1741, 215-223, doi:S0925-4439(05)00049-9 [pii] 10.1016/j.bbadis.2005.04.002 (2005).
50 Possemato, R. et al. Functional genomics reveal that the serine synthesis pathway is essential in breast cancer. Nature 476, 346-350, doi:nature10350 [pii] 10.1038/nature10350 (2011).
51 Finley, L. W. et al. Skeletal muscle transcriptional coactivator PGC-1 alpha mediates mitochondrial, but not metabolic, changes during calorie restriction. Proc Natl Acad Sci USA 109, 2931-2936, doi:1115813109 [pii] 10.1073/pnas.1115813109 (2012).

1.-6. (canceled)

7. A method of treating a subject in need of treatment for a tumor, the method comprising: (a) determining that the subject's tumor has increased expression of the MCT1 gene; and (b) treating the subject with 3-bromopyruvate (3-BrPA) or an analog thereof.

8. The method of claim 7, wherein determining that tumor has increased expression of the MCT1 gene comprises (a) determining the level of an MCT1 gene product in the tumor or a sample obtained therefrom; and (b) comparing the level with a reference level of the MCT1 gene product.

9. The method of claim 8, wherein the reference level is a level of the gene product in non-tumor tissue or non-tumor cells.

10. The method of claim 8, wherein the reference level is a level of the gene product in tumor tissue or tumor cells that are sensitive to 3-BrPA.

11. The method of claim 8, wherein the MCT1 gene product is a MCT1 mRNA or a MCT1 polypeptide.

12. The method of claim 8, wherein the MCT1 gene product is a MCT1 polypeptide, and wherein the level is determined by a method comprising: contacting the tumor, tumor cell, or sample with a detection reagent; and detecting the MCT1 gene product based on detecting the detection reagent.

13. (canceled)

14. The method of claim 8, wherein the MCT1 gene product is a MCT1 polypeptide, and the level is determined by performing immunohistochemistry (IHC).

15-20. (canceled)

21. The method of claim 8, wherein the compound is 3-BrPA.

22. (canceled)

23. The method of claim 8, wherein the tumor is a carcinoma.

24. The method of claim 8, wherein the tumor is a liver tumor, breast tumor, glioblastoma, colon, or cervical tumor.

25-49. (canceled)

50. A method of identifying a candidate agent for modulating sensitivity of a cell to 3-BrPA or an analog thereof, the method comprising: (a) providing a test agent; and (b) determining whether the test agent modulates expression or activity of a MCT1 gene product, wherein the test agent is identified as a candidate agent for modulating sensitivity of a cell to 3-BrPA or an analog thereof if the test agent modulates expression or activity of a MCT1 gene product.

51. The method of claim 50, wherein determining whether the test agent modulates expression or activity of an MCT1 gene product comprises (i) contacting the test agent with one or more cells that express a MCT1 gene product; and (ii) measuring the level of expression or activity of the MCT1 gene product; wherein an alteration in expression or activity of the MCT1 gene product relative to control cell(s) not exposed to the test agent is indicative that the test agent modulates expression or activity of the MCT1 gene product.

52-69. (canceled)

70. A method of testing the ability of an agent to inhibit the survival and/or proliferation of a cell comprising (a) contacting one or more test cells with an agent, wherein the one or more test cells has increased expression of a transporter as compared to one or more control cells; (b) assessing the level of inhibition of the survival and/or proliferation of the one or more test cells by the agent; and (c) comparing the level of inhibition of the survival and/or proliferation of the one or more test cells by the agent with the level of inhibition of survival and/or proliferation of control cells by the agent.

71. The method of claim 70, wherein the transporter is characterized in that it is expressed at increased levels by at least some tumors as compared with non-tumor cells of the same cell type or tissue of origin.

72. The method of claim 70, further comprising (d) identifying the agent as a candidate anti-tumor agent if the level of inhibition of the survival and/or proliferation of the one or more test cells by the agent is greater than the level of inhibition of survival and/or proliferation of control cells by the agent

73. (canceled)

74. The method of claim 72, wherein the one or more test cells express the transporter or mRNA encoding the transporter at a level at least five times as great as the one or more control cells.

75-82. (canceled)

83. The method of claim 70, wherein the one or more test cells, one or more control cells, or both, are tumor cells.

84. The method of claim 70, wherein the one or more test cells are genetically modified to express the transporter at increased levels or wherein the one or more control cells are genetically modified to express the transporter at decreased levels.

85. (canceled)

86. The method of claim 70, wherein the transporter is an SLC family member.

87. (canceled)

88. The method of claim 70, wherein the transporter is MCT1.

89-97. (canceled)
